SEQUENCE LISTING (56 sequences)

SEQ ID NO: 1 - protein, 19 aa, synthetic construct
  source 1..19: note = Exemplary linker
GGGGSGGGGSGGGSGGGGS

SEQ ID NO: 2 - protein, 15 aa, synthetic construct
  source 1..15: note = Exemplary linker
GGGGSGGGGSGGGGS

SEQ ID NO: 3 - protein, 24 aa, synthetic construct
  source 1..24: note = Exemplary linker
GGGGSGGGGSGGGGSGGGSGGGGS

SEQ ID NO: 4 - protein, 29 aa, synthetic construct
  source 1..29: note = Exemplary linker
GGGGSGGGGSGGGGSGGGGSGGGSGGGGS

SEQ ID NO: 5 - protein, 5 aa, synthetic construct
  source 1..5: note = Exemplary linker
GGGGS

SEQ ID NO: 6 - protein, 10 aa, synthetic construct
  source 1..10: note = Exemplary linker
GGGGSGGGGS

SEQ ID NO: 7 - protein, 240 aa, Homo sapiens
  source 1..240: note = Wild-type human LIGHT protein (Uniprot Reference No: O43557-1)
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 8 - protein, 203 aa, synthetic construct
  source 1..203: note = Exemplary truncated LIGHT protein
VGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 9 - protein, 240 aa, synthetic construct
  source 1..240: note = Exemplary LIGHT polypeptide
MEESVVRPSVFVVDGQTDIPFTRLGRSHRRQSCSVARVGLGLLLLLMGAGLAVQGWFLLQLHWRLGEMVTRLPDGPAGSWGGGGQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 10 - protein, 122 aa, synthetic construct
  source 1..122: note = Exemplary VH sequence targeting CD371
EVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSS

SEQ ID NO: 11 - protein, 113 aa, synthetic construct
  source 1..113: note = Exemplary VL sequence targeting CD371
DIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIK

SEQ ID NO: 12 - protein, 8 aa, synthetic construct
  source 1..8: note = VH CDR1
GFTFSDYQ

SEQ ID NO: 13 - protein, 8 aa, synthetic construct
  source 1..8: note = VH CDR2
IQGGGGST

SEQ ID NO: 14 - protein, 15 aa, synthetic construct
  source 1..15: note = VH CDR3
AREMWRGDYYSGMDV

SEQ ID NO: 15 - protein, 12 aa, synthetic construct
  source 1..12: note = VL CDR1
QSVLDSYNNENN

SEQ ID NO: 16 - skipped

SEQ ID NO: 17 - protein, 9 aa, synthetic construct
  source 1..9: note = VL CDR3
QQYTSEPIT

SEQ ID NO: 18 - protein, 254 aa, synthetic construct
  source 1..254: note = Exemplary scFv (B10H4L)
EVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIK

SEQ ID NO: 19 - protein, 254 aa, synthetic construct
  source 1..254: note = Exemplary scFv (B10L4H)
DIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKGGGGSGGGGSGGGSGGGGSEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSS

SEQ ID NO: 20 - protein, 250 aa, synthetic construct
  source 1..250: note = Exemplary scFv (B10H3L)
EVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIK

SEQ ID NO: 21 - protein, 250 aa, synthetic construct
  source 1..250: note = Exemplary scFv (B10L3H)
DIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKGGGGSGGGGSGGGGSEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSS

SEQ ID NO: 22 - protein, 259 aa, synthetic construct
  source 1..259: note = Exemplary scFv (B10H5L)
EVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSGGGGSGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIK

SEQ ID NO: 23 - protein, 259 aa, synthetic construct
  source 1..259: note = Exemplary scFv (B10L5H)
DIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKGGGGSGGGGSGGGGSGGGSGGGGSEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSS

SEQ ID NO: 24 - protein, 264 aa, synthetic construct
  source 1..264: note = Exemplary scFv (B10H6L)
EVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSGGGGSGGGGSGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIK

SEQ ID NO: 25 - protein, 264 aa, synthetic construct
  source 1..264: note = Exemplary scFv (B10L6H)
DIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKGGGGSGGGGSGGGGSGGGGSGGGSGGGGSEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSS

SEQ ID NO: 26 - protein, 240 aa, synthetic construct
  source 1..240: note = Exemplary scFv (B10H1L)
EVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIK

SEQ ID NO: 27 - protein, 240 aa, synthetic construct
  source 1..240: note = Exemplary scFv (B10L1H)
DIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKGGGGSEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSS

SEQ ID NO: 28 - protein, 245 aa, synthetic construct
  source 1..245: note = Exemplary scFv (B10H2L)
EVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIK

SEQ ID NO: 29 - protein, 245 aa, synthetic construct
  source 1..245: note = Exemplary scFv (B10L2H)
DIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKGGGGSGGGGSEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSS

SEQ ID NO: 30 - protein, 120 aa, synthetic construct
  source 1..120: note = Exemplary VH sequence targeting mesothelin
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSS

SEQ ID NO: 31 - protein, 113 aa, synthetic construct
  source 1..113: note = Exemplary VL sequence targeting mesothelin
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLGQ

SEQ ID NO: 32 - protein, 20 aa, Homo sapiens
  source 1..20: note = Human IL-2 signal sequence
MYRMQLLSCIALSLALVTNS

SEQ ID NO: 33 - protein, 20 aa, Mus musculus
  source 1..20: note = Mouse IL-2 signal sequence
MYSMQLASCVTLTLVLLVNS

SEQ ID NO: 34 - protein, 20 aa, Homo sapiens
  source 1..20: note = Human kappa signal sequence
METPAQLLFLLLLWLPDTTG

SEQ ID NO: 35 - protein, 20 aa, Mus musculus
  source 1..20: note = Mouse kappa signal sequence
METDTLLLWVLLLWVPGSTG

SEQ ID NO: 36 - protein, 21 aa, Homo sapiens
  source 1..21: note = Human CD8 signal sequence
MALPVTALLLPLALLLHAARP

SEQ ID NO: 37 - protein, 18 aa, synthetic construct
  source 1..18: note = Truncated human CD8 signal sequence
MALPVTALLLPLALLLHA

SEQ ID NO: 38 - protein, 16 aa, Homo sapiens
  source 1..16: note = Human albumin signal sequence
MKWVTFISLLFSSAYS

SEQ ID NO: 39 - protein, 30 aa, Homo sapiens
  source 1..30: note = Human prolactin signal sequence
MDSKGSSQKGSRLLLLLVVSNLLLCQGVVS

SEQ ID NO: 40 - protein, 235 aa, Homo sapiens
  source 1..235: note = Human CD8 (Uniprot Reference No: P01732-1)
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 41 - protein, 247 aa, Mus musculus
  source 1..247: note = Mouse CD8 (NCBI Reference No: AAA92533.1)
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 42 - protein, 220 aa, Homo sapiens
  source 1..220: note = Human CD28 (NCBI Reference No: NP_006130)
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 43 - DNA, 81 bp, synthetic construct
  source 1..81: mol_type = other DNA; note = Exemplary nucleotide sequence encoding amino acid 153 to 179 of SEQ ID NO: 42
ttttgggtgctggtggtggttggtggagtcctggcttgctatagcttgctagtaacagtggcctttattattttctgggtg

SEQ ID NO: 44 - protein, 218 aa, Mus musculus
  source 1..218: note = Mouse CD28 (NCBI Reference No: NP 031668.3)
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 45 - protein, 164 aa, Homo sapiens
  source 1..164: note = Human CD3 zeta (NCBI Reference No: NP 932170)
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 46 - protein, 112 aa, synthetic construct
  source 1..112: note = Intracellular signaling domain comprises a CD3 zeta polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - DNA, 336 bp, synthetic construct
  source 1..336: mol_type = other DNA; note = Exemplary nucleotide sequence encoding the amino acid sequence of SEQ ID NO: 46
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 48 - DNA, 123 bp, synthetic construct
  source 1..123: mol_type = other DNA; note = Exemplary nucleic acid sequence encoding amino acids 180 to 220 of SEQ ID NO: 42
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 49 - protein, 255 aa, Homo sapiens
  source 1..255: note = 4-1BB polypeptide (NCBI Ref. No.: NP_001552)
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 50 - protein, 504 aa, synthetic construct
  source 1..504: note = CD371-targeted CAR
MALPVTALLLPLALLLHAEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKEQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 498 aa, synthetic construct
  source 1..498: note = Mesothelin-targeted CAR
MALPVTALLLPLALLLHAQVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSGGGGSGGGGSGGGGSDIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLGQEQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 179 aa, synthetic construct
  source 1..179: note = Exogenous IL-18 polypeptide
MGYRMQLLSCIALSLALVTNSGYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 53 - protein, 179 aa, synthetic construct
  source 1..179: note = Exogenous IL-33 polypeptide
MYRMQLLSCIALSLALVTNSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 54 - protein, 435 aa, Homo sapiens
  source 1..435: note = Wild-type human LTbR (Uniprot Reference No: P36941-1)
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 55 - RNA, 20 nt, synthetic construct
  source 1..20: mol_type = other RNA; note = LTbR gRNA
tggttctccgacgcatatgg

SEQ ID NO: 56 - RNA, 20 nt, synthetic construct
  source 1..20: mol_type = other RNA; note = HVEM gRNA
aaggaggacgagtacccagt

(19) United States
(12) Patent Application Publication
(10) Pub. No.: US 2026/0297517 A1
(43) Pub. Date: Oct. 1, 2026

(54) LIGHT-EXPRESSING IMMUNORESPONSIVE CELLS AND USES THEREOF

(71) Applicant: Memorial Sloan Kettering Cancer Center, New York, NY (US)

(72) Inventors: Anthony Daniyan, Somers, NY (US); Winson Cai, Elmhurst, NY (US); Renier J. Brentjens, Short Hills, NJ (US)

(73) Assignee: Memorial Sloan Kettering Cancer Center, New York, NY (US)

(21) Appl. No.: 19/144,401

(22) PCT Filed: Dec. 29, 2023
(86) PCT No.: PCT/US2023/086415

§ 371 Date: Jun. 27, 2025

Related U.S. Application Data
(60) Provisional application No. 63/436,290, filed on Dec. 30, 2022.

(51) Int. Cl. C12N 5/0783; A61K 40/11; A61K 40/31; A61K 40/42; A61P 35/00; A61P 35/02; C07K 14/705; C07K 16/28; C07K 16/30

(52) U.S. Cl. CPC C12N 5/0636; A61K 40/11; A61K 40/31; A61K 40/4224; A61K 40/4255; A61P 35/00; A61P 35/02; C07K 14/70575; C07K 16/2851; C07K 16/30; C07K 2317/565; C07K 2317/622; C07K 2319/03; C07K 2319/41; C12N 2510/00

(57) ABSTRACT

The presently disclosed subject matter provides immunoresponsive cells comprising an antigen-recognizing receptor (e.g., a chimeric antigen receptor (CAR)), and expressing LIGHT. In certain embodiments, the engineered immunoresponsive cells are antigen-directed and have enhanced immune-activating properties.

### CROSS-REFERENCE TO RELATED APPLICATIONS

This application claims the benefit of U.S. Provisional Patent Application No. 63/436,290, filed Dec. 30, 2022, the disclosure of which is incorporated by reference herein it its entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “13542-077-228_SEQ_LISTING.xml”, was created on Dec. 24, 2023, and is 67,076 bytes in size.

### 1. INTRODUCTION

The presently disclosed subject matter provides methods and compositions for enhancing the immune response toward tumors and pathogens. It relates to immunoresponsive cells comprising antigen-recognizing receptors (e.g., chimeric antigen receptors (CARs)) that are engineered to express a LIGHT (“homologous to Lymphotoxin, exhibits Inducible expression and competes with HSV Glycoprotein D for binding to Herpesvirus entry mediator, a receptor expressed on T lymphocytes”) polypeptide. These engineered immunoresponsive cells are antigen-directed, promote recruitment of other cytokines and exhibit enhanced anti-target efficacy.

### 2. BACKGROUND OF THE INVENTION

The utilization of adoptive transfer of T cells engineered to express a chimeric antigen receptor (CAR) on their cell surface is an area of growing interest in immunotherapy of cancer. With recent clinical successes on the FDA-approved autologous CD19-directed CAR T cells for the treatment of B-cell Acute Lymphoid Leukemia (ALL), there are emerging desires to translate that clinical efficacy to other liquid tumors and solid tumor malignancies (Brentjens et al., Sci Transl Med. (2013 Mar. 20); 5(177): 177ra38). Solid malignancies exhibit a new set of challenges that limit treatment efficacy compared to hematologic cancers. Some of these challenges include the lack of specific tumor-associated-antigens as well as a highly immunosuppressive tumor microenvironment that can render CAR T cells to become exhausted or dysfunctional (Sadelain et al., Cancer Discov. (2013 April); 3 (4): 388-98; Kowolik et al., Cancer Res. (2006 Nov. 15); 66 (22): 10995-1004; Davila et al., Sci Transl Med. (2014 Feb. 19); 6 (224): 224ra25). Hence, for CAR T cell therapies to be successful in solid tumors, innovative CAR designs must address some of these major roadblocks.

Unlike B-cell hematological malignancies where the CD19 target antigen is ubiquitously expressed, the heterogeneous nature of the solid tumor makes antigen-specific CAR T cells not effective when antigen-negative tumor cells are resistant to CAR T cell cytotoxicity and repopulate the tumor mass. The outgrowth of the tumor mass becomes refractory to the adoptively transferred T cells and contributes to disease relapse. Thus, new innovative treatment strategies are needed to counteract these issues to provide an effective and durable antitumor response.

### 3. SUMMARY OF THE INVENTION

The presently disclosed subject matter provides immunoresponsive cells (e.g., T cells, Natural Killer (NK) cells,) that (a) express an antigen-recognizing receptor (e.g., chimeric antigen receptors (CAR) or T cell receptor (TCR)) directed toward a target antigen of interest, and (b) express a LIGHT (“homologous to Lymphotoxin, exhibits Inducible expression and competes with HSV Glycoprotein D for binding to Herpesvirus entry mediator, a receptor expressed on T lymphocytes”) polypeptide. In certain embodiments, the immunoresponsive cell comprises a nucleotide acid encoding a LIGHT polypeptide (e.g., LIGHT polypeptide-encoding nucleic acid), in an expressible form. In certain embodiments, the LIGHT polypeptide is constitutively expressed.

In certain embodiments, the LIGHT polypeptide is secreted. In certain embodiments, the LIGHT polypeptide is not secreted (e.g., the LIGHT polypeptide is membrane bound).

In certain embodiments, the antigen-recognizing receptor is recombinantly expressed. In certain embodiments, the antigen-recognizing receptor is expressed from a vector. In certain embodiments, the exogenous LIGHT polypeptide is expressed from a vector.

In certain embodiments, the immunoresponsive cell is selected from the group consisting of a T cell, a Natural Killer (NK) cell, and a stem cell from which a lymphoid cell may be differentiated. In certain embodiments, the immunoresponsive cell is a T cell. In certain embodiments, the T cell is selected from the group consisting of a cytotoxic T lymphocyte (CTL), a regulatory T cell, and a Natural Killer T (NKT) cell. In certain embodiments, the stem cell is a pluripotent stem cell. In certain embodiments, the immunoresponsive cell is autologous. In certain embodiments, the immunoresponsive cell is allogeneic.

In certain embodiments, the antigen to which the antigen-recognizing receptor binds is a tumor antigen or a pathogen antigen. In certain embodiments, the tumor expresses LTβR or a portion thereof. In certain embodiments, the tumor cell expresses a functional LTβR. In certain embodiments, the functional LTβR comprises amino acids 249 to 435 of SEQ ID NO: 54.

In certain embodiments, the tumor is selected from the group consisting of acute myeloid leukemia (AML), pancreatic cancer, ovarian cancer, lung cancer, melanoma, colon cancer, colorectal cancer, mesothelioma, osteosarcoma, cholangiocarcinoma, and breast cancer.

In certain embodiments, the antigen is a tumor antigen. In certain embodiments, the tumor is blood cancer. In certain embodiments, the blood cancer is selected from the group consisting of acute myeloid leukemia (AML), chronic lymphocytic leukemia, B cell leukemia, multiple myeloma, lymphoblastic leukemia (ALL), Hodgkin's lymphoma, and non-Hodgkin's lymphoma. In certain embodiments, the tumor is a solid tumor. In certain embodiments, the solid tumor is selected from the group consisting of pancreatic cancer, ovarian cancer, lung cancer, melanoma, colon cancer, colorectal cancer, mesothelioma, osteosarcoma, cholangiocarcinoma, and breast cancer.

In In certain embodiments, the tumor antigen is selected from the group consisting of CD371, mesothelin, CD19, MUC16, MUC1, CAIX, CEA, CD8, CD7, CD10, CD20, CD22, CD30, CLL1, CD33, CD34, CD38, CD41, CD44, CD49f, CD56, CD74, CD123, CD133, CD138, EGP-2, EGP-40, EpCAM, Erb-B2, Erb-B3, Erb-B4, FBP, Fetal acetylcholine receptor, folate receptor-α, GD2, GD3, HER-2, hTERT, IL-13R-α2, κ-light chain, KDR, LeY, L1 cell adhesion molecule, MAGE-A1, MAGEA3, p53, MARTI, GP100, Proteinase3 (PR1), Tyrosinase, Survivin, EphA2, NKG2D ligands, NY-ESO-1, oncofetal antigen (h5T4), PSCA, PSMA, ROR1, TAG-72, VEGFR2, WT-1, BCMA, NKCS1, EGF1R, and EGFR-VIII. In certain embodiments, the tumor antigen is CD371. In certain embodiments, the tumor antigen is mesothelin.

In certain embodiments, the antigen-recognizing receptor is a T cell receptor (TCR) or a chimeric antigen receptor (CAR). In certain embodiments, the antigen-recognizing receptor is a chimeric antigen receptor (CAR). In certain embodiments, the CAR comprises an extracellular antigen-binding domain, a transmembrane domain, and an intracellular domain. In certain embodiments, the extracellular antigen-binding domain comprises an antigen-binding fragment that binds to the antigen. In certain embodiments, the antigen-binding fragment is a fragment antigen-binding (Fab), (Fab)₂, variable fragment (Fv), or a single chain variable fragment (scFv). In certain embodiments, the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, or a combination thereof. In certain embodiments, the transmembrane domain comprises a CD28 polypeptide. In certain embodiments, the intracellular domain comprises a CD3 polypeptide. In certain embodiments, the intracellular domain further comprises at least one co-stimulatory signaling region. In certain embodiments, the at least one co-stimulatory signaling region comprises a CD28 polypeptide, a 4-1BB polypeptide, an OX40 polypeptide, an ICOS polypeptide, a DAP-10 polypeptide, or a combination thereof. In certain embodiments, the at least one co-stimulatory signaling region comprises a CD28 polypeptide.

In certain embodiments, the LIGHT polypeptide comprises an amino acid sequence that is at least about 80%, at least about 85%, at least about 90%, at least about 95%, at least about 99%, or at least about 100% identical to the amino acid sequence set forth in SEQ ID NO: 7. In certain embodiments, the LIGHT polypeptide comprises the amino acid sequence set forth in SEQ ID NO: 7.

In certain embodiments, the exogenous LIGHT polypeptide enhances an immune response of the immunoresponsive cell. In certain embodiments, the exogenous LIGHT polypeptide increases anti-tumor cytokine production of the immunoresponsive cell. In certain embodiments, the anti-tumor cytokine is selected from the group consisting of IL-33, IL-5, IL-9, IL-13, IL-2, granulocyte macrophage colony-stimulating factor (GM-CSP), IFN-γ, TNF-super family, Fas ligand (Fas-L), TNF-α, perforin, granzyme B, and granzyme A.

Furthermore, the presently disclosed subject matter provides compositions comprising the immunoresponsive cells disclosed herein. In certain embodiments, the composition is a pharmaceutical composition comprising a pharmaceutically acceptable excipient.

The presently disclosed subject matter further provides methods of inhibiting growth of a cell that expresses lymphotoxin-β receptor (LTβR) or a portion thereof. In certain embodiments, the tumor cell expresses a functional LTβR. In certain embodiments, the functional LTβR comprises amino acids 249 to 435 of SEQ ID NO: 54. In certain embodiments, the method comprises contacting a cell that expresses LTβR or a portion thereof with the immunoresponsive cell or composition disclosed herein. In certain embodiments, the cell is a tumor cell.

The presently disclosed subject matter further provides methods of reducing tumor burden in a subject. In certain embodiments, the method comprises administering to a subject suffering from a tumor the immunoresponsive cell disclosed herein or the composition disclosed herein. In certain embodiments, the method reduces the number of tumor cells, reduces tumor size, and/or eradicates the tumor in the subject.

The presently disclosed subject matter also provides methods of treating a tumor in a subject. In certain embodiments, the method comprises administering to a subject suffering from a tumor the immunoresponsive cell disclosed herein or the composition disclosed herein.

The presently disclosed subject matter also provides methods of lengthening survival of a subject having a tumor. In certain embodiments, the method comprises administering to a subject suffering from a tumor the immunoresponsive cell disclosed herein or the composition disclosed herein.

In certain embodiments, the tumor expresses LTβR or a portion thereof. In certain embodiments, the tumor cell expresses a functional LTβR. In certain embodiments, the functional LTβR comprises amino acids 249 to 435 of SEQ ID NO: 54. In certain embodiments, the tumor is selected from the group consisting of acute myeloid leukemia (AML), pancreatic cancer, ovarian cancer, lung cancer, melanoma, colon cancer, colorectal cancer, mesothelioma, osteosarcoma, cholangiocarcinoma, and breast cancer. In certain embodiments, the tumor is a cancer. In certain embodiments, the tumor is a blood cancer. In certain embodiments, the blood cancer is selected from the group consisting of acute myeloid leukemia (AML), chronic lymphocytic leukemia, B cell leukemia, multiple myeloma, lymphoblastic leukemia (ALL), Hodgkin's lymphoma, and non-Hodgkin's lymphoma. In certain embodiments, the tumor is a solid tumor. In certain embodiments, the solid tumor is selected from the group consisting of pancreatic cancer, ovarian cancer, lung cancer, melanoma, colon cancer, colorectal cancer, mesothelioma, osteosarcoma, cholangiocarcinoma, and breast cancer.

In certain embodiments, the tumor expresses low detectable level of the antigen to which the antigen recognizing receptor binds. In certain embodiments, the low detectable level is less than about 1500 molecules per cell. In certain embodiments, the low detectable level is less than about 1000 molecules per cell.

The presently disclosed subject matter further provides methods of increasing immune-activating cytokine production in response to an antigen in a subject. In certain embodiments, the method comprises administering to a subject in need thereof the immunoresponsive cell disclosed herein or the composition disclosed herein. In certain embodiments, the immune-activating cytokine is selected from the group consisting of IL-2, GM-SCF, IFN-γ, TNF-super family, OX40, Fas ligand (FasL), TNF-α, perforin, granzyme B, and granzyme A.

The presently disclosed subject matter further provides methods of treating a pathogen infection or an infectious disease in a subject. In certain embodiments, the method comprises administering to a subject suffering from a pathogen infection or an infectious disease the immunoresponsive cell disclosed herein or the composition disclosed herein.

In certain embodiments, the subject is a human subject.

The presently disclosed subject matter provides nucleic acid compositions. In certain embodiments, the nucleic acid composition comprises (a) a first nucleic acid encoding an antigen-recognizing receptor and (b) a second nucleic acid encoding an exogenous LIGHT polypeptide or a fragment thereof.

In certain embodiments, the first nucleic acid is operably linked to a promoter element. In certain embodiments, the second nucleic acid is operably linked to a promoter element. In certain embodiments, the promoter is a constitutive promoter or an inducible promoter. In certain embodiments, the constitutive promoter is selected from the group consisting of a CMV promoter, an EF1α promoter, a SV40 promoter, a PGK1 promoter, a Ubc promoter, a β-actin promoter, a CAG promoter, and a retroviral LTR. In certain embodiments, the inducible promoter is selected from the group consisting of CMV promoter, an EF1α promoter, a SV40 promoter, a PGK1 promoter, a Ubc promoter, a β-actin promoter, a CAG promoter, a NFAT transcriptional response element (TRE) promoter, a CD69 promoter, a CD25 promoter, an IL-2 promoter, and an estrogen response element (ERE) promoter.

The presently disclosed subject matter also provides vectors comprising the nucleic acid compositions disclosed herein. In certain embodiments, the vector is a retroviral vector.

The presently disclosed subject matter provides cells comprising the nucleic acid compositions disclosed herein. The presently disclosed subject matter also provides cells comprising the vectors disclosed herein.

The presently disclosed subject matter further provides methods for producing an antigen-specific immunoresponsive cell. In certain embodiments, the method comprises introducing into an immunoresponsive cell the nucleic acid composition disclosed herein or the vector disclosed herein.

In certain embodiments, the method comprises introducing into an immunoresponsive cell comprising antigen-recognizing receptor a nucleic acid encoding an exogenous LIGHT polypeptide or a fragment thereof.

The presently disclosed subject matter provides kits comprising the immunoresponsive cells disclosed herein, the composition disclosed herein, the nucleic acid composition disclosed herein, or the vector disclosed herein. In certain embodiments, the kit further comprises written instructions for treating a tumor, a pathogen infection, and/or an infectious disease.

In certain embodiments, the immunoresponsive cell disclosed herein is for use in a therapy. In certain embodiments, the immunoresponsive cell disclosed herein is for use in inhibiting growth a cell expressing LTβR or a portion thereof, reducing tumor burden in a subject, treating a tumor in a subject, lengthening survival of a subject having a tumor, and/or increasing immune-activating cytokine production in response to an antigen in a subject.

In certain embodiments, the composition disclosed herein is for use in a therapy. In certain embodiments, the composition disclosed herein is for use in inhibiting growth of a cell expressing LTβR or a portion thereof, reducing tumor burden in a subject, treating a tumor in a subject, lengthening survival of a subject having a tumor, and/or increasing immune-activating cytokine production in response to an antigen in a subject.

### 4. BRIEF DESCRIPTION OF THE FIGURES

The following Detailed Description, given by way of example, but not intended to limit the presently disclosed subject matter to specific embodiments described, may be understood in conjunction with the accompanying drawings.

FIG. 1 depicts interactions between LTβR, HVEM, and TNF receptors with the LT/LIGHT family and the Ig-super family members CD160 and BTLA. LIGHT is shown in its membrane bound form, but it is also readily secreted like homotrimeric LTα.

FIG. 2 depicts LIGHT-modified CAR T cell interactions with cells expressing LTβR, HVEM, and target antigen.

FIG. 3A and FIG. 3B depict structures of antigen-recognizing receptors in accordance with the presently disclosed subject matter. DEL construct served as a negative control without the intracellular signaling domain when the antigen-recognition scFv bound to the target antigen.

FIG. 4 depicts detection of CD371-targeted CAR and LIGHT expressed on the surface of transduced human T cells. Antibodies against myc-tag and LIGHT detected surface expression of CAR and LIGHT in CD371_28z (B10H4L28z) constructs. Controls included untransduced T cell, and CD371-targeted DEL CAR T cell which lacked the intracellular signaling domain.

FIG. 5 depicts detection of mesothelin-targeted CAR and LIGHT expressed on the surface of transduced human T cells. Antibodies against myc-tag and LIGHT detected surface expression of CAR and LIGHT in Meso_28z constructs. Controls included untransduced T cell, and 1928z that served as a negative control as an irrelevant human CAR T cell construct.

FIGS. 6A to 6G depicts in vitro cytotoxicity assessment of CAR T cells using a 24-hour bioluminescent assay. The following cancer cell lines were used: U937 (CD371 expressing AML cell line), U937 CD371 KO (CD371 knocked-out AML cell line), OCI-AML3 (CD371-low/no expressing AML cell line), SET2 (CD371-low/no expressing AML cell line), and three solid tumor cell lines that did not express CD371, i.e., A375 (melanoma cell line), HT29 (colorectal adenocarcinoma cell line), and 8988t (pancreatic adenocarcinoma cell line). These cell lines transduced with GFP-firefly luciferase were used for tumor tracking. Plots were representative of three independent experiments. Data errors were analyzed with mean±SEM.

FIGS. 7A to 7D depict generation of U937 CD371 KO AML cancer cell lines with LTβR knockout, HVEM knockout, or HVEM+LTβR double knockout via the CRISPR-Cas9 system. Cells expressing GFP, and firefly luciferase were sorted for no HVEM/LTBR expression and validated by flow cytometry relative to isotype control. Flow plots of HVEM expression in HVEM KO in U937 CD371KO AML cell lines are shown in the top panel. Flow plots of LTβR expression in LTβR KO in U937 CD371KO AML cell lines are shown in the bottom panel.

FIGS. 8A to 8D depict in vitro LIGHT-mediated cytotoxicity assessment of CAR T cells using a 24-hour bioluminescent assay. Healthy human donor-derived CD371-targeted CAR T cells were cocultured with knockout tumor cells expressing GFP and firefly luciferase at different effector: tumor ratios. 24 hours later, bioluminescence was measured and plotted as a percentage of the signal detected in a coculture of non-functional CD371-CAR T cells. Plots were representative of three independent experiments. Data errors were analyzed with mean±SEM.

FIG. 9 depicts an experimental layout of the in vivo xenograft model with NB4 AML cell line.

FIGS. 10A and 10B depict assessment of in vivo anti-tumor efficacy of CD371-targeted CAR T cells with LIGHT in a xenograft flank model with the NB4 AML cell line. 5×10⁵ NB4 AML cells expressing GFP-firefly luciferase were inoculated intravenously 1 day before CAR T cell treatment. Mice were treated with 5×10⁵ CAR T cells intravenously 1 day after tumor inoculation. Mice were euthanized when tumor growth led to a 20% reduction in body weight or when mice suffered from hind limb paralysis and other signs of severe graft versus host disease (GvHD). CD371_DEL construct served as a negative control without the intracellular signaling domain when the antigen-recognition scFv bound to the target antigen. NCLT represented a non-cleave version of LIGHT.

FIGS. 11A to 11M depict flow cytometric analysis of the expression levels of mesothelin and LTβR on the surface of various solid tumor cell lines and killing capability of LIGHT-modified mesothelin-targeted CAR T cells against antigen expressing cell lines. The following PDAC cell lines were tested: AsPC1 pancreatic adenocarcinoma cell line, BxPC3 pancreatic adenocarcinoma cell line, CAPAN2 pancreatic adenocarcinoma cell line, MIAPACA2 pancreatic adenocarcinoma cell line, PAN01 pancreatic adenocarcinoma cell line, and PDAC2 Patient-Derived-Xenograft (PDX) PDAC cell lines. The following solid tumor cell lines were also tested: JMN2 mesothelioma cell line, MDA-MB-231 triple negative breast cancer cell line, SW620 colorectal cancer cell line, OS69 brain metastasis from osteosarcoma PDX cell line, and TFKIICC cholangiosarcoma PDX cell line. Healthy human donor-derived mesothelin-targeted CAR T cells were cocultured with tumor cells expressing GFP and firefly luciferase at different effector: tumor ratios. 72 hours later, bioluminescence was measured and plotted as a percentage of the signal detected in a coculture of untransduced T cells. Associated expression of mesothelin and LTβR with the corresponding cell lines were shown. Plots were representative of three independent experiments. Data errors were analyzed with mean±SEM. DEL construct served as a negative control without the intracellular signaling domain when the antigen-recognition scFv bound to the target antigen. NCLT represented a non-cleave version of LIGHT. 1928z served as a negative control as an irrelevant human CAR T cell construct. PAN01 PDAC cell line that did not express mesothelin and LTβR was used as a negative control. FIG. 11M depicts flow cytometric analysis of the expression levels of mesothelin on the surface of various PDAC cell lines. The number of molecules of mesothelin for each cell line was semi-quantitatively determined by the QIFIkit. Associated tumor lysis of both meso_28z CAR T cell and meso_28z_LIGHT CAR T cell were listed. Data was representative of four independent experiments.

FIG. 12A and FIG. 12B depict in vitro LIGHT-mediated cytotoxicity assessment of CAR T cells using a 24-hour bioluminescent assay. Healthy human donor-derived mesothelin-targeted CAR T cells were cocultured with tumor cells expressing GFP and firefly luciferase at different effector: tumor ratios. 24 hours later, bioluminescence was measured and plotted as a percentage of the signal detected in a coculture of non-functional mesothelin-CAR T cells. Plots were representative of three independent experiments. Data errors were analyzed with mean±SEM. In FIG. 12A, expression of LTβR in wildtype PDAC cell line was shown (right panel, the first row), compared to expression of LTβR in isotype control (right panel, the second row). LTβR expression in LTβR KO cell line was shown (right panel, the third row), compared to expression of LTβR in isotype control (right panel, the last row). In FIG. 12B, expression of LTβR in wildtype PDAC cell line was shown (right panel, the middle peak), LTβR expression in LTβR KO cell line was shown (right panel, the left peak), and overexpression of truncate LTβR that was without intracellular signaling domain was shown (right panel, the right peak).

FIG. 13 depicts proliferation of mesothelin-targeted CAR T cell in a 5-day coculture assay with mesothelin-positive PDAC cell lines. CAR T cells were cocultured with tumor cells at a 1:1 effector: target ratio for 5 days. The fold expansion was relative to the initial amount of CAR T cells added after 5 days of co-culture.

FIG. 14 depicts proinflammatory cytokines secretion of LIGHT-modified CAR T cells and the CAR T cells without LIGHT. 5×10⁴ CAR T cells and 5×10⁴ tumor cells were co-cultured in a 96-well round-bottom plate in 200 μL of T cell media. After 24 hours, the supernatant was collected and analyzed using the FLEXMAP 3D system (Luminex). Data representative of four separate donors and data errors were analyzed with mean±SEM.

FIGS. 15A to 15C depict assessment of in vivo anti-tumor efficacy of mesothelin-targeted CAR T cells with LIGHT in a xenograft flank model with the AsPC1 PDAC cell line. 1928z served as a negative control as an irrelevant human CAR T cell construct. NCG mice were subcutaneously injected with 2×10⁶ AsPC1 cells. Fourteen days later, mice were randomly assigned to four groups and were infused intravenously with 1×10⁶ CAR T cells. Tumor burden was measured by caliper at indicated days since CAR T cell infusion. Tumor burden (mm³) was quantified by (L×W×W)/2 in mice treated with various CAR T cell constructs at indicated days since CAR T cell infusion. Mouse survival days post CAR T cell treatment were shown by Kaplan-Meyer plot.

FIGS. 16A to 16E depict assessment of in vivo anti-tumor efficacy of mesothelin-targeted CAR T cells with LIGHT in a xenograft flank model with the MIAPACA2 PDAC cell line. 1928z served as a negative control as an irrelevant human CAR T cell construct. NCG mice were subcutaneously injected with 2×10⁶ MIAPACA2 cells. Fourteen days later, mice were randomly assigned to four groups and were infused intravenously with 2×10⁶ CAR T cells. Tumor burden was measured by bioluminescence (BLI, bioluminescence imaging) at indicated days since CAR-T cell infusion. Tumor burden (total flux) was quantified by photons/s in mice treated with various CAR-T cell constructs at indicated days since CAR-T cell infusion. Mouse survival days post CAR T cell treatment were shown by Kaplan-Meyer plot. Bioluminescence images of MIAPACA2 tumor cells in vivo after CAR T cell treatment were collected. Toxicity was assessed for the CAR T cell treatment by monitoring relative body weight changes compared to the Day 0 treatment.

FIG. 17 depicts transgene expression of the CAR constructs after retroviral transduction of primary human T cells.

FIG. 18 depicts transgene expression of the CAR constructs after retroviral transduction of human T cells by flow cytometry. CD19 CAR T cells (“1928z”) were used as a control.

FIG. 19 depicts transgene expression of the CAR construct after retroviral transduction of primary T cells.

FIGS. 20A to 20C depict in vitro cytotoxicity assay of CAR T cells with various cell lines. FIG. 20A depicts in vitro cytotoxicity assay of CAR T cells with CAPAN2 using luciferase killing assay. FIG. 20B depicts in vitro cytotoxicity assay of CAR T cells with MIAPACA2 using luciferase killing assay. FIG. 20C depicts in vitro cytotoxicity assay of CAR T cells with Panc1 using luciferase killing assay. Plots represent 3 independent experiments with 3 different human donors. Data errors were analyzed with mean±standard error of the mean (SEM).

FIG. 21 depicts flow cytometric analysis of the expression level of mesothelin on the surface of MDA-MB-231 breast cancer cell line and killing capability of LIGHT-modified mesothelin-targeted CAR T cells against MDA-MB-231 cell line. Healthy human donor-derived mesothelin-targeted CAR T cells were cocultured with tumor cells expressing GFP and firefly luciferase at different effector to tumor ratios. Bioluminescence was measured 72 hours later and plotted as a percentage of the signal detected with tumor cells alone (max bioluminescence signal). Associated expression of mesothelin with the mesothelin-positive cell line, MDA-MB-231 was shown. Plots represent 3 independent experiments. Data errors were analyzed with mean±SEM.

FIG. 22 depicts quantitative determination of cell surface antigen (mesothelin) of various PDAC cell lines and the corresponding tumor lysis % of both second-generation CAR T cell (Meso-28z) and LIGHT-CAR T cell (Meso-28z-LIGHT) at 2:1 effector to tumor ratio.

FIGS. 23A to 23F depict effects of LIGHT in its soluble form (sLIGHT) on CAR T cells and cytotoxicity of LIGHT-CAR T cells in LTβR knockout cell line. FIG. 23A depicts ELISA quantification of soluble LIGHT in cell culture media after 24 hours of incubation. FIG. 23B depicts in vitro cytolysis assessed against AsPC1. Supernatant from LIGHT-CAR T cells and second-generation CAR T cells were added to corresponding CAR T cells and in vitro cytolysis was assessed against AsPC1. FIG. 23C depicts in vitro cytolysis assessed against MIAPACA2. Supernatant from LIGHT-CAR T cells and second-generation CAR T cells were added to corresponding CAR T cells and in vitro cytolysis was assessed against MIAPACA2. FIG. 23D depicts flow cytometric analysis of LTβR expression of MIAPACA2 cells after CRISPR knockout (KO). MIAPACA2 LTβR KO cells (the second row) were also transduced with non-signaling LTβR without intracellular signaling portion (tLTBR) (the first row). MIAPACA2 wildtype (WT) cells (the third row) and MIAPACA2 isotype (the last row) were used as control. FIG. 23E depicts that MIAPACA2 LTβR KO healthy human donor-derived mesothelin-targeted CAR T cells were cocultured with tumor cells expressing GFP and firefly luciferase at different effector to tumor ratios. Bioluminescence was measured 72 hours later and plotted as a percentage of the signal detected in a coculture of non-functional Meso-DEL-CAR T cells. Plots represent 3 independent experiments. Data errors were analyzed with mean±SEM. FIG. 23F depicts that no killing advantage was observed with LIGHT CAR T cells co-cultured with the MIAPACA2 non-signaling, (truncated) tLTβR cancer cell line.

FIG. 24 depicts cytotoxicity assay with various mesothelin-directed CAR constructs and the addition of recombinant LIGHT to MIAPACA2.

FIG. 25 depicts cytotoxicity of MIAPACA2 and MIAPACA2 LTβR KO cell lines with non-functional CAR T cells (meso-DEL and meso-DEL-LIGHT) that had been activated by CD3/CD28 Dynabeads.

FIGS. 26A to 26C depict LIGHT-CAR T cell proliferation upon antigen stimulation. FIG. 26A depicts that mesothelin-directed LIGHT-CAR T cells exhibited better proliferation in a repetitive antigen stimulation assay with PDAC cell line, MIAPACA2. CAR T cells were cocultured with tumor cells at a 4:1 effector: tumor ratio for 5 days, and then CAR T cells were taken out and put onto new tumor cells at the original E:T ratio. The total fold expansion was quantified from multiplying each round of fold expansion every 5 days. Data is representative of 3 independent experiments of 3 different human donors and data errors were analyzed with mean±SEM. FIG. 26B depicts flow cytometric analysis of activation marker, IL2RA (CD25), on various CAR T cell constructs after 15 days of coculture with MIAPACA2 PDAC cell line. FIG. 26C depicts flow cytometric analysis of co-inhibitory receptors (PD-1, TIM-3, LAG-3) of various CAR T cell constructs after 15 days of coculturing with MIAPACA2 PDAC cell line.

FIGS. 27A to 27G depict LIGHT-CAR T cell secretion of proinflammatory cytokines upon antigen stimulation. CAR T cells (1928z, Meso-28z, and Meso-28z-LIGHT) were co-cultured with 1:1 (25,000) with PDAC cancer cell lines (CAPAN2, AsPC1, MIAPACA2, and PANC1) in 200 μL of media for 24 hours in 96-well plates. Cells were pellet down and the supernatant was collected for multiplex cytokine profiling using the LUMINEX FLEXMAP 3D system. Data are representative of 4 separate human donors. Data errors were analyzed with mean±SEM.

FIG. 28 depicts violin plots showing quality metrics of single cells included in downstream analysis. nCount RNA: number of RNA unique molecular identifiers (UMI); nFeature RNA: number of detected genes; nCount_ADT: number of antibody UMI; nFeature_ADT: number of detected antibodies; percent.mt: percentage of mitochondrial gene expression; HTO_margin: difference between signals for the hashtag with the highest signal and the hashtag with the second highest signal.

FIGS. 29A to 29H depict single-cell multiomics profiling of LIGHT-CAR T cells. FIG. 29A depicts volcano plot depicting differentially expressed genes between LIGHT-CAR T cells (Meso-28z-LT) and control CAR T cells (Meso-28z) at rest (t0). The x-axis indicates log fold-change of the average expression between the groups. The y-axis indicates negative log of the adjusted P-value based on Bonferroni correction using all features in the dataset. FIG. 29B depicts gene ontology (GO) terms enriched in differentially expressed genes in LIGHT-CAR T cells (Meso-28z-LT) compared to control CAR T cells (Meso-28z) at (t0). LIGHT-CAR T cells were enriched for expression of genes involved in T cell activation, signaling receptor binding, and cell secretion/export. FIG. 29C depicts volcano plot depicting differentially expressed genes between LIGHT-CAR T cells (Meso-28z-LT) and control CAR T cells (Meso-28z) 48 hours after co-culture with cancer cells (t48). FIG. 29D depicts GO terms enriched in differentially expressed genes in LIGHT-CAR T cells (Meso-28z-LT) compared to control CAR T cells (Meso-28z) 48 hours after co-culture with cancer cells (t48). LIGHT-CAR T cells were enriched for expression of genes involved in receptor-ligand activity, cytokine activity, and T cells migration/chemotaxis. FIG. 29E depicts heatmap displaying differentially expressed surface protein in LIGHT-CAR T cells (Meso-28z-LT) compared to control CAR T cells (Meso-28z) at rest (t0) and 48 hours after co-culture with cancer cells (t48). LIGHT-CAR T cells show higher expression of activation markers at both timepoints. FIG. 29F depicts weighted-nearest neighbor (WNN) UMAP of single cells (n=28,855) integrated and clustered based on a weighted combination of RNA and antibody-derived tag (ADT). Clusters or shared cell states were annotated based on conserved gene and surface protein expression across conditions and previously known markers of T cell type, proliferation, activation, cytotoxicity, and cytokines.

FIG. 29G depicts expression of a custom cytotoxicity gene set (GZMA, GZMH, GZMM, GZMK, NKG7, GNLY, PRF1) in LIGHT-CAR T cells (Meso-28z-LT) compared to control CAR T cells (Meso-28z) at rest (t0) and 48 hours after co-culture with cancer cells (t48) projected onto the WNN UMAP. FIG. 29H depicts violin plots comparing expression of indicated genes in distinct clusters of LIGHT-CAR T cells (Meso-28z-LT) compared to control CAR T cells (Meso-28z) 48 hours of co-culture with cancer cells (t48).

FIG. 30 depicts weighted-nearest neighbor (WNN) UMAP of single cells colored by condition (CAR T cell construct and timepoint) (top) and CD4⁺ vs CD8⁺ cell type (bottom).

FIG. 31 depicts expression of T cell markers (CD3, CD4, and CD8) at the RNA (top) and antibody-derived tag (ADT) (bottom) level projected on WNN UMAPs.

FIG. 32 depicts that expression of the RNA and ADT markers of T cell type, proliferation, activation, cytotoxicity, and cytokines supported CITE-seq cluster annotation. The top panel shows control CAR T cell and the bottom panel shows LIGHT-CAR T cell.

FIGS. 33A to 33C depict assessment of in vivo anti-tumor efficacy of mesothelin-targeted CAR T cells with LIGHT in a xenograft flank model with the AsPC1 PDAC cell line. FIG. 33A depicts tumor burden (mm³) quantified by caliper measurement ([L*W*W]/2) post-CAR T cell treatment (days). FIG. 33B depicts representative bioluminescent images (BLI) showing tumor growth of PDAC in untreated and CAR T cell-treated groups (various constructs) at days (D) post-CAR T cell treatment. FIG. 33C depicts Kaplan-Meyer plot showing mouse survival days post CAR T cell treatment (AsPC1).

FIGS. 34A to 34C depict assessment of in vivo anti-tumor efficacy of mesothelin-targeted CAR T cells with LIGHT in a xenograft flank model with the MIAPACA2 PDAC cell line. FIG. 34A depicts total flux (photons/second [p/s]) showing tumor burden in mice treated with various CAR T cell constructs at days post-CAR T cell treatment. Whole-body BLI via IVIS with standard error of the mean (SEM). FIG. 34B depicts representative BLI images showing tumor growth of MIAPACA2 in CAR T cell treated groups (various constructs) at days (D) post treatment. FIG. 34C depicts Kaplan-Meyer plot showing mouse survival days post-CAR T cell treatment (MIAPACA2).

FIGS. 35A to 35C depict assessment of mesothelin expression on a variety of PDX models using immunohistochemistry. FIG. 35A depicts immunohistochemistry staining of mesothelin-negative cancer cell line, Panc1, as a negative control. FIG. 35B depicts immunohistochemistry staining of mesothelin-positive cancer cell line, MDA-MB-231, as a positive control. FIG. 35C depicts immunohistochemistry staining of various PDX slides samples to validate their mesothelin expression.

FIGS. 36A to 36D depict anti-tumor response of LIGHT-CAR T cells in patient-derived orthotopic model of human PDAC. FIG. 36A depicts PDX PDAC2-Luc with Matrigel were engrafted into immunodeficient NCG mice via intra-pancreatic injection, followed by CAR T cell treatment 7 days later. Tumor growth was assessed weekly via bioluminescence imaging (BLI) using in vivo imaging (IVIS). FIG. 36B depicts representative BLI showing tumor growth in untreated and CAR T cell-treated groups at various days post-CAR T cells treatment. FIG. 36C depicts total flux (photons/second [p/s]) showing tumor burden in mice treated with various CAR T cell constructs at various days post-CAR T cell treatment. Whole-body BLI via IVIS with mean±SEM. FIG. 36D depicts Kaplan-Meyer plot showing mouse survival days post CAR T cell treatment (PDAC2).

FIGS. 37A to 37I depict assessment of toxicity of LIGHT-CAR T cells in immunocompetent mouse models and non-cancerous cell types. FIG. 37A depicts schematics of CAR designs with or without LIGHT. Anti-mCD19 ScFv constructs for the syngeneic model were illustrated. m19mt-DEL construct served as a negative CAR control without intracellular signaling upon antigen recognition. FIG. 37B depicts transgene expression of the CAR constructs after retroviral transduction of mouse T cells. Mouse CD19 CAR T cell constructs with mouse LIGHT (mLIGHT) (the last row) and without mLIGHT (the third row) were designed with myc tag to allow for detection of CAR T cells. m19mt-DEL that lacked the intracellular signaling portion of the CAR served as a negative control (the second row). Untransduced mouse T cells were used as negative control (the first row). FIG. 37C depicts that mouse CD19-CAR T cells were cocultured with tumor cells expressing GFP and firefly luciferase at different effector to tumor cell ratios. 1928z-LIGHT CAR T cells exhibited better cytotoxicity against CD19-negative cancer cell lines. Expression of mesothelin and LTβR with the corresponding cell lines were shown. FIG. 37D depicts knockout (KO) of LTβR in the B16F10 melanoma cell line abolished the killing advantage of CD19-LIGHT CAR T cells. FIG. 37E depicts mCD19-CAR T cells were engrafted into immunocompetent C57/BL6 mice followed by peripheral blood collection with retro-orbital eye bleed at days 7, 14, and 30. At the day 30 endpoint, the mice were sacrificed for full necropsy for analysis of potential toxicity in various organs and tissues. FIG. 37F depicts flow cytometry analysis of B cells, T cells, and myeloid cells from peripheral blood of C57/BL6 mice with CAR T cell infusion. FIG. 37G depicts relative weight change (percent change in initial weight) in the days following injection of various mouse CAR T cell constructs. FIG. 37H depicts that human CAR T cells expressing LIGHT were cocultured for 72 hours with human umbilical vein endothelial cells (HUVEC) stained with CSFR dye. Flow cytometry analysis displayed 2 distinct populations and no reduction in HUVEC cell number were observed with LIGHT-CAR T cells (the second row). HUVEC alone was used as control (the last row). FIG. 37I depicts quantification of HUVEC count after 72 hours of coculture with CAR T cells with and without LIGHT. Plots represent 3 independent experiments with 3 different human donors.

FIGS. 38A to 38C depict assessment of toxicity of LIGHT-CAR T cells to various organs and tissues. FIG. 38A depicts histopathological analysis images of certain organs in the 3 CAR-treated groups following full necropsy on day 30. Representative regions displaying lesion or absence of lesion at 20× magnification were stained with H&E. Scale bar was 100 microns. FIG. 38B depicts histological examination of various organs and tissues on day 30 after CAR T cell treatment. FIG. 38C depicts serum chemistry values of mice on day 30 after CAR T cell treatment. Readings higher or lower than reference range were highlighted.

### 5. DETAILED DESCRIPTION OF THE INVENTION

The presently disclosed subject matter provides cells, including genetically modified immunoresponsive cells (e.g., T cells, NK cells) comprising a combination of an antigen-recognizing receptor (e.g., a CAR or TCR) and an exogenous LIGHT polypeptide. The presently disclosed subject matter also provides methods of using such cells for inhibiting growth of a cell expressing LTβR or a portion thereof, and/or treating tumors, pathogen infections, and infectious diseases. The presently disclosed subject matter is based, at least in part, on the discovery that LIGHT can inhibit growth of a LTβR-expressing cell, and enhances the anti-tumor effect of an immunoresponsive cell comprising an antigen-recognizing receptor (e.g., a CAR-expressing T cell). It was observed that the co-expression of a LIGHT polypeptide and an antigen-recognizing receptor (e.g., a CAR) on T cells led to increased cytokine secretion, and enhanced cytolytic activity of the T cells.

Non-limiting embodiments of the presently disclosed subject matter are described by the present specification and Examples. For purposes of clarity of disclosure and not by way of limitation, the detailed description is divided into the following subsections:
5.1.—Definition
5.2.—LIGHT
5.3.—Antigen-recognizing receptors
5.4.—Cells
5.5.—Nucleic acid compositions and vectors
5.6.—Polypeptides
5.7.—Formulations and administration
5.8.—Methods of treatment
5.9.—Kits

### 5.1 Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5^{th} Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when an endogenous TCR or an exogenous CAR binds to an antigen, a formation of an immunological synapse occurs that includes clustering of many molecules near the bound receptor (e.g. CD4 or CD8, CD3γ/δ/ε/ζ, etc.). This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40 and ICOS. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

The term “antigen-recognizing receptor” as used herein refers to a receptor that is capable of recognizing a target antigen. In certain embodiments, the antigen-recognizing receptor is capable of activating an immune or immunoresponsive cell (e.g., a T cell) upon its binding to the target antigen. Non-limiting examples of antigen-recognizing receptors include native or endogenous T cell receptors (“TCRs”), and chimeric antigen receptors (“CARs”).

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab.m F(ab′)₂, and Fab fragments that lack the Fe fragment of intact antibody, clear more rapidly from the circulation, and may have less non-specific tissue binding of an intact antibody (Wahl et al., J Nucl. Med. 24:316-325 (1983). As used herein, antibodies include whole native antibodies, bispecific antibodies; chimeric antibodies; Fab, Fab′, single chain V region fragments (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (CH) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987), or IMGT numbering system (Lefranc, The Immunologist (1999); 7:132-136; Lefranc et al., Dev. Comp. Immunol. (2003); 27:55-77). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the IMGT numbering system. In certain embodiments, the CDR regions are delineated using the IMGT numbering system accessible at http://www.imgt.org/IMGT_vquest/input.

As used herein, the term “single-chain variable fragment” or “scFv” is a fusion protein of the variable regions of the heavy (V_{H}) and light chains (V_{L}) of an immunoglobulin covalently linked to form a V_{H}::V_{L} heterodimer. The V_{H} and V_{L} are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chern. 80 (6): 1910-1917 (2008) and WO 2014/087010, the contents of which are hereby incorporated by reference in their entireties. In certain embodiments, the linker is a G4S linker.

In certain embodiments, the linker comprises the amino acid sequence set forth in SEQ ID NO: 1, which is provided below:

[SEO ID NO: 1]
GGGGSGGGGSGGGSGGGGS

In certain embodiments, the linker comprise the amino acid sequence set forth in SEQ ID NO: 2, which is provided below:

[SEQ ID NO: 1]
GGGGSGGGGSGGGGS

In certain embodiments, the linker comprises the amino acid sequence set forth in SEQ ID NO: 3, which is provided below:

[SEQ ID NO: 3]
GGGGSGGGGSGGGGSGGGSGGGGS

In certain embodiments, the linker comprises the amino acid sequence set forth in SEQ ID NO: 4, which is provided below:

[SEQ ID NO: 4]
GGGGSGGGGSGGGGSGGGGSGGGSGGGGS

In certain embodiments, the linker comprises the amino acid sequence set forth in SEQ ID NO: 5, which is provided below:

[SEQ ID NO: 5]
GGGGS

In certain embodiments, the linker comprises the amino acid sequence set forth in SEQ ID NO: 6, which is provided below:

[SEQ ID NO: 6]
GGGGSGGGGS

Despite removal of the constant regions and the introduction of a linker, scFv proteins retain the specificity of the original immunoglobulin. Single chain Fv polypeptide antibodies can be expressed from a nucleic acid including V_{H}- and V_{L}-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication No. 20050196754. Antagonistic scFvs having inhibitory activity have been described (see, e.g., Zhao et al., Hyrbidoma (Larchmt) 2008 27 (6): 455-51; Peter et al., J Cachexia Sarcopenia Muscle 2012 Aug. 12; Shieh et al., J Imunol 2009 183 (4): 2277-85; Giomarelli et al., Thromb Haemost 2007 97 (6): 955-63; Fife et al., J Clin Invst 2006 116 (8): 2252-61; Brocks et al., Immunotechnology 1997 3 (3): 173-84; Moosmayer et al., Ther Immunol 1995 2 (10:31-40). Agonistic scFvs having stimulatory activity have been described (see, e.g., Peter et al., J Bioi Chem 2003 25278 (38): 36740-7; Xie et al., Nat Biotech 1997 15 (8): 768-71; Ledbetter et al., Crit Rev Immunol 1997 17 (5-6): 427-55; Ho et al., BioChim Biophys Acta 2003 1638 (3): 257-66).

The term “chimeric antigen receptor” or “CAR” as used herein refers to a molecule comprising an extracellular antigen-binding domain that is fused to an intracellular domain that is capable of activating or stimulating an immunoresponsive cell, and a transmembrane domain. In certain embodiments, the extracellular antigen-binding domain of a CAR comprises a scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain.

By “substantially identical” or “substantially homologous” is meant a polypeptide or nucleic acid molecule exhibiting at least about 50% identical or homologous to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% identical or homologous to the sequence of the amino acid or nucleic acid used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 1988, 4:11-17) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol., 1970, 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al., J. Mol. Biol., 1990, 215:403-410. BLAST protein searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., Nucleic Acids Res., 1997, 25 (17): 3389-3402. When utilizing BLAST and Gapped BLAST programs, the default parameters of the respective programs (e.g., XBLAST and NBLAST) can be used.

An “effective amount” is an amount sufficient to affect a beneficial or desired clinical result upon treatment. An effective amount can be administered to a subject in one or more doses. In certain embodiments, an effective amount can be an amount that is sufficient to palliate, ameliorate, stabilize, reverse or slow the progression of the disease, or otherwise reduce the pathological consequences of the disease. The effective amount can be determined by a physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

By “disease” is meant any condition, disease or disorder that damages or interferes with the normal function of a cell, tissue, or organ, e.g., neoplasia, and pathogen infection of cell.

As used herein, the term “endogenous” refers toa nucleic acid molecule or polypeptide that is normally expressed in a cell or tissue.

As used herein, the term “exogenous” refers toa nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By a “heterologous nucleic acid molecule or polypeptide” is meant a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

By “modulate” is meant positively or negatively alter. Exemplary modulations include a about 1%, about 2%, about 5%, about 10%, about 25%, about 50%, about 75%, or about 100% change.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated cell” is meant a cell that is separated from the molecular and/or cellular components that naturally accompany the cell.

The term “antigen-binding domain” as used herein refers to a domain capable of specifically binding a particular antigenic determinant or set of antigenic determinants present on a cell.

By “neoplasm” is meant a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasia can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasia include cancers, such as sarcomas, carcinomas, or plasmacytomas (malignant tumor of the plasma cells). The neoplasia can be a primary tumor or primary cancer. In addition, the neoplasm can be in metastatic status.

By “receptor” is meant a polypeptide, or portion thereof, present on a cell membrane that selectively binds one or more ligand.

By “recognize” is meant selectively binds to a target. A T cell that recognizes a tumor can expresses a receptor (e.g., a TCR or CAR) that binds to a tumor antigen.

By “reference” or “control” is meant a standard of comparison. For example, the level of scFv-antigen binding by a cell expressing a CAR and an scFv may be compared to the level of scFv-antigen binding in a corresponding cell expressing CAR alone.

By “secreted” is meant a polypeptide that is released from a cell via the secretory pathway through the endoplasmic reticulum, Golgi apparatus, and as a vesicle that transiently fuses at the cell plasma membrane, releasing the proteins outside of the cell.

By “signal sequence” or “leader sequence” is meant a peptide sequence (e.g., 5, 10, 15, 20, 25 or 30 amino acids) present at the N-terminus of newly synthesized proteins that directs their entry to the secretory pathway.

By “soluble” is meant a polypeptide that is freely diffusible in an aqueous environment (e.g., not membrane bound).

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, guinea pigs, rabbits, dogs, cats, sheep, pigs, goats, cattle, horses, and non-human primates such as apes and monkeys. In certain embodiments, the subject is a human.

Other aspects of the presently disclosed subject matter are described in the following disclosure and are within the ambit of the presently disclosed subject matter.

### 5.2 Light

The presently disclosed immunoresponsive cells express an antigen-recognizing receptor and a LIGHT polypeptide.

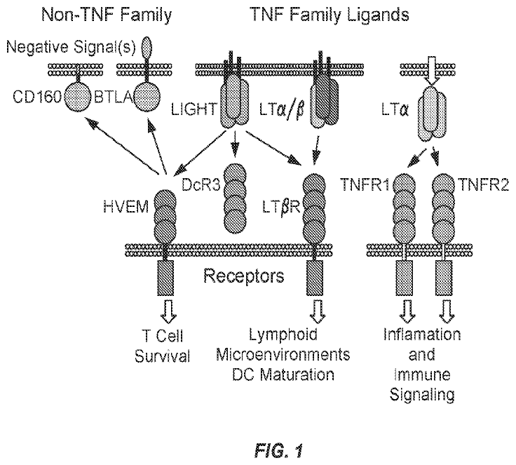

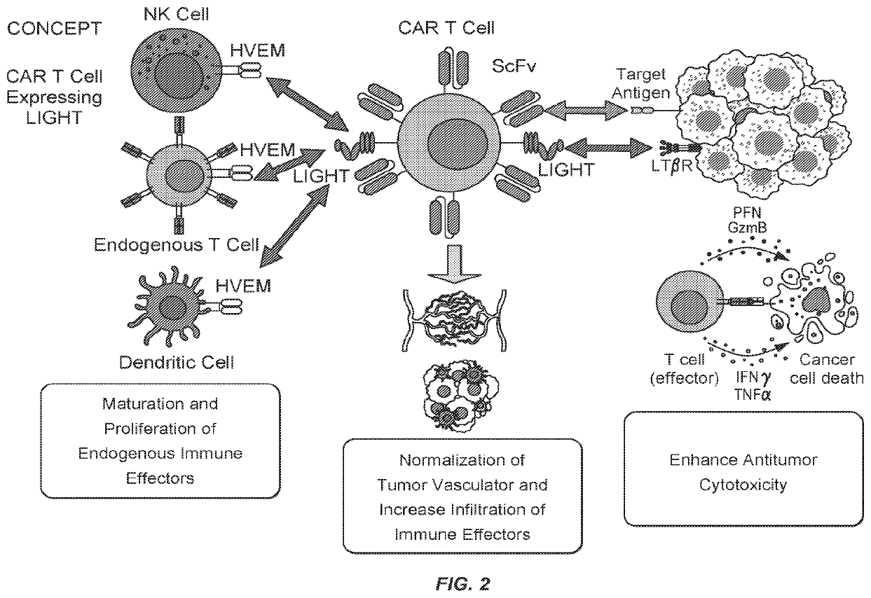

LIGHT (“homologous to lymphotoxin, exhibits inducible expression and competes with HSV glycoprotein D for binding to herpesvirus entry mediatory, a receptor expressed on T lymphocytes”), also known as tumor necrosis factor superfamily member 14 (TNFSF14 or CD258) functions as a soluble and cell surface-bound membrane protein. LIGHT has been found to primarily be expressed on activated T cells, activated Natural Killer (NK) cells, and immature dendritic cells (DCs) exhibiting inducible expression (Wang et al., J Clin Invest, 2001, 108(12): 1771-80). LIGHT functions as a homotrimer to its two primary receptors: Herpes Virus Entry Mediator (HVEM and Lymphotoxin-β Receptor (LTβR) (Mauri et al., Immunity, 1998, 8(1): 21-30). Though not well elucidated, the interaction between LIGHT and its receptors is illustrated in FIG. 1 and FIG. 2. LIGHT-HVEM interaction is reported to be associated with various antitumor responses (Holmes et al., Proc Natl Acad Sci USA, 2014, 111 (52):E5688-96; Fan et al., Blood, 2006, 107 (4): 1342-51). LIGHT signaling via these receptors seems to be cell-type dependent, but both interactions have been implicated to play a role in immune-related tumor biology.

LIGHT-HVEM interaction is reported to be responsible for the immune-stimulatory function of LIGHT. HVEM expressed on other lymphocytes, NK cells, smooth muscle cells, and epithelial cells can trigger the co-stimulatory ligand LIGHT to activate and proliferate T cells, NK cells, and maturation of DCs (9,10). LIGHT is also responsible for NK-DC crosstalk that occurs in the priming of de novo antitumor response (11). In addition, the costimulatory effect of LIGHT-HVEM interaction seems to be independent of the CD28 costimulatory interaction in the CAR T cell design (12). Thus, incorporation of LIGHT may lead to an additive or synergistic effect for T cell activation, proliferation, and survival. Also, the LIGHT-HVEM interaction can trigger T cells and NK cells to produce more IFNγ and GM-CSF, which are pro-inflammatory cytokines commonly associated with enhanced antitumor response (13).

Besides binding to HVEM, LIGHT also binds to LTβR found on the surface of a variety of epithelial, stromal, immature DC, and myeloid cells, but not on lymphocytes (Giles et al., Front Immunol, 2018, 9:2585). In addition, LIGHT has been reported to play a role in repairing chaotic or dysregulated tumor vasculature and assisting effector cells tracking and infiltration into solid tumors (He et al., J Pathol, 2018, 245 (2): 209-221; Zhang et al., Cell Res, 2004, 14(2): 117-24).

In certain embodiments, the LIGHT polypeptide is a human LIGHT polypeptide. In certain embodiments, the LIGHT polypeptide is a wild-type human LIGHT protein or a fragment thereof. In certain embodiments, the wild-type human LIGHT protein comprises the amino acid sequence with a Uniprot Reference No: O43557-1 (SEQ ID NO: 7). SEQ ID NO: 7 is provided below.

[SEQ ID NO: 7]
MEESVVRPSVFVVDGQTDIPFTRLGRSHRRQSCSVARVGLGLLLLLMGAG
LAVQGWFLLQLHWRLGEMVTRLPDGPAGSWEQLIQERRSHEVNPAAHLTG
ANSSLTGSGGPLLWETQLGLAFLRGLSYHDGALVVTKAGYYYIYSKVQLG
GVGCPLGLASTITHGLYKRTPRYPEELELLVSQQSPCGRATSSSRVWWDS
SFLGGVVHLEAGEKVVVRVLDERLVRLRDGTRSYFGAFMV

In certain embodiments, a human LIGHT polypeptide comprises an extracellular domain, a transmembrane domain, and a cytoplasmic domain. In certain embodiments, the extracellular domain comprises amino acids 59 to 240 of SEQ ID NO: 7. In certain embodiments, the transmembrane domain comprises amino acids 38 to 58 of SEQ ID NO: 7. In certain embodiments, the cytoplasmic domain comprises amino acids 1 to 37 of SEQ ID NO: 7.

In certain embodiments, the LIGHT polypeptide comprises or consists of an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 7 or a fragment thereof, and/or may comprise up to one or up to two or up to three conservative amino acid substitutions. In certain embodiments, the LIGHT polypeptide comprises or consists of an amino acid sequence that is a consecutive portion of SEQ ID NO: 7, which is at least about 5, at least about 10, at least about 15, at least about 20, at least about 25, at least about 30, at least about 40, or at least about 50, and up to 240 amino acids in length. In certain embodiments, the LIGHT peptide comprises or consists of an amino acid sequence of amino acids 1 to 240, 1 to 58, 1 to 60, 1 to 30, 1 to 35, 1 to 37, 1 to 50, 35 to 50, 38 to 50, 38 to 58, 51 to 100, 59 to 100, 59 to 240, 74 to 240, 100 to 150, 150 to 200, or 200 to 240 of SEQ ID NO: 7.

In certain embodiments, the LIGHT polypeptide comprises or consists of an amino acid sequence positioned within the cytoplasmic domain of LIGHT. For example, in certain embodiments, the LIGHT polypeptide comprises or consists of an amino acid sequence positioned within amino acids 1 to 37 of SEQ ID NO: 7.

In certain embodiments, the LIGHT polypeptide comprises or consists of an amino acid sequence positioned within the transmembrane domain of LIGHT. For example, in certain embodiments, the LIGHT polypeptide comprises or consists of an amino acid sequence positioned within amino acids 38 to 58 of SEQ ID NO: 7.

In certain embodiments, the LIGHT polypeptide comprises or consists of an amino acid sequence positioned within the extracellular domain of LIGHT. For example, in certain embodiments, the LIGHT polypeptide comprises or consists of an amino acid sequence positioned within amino acids 59 to 240 of SEQ ID NO: 7. In certain embodiments, the LIGHT polypeptide comprises or consists of an amino acid sequence positioned within amino acids 74 to 240 of SEQ ID NO: 7.

In certain embodiments, the LIGHT polypeptide comprises or consists of an amino acid sequence that is at least about 80%, at least about 85%, at least about 90%, at least about 95%, at least about 96%, at least about 97%, at least about 98%, at least about 99%, or about 100% identical to amino acids 1 to 37 of SEQ ID NO: 7. In certain embodiments, the LIGHT polypeptide comprises or consists of amino acids 1 to 37 of SEQ ID NO: 7.

In certain embodiments, the LIGHT polypeptide comprises or consists of an amino acid sequence that is at least about 80%, at least about 85%, at least about 90%, at least about 95%, at least about 96%, at least about 97%, at least about 98%, at least about 99%, or about 100% identical to amino acids 38 to 58 of SEQ ID NO: 7. In certain embodiments, the LIGHT polypeptide comprises or consists of amino acids 38 to 58 of SEQ ID NO: 7.

In certain embodiments, the LIGHT polypeptide comprises or consists of an amino acid sequence that is at least about 80%, at least about 85%, at least about 90%, at least about 95%, at least about 96%, at least about 97%, at least about 98%, at least about 99%, or about 100% identical to amino acids 59 to 240 of SEQ ID NO: 7. In certain embodiments, the LIGHT polypeptide comprises or consists of amino acids 59 to 240 of SEQ ID NO: 7. In certain embodiments, the LIGHT polypeptide comprises or consists of an amino acid sequence that is at least about 80%, at least about 85%, at least about 90%, at least about 95%, at least about 96%, at least about 97%, at least about 98%, at least about 99%, or about 100% identical to amino acids 74 to 240 of SEQ ID NO: 7. In certain embodiments, the LIGHT polypeptide comprises or consists of amino acids 74 to 240 of SEQ ID NO: 7.

In certain embodiments, the LIGHT polypeptide is cleavable. In certain embodiments, the LIGHT polypeptide is cleaved by one or more proteases. In certain embodiments, the LIGHT polypeptide is secretable.

In certain embodiments, the LIGHT polypeptide comprises or consists of the amino acid sequence set forth in SEQ ID NO: 7.

In certain embodiments, the LIGHT polypeptide is a truncated LIGHT protein. In certain embodiments, the LIGHT polypeptide comprises or consists of the amino acid sequence set forth in SEQ ID NO: 8.

[SEQ ID NO: 8]
VGLGLLLLLMGAGLAVQGWFLLQLHWRLGEMVTRLPDGPAGSWEQLIQER
RSHEVNPAAHLTGANSSLTGSGGPLLWETQLGLAFLRGLSYHDGALVVTK
AGYYYIYSKVQLGGVGCPLGLASTITHGLYKRTPRYPEELELLVSQQSPC
GRATSSSRVWWDSSFLGGVVHLEAGEKVVVRVLDERLVRLRDGTRSYFGA
FMV

In certain embodiments, the LIGHT polypeptide is not cleavable. In certain embodiments, the LIGHT polypeptide is not secretable. In certain embodiments, the LIGHT polypeptide is membrane bound. In certain embodiments, the LIGHT polypeptide is a mutated LIGHT protein or a fragment thereof. In certain embodiments, the LIGHT polypeptide is a mutated human LIGHT protein or a fragment thereof. In certain embodiments, the LIGHT polypeptide comprises one or more mutations that are capable of preventing the LIGHT polypeptide from cleavage (e.g., proteolytic cleavage). In certain embodiments, the one or more mutations are within the extracellular domain of the LIGHT, e.g., amino acids 59 to 240 of SEQ ID NO: 7.

In certain embodiments, the LIGHT polypeptide comprises or consists of an amino acid sequence that is at least about 80%, at least about 85%, at least about 90%, at least about 95%, at least about 96%, at least about 97%, at least about 98%, at least about 99%, or about 100% identical to the amino acid sequence set forth in SEQ ID NO: 9. In certain embodiments, the LIGHT polypeptide comprises or consists of the amino acid sequence set forth in SEQ ID NO: 9.

[SEQ ID NO: 9]
MEESVVRPSVFVVDGQTDIPFTRLGRSHRRQSCSVARVGLGLLLLLMGAG
LAVQGWFLLQLHWRLGEMVTRLPDGPAGSWGGGGQERRSHEVNPAAHLTG
ANSSLTGSGGPLLWETQLGLAFLRGLSYHDGALVVTKAGYYYIYSKVQLG
GVGCPLGLASTITHGLYKRTPRYPEELELLVSQQSPCGRATSSSRVWWDS
SFLGGVVHLEAGEKVVVRVLDERLVRLRDGTRSYFGAFMV

### 5.3. Antigen-Recognizing Receptors

The presently disclosed immunoresponsive cells express an antigen-recognizing receptors and a LIGHT polypeptide. In certain embodiments, the antigen-recognizing receptor binds to a target antigen.

### 5.3.1. Target Antigens

In certain embodiment, the target antigen is a tumor antigen. Any tumor antigen (antigenic peptide) can be used in the tumor-related embodiments described herein. Sources of antigen include, but are not limited to, cancer proteins. The antigen can be expressed as a peptide or as an intact protein or portion thereof. The intact protein or a portion thereof can be native or mutagenized.

In certain embodiments, the tumor is blood cancer. Non-limiting examples of blood cancer include acute myeloid leukemia (AML), chronic lymphocytic leukemia, B cell leukemia, multiple myeloma, lymphoblastic leukemia (ALL), Hodgkin's lymphoma, and non-Hodgkin's lymphoma.

In certain embodiments, the tumor is a solid tumor. Non-limiting examples of solid tumor include pancreatic cancer, ovarian cancer, lung cancer, melanoma, colon cancer, colorectal cancer, mesothelioma, osteosarcoma, cholangiocarcinoma, and breast cancer.

Non-limiting examples of tumor antigens include CD371, mesothelin, CD19, MUC16, MUC1, CAIX, CEA, CD8, CD7, CD10, CD20, CD22, CD30, CLL1, CD33, CD34, CD38, CD41, CD44, CD49f, CD56, CD74, CD123, CD133, CD138, EGP-2, EGP-40, EpCAM, Erb-B2, Erb-B3, Erb-B4, FBP, Fetal acetylcholine receptor, folate receptor-α, GD2, GD3, HER-2, hTERT, IL-13R-α2, κ-light chain, KDR, LeY, L1 cell adhesion molecule, MAGE-A1, MAGEA3, p53, MARTI, GP100, Proteinase3 (PR1), Tyrosinase, Survivin, EphA2, NKG2D ligands, NY-ESO-1, oncofetal antigen (h5T4), PSCA, PSMA, ROR1, TAG-72, VEGFR2, WT-1, BCMA, NKCS1, EGF1R, and EGFR-VIII.

In certain embodiments, the target antigen is CD371. In certain embodiments, the target antigen is human CD371. In certain embodiments, the target antigen is mesothelin. In certain embodiments, the target antigen is human mesothelin.

In certain embodiments, the target antigen is a pathogen antigen. Non-limiting examples of pathogen include a virus, bacteria, fungi, parasite and protozoa capable of causing disease.

Non-limiting examples of viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairn viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1-internally transmitted; class 2=parenterally transmitted (i.e. Hepatitis C); Norwalk and related viruses, and astroviruses).

Non-limiting examples of bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorjeri, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtheriae, corynebacterium sp., Erysipelothrix rhusiopathiae, Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

### 5.3.2. Chimeric Antigen Receptor (CAR)

In certain embodiments, the antigen-recognizing receptor is a CAR. CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARS. “First generation” CARs are typically composed of an extracellular antigen-binding domain (e.g., an scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular signaling domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ). In certain embodiments, the antigen-recognizing receptor is a first-generation CAR. In certain embodiments, the antigen-recognizing receptor is a second-generation CAR.

In certain embodiments, the CAR comprises an extracellular antigen-binding domain that binds to a target antigen, a transmembrane domain, and an intracellular domain.

### 5.3.2.1 Extracellular Antigen-Binding Domain of A CAR

In certain embodiments, the extracellular antigen-binding domain is a single chain variable fragment (scFv). In certain embodiments, the scFv is a human scFv. In certain embodiments, the scFv is a humanized scFv. In certain embodiments, the scFv is a murine scFv. In certain embodiments, the extracellular antigen-binding domain is a Fab. In certain embodiments, the Fab is crosslinked. In certain embodiments, the extracellular antigen-binding domain is a F(ab)₂.

Any of the foregoing molecules may be comprised in a fusion protein with a heterologous sequence to form the extracellular antigen-binding domain. In certain embodiments, the extracellular antigen-binding domain binds to the target antigen with a dissociation constant (K_{d}) of about 1×10⁻⁶ M or less, e.g., about 1×10⁻⁷ M or less, about 1×10⁻⁸ M or less, about 1×10⁻⁹ M or less, about 1×10⁻¹⁰ M or less, or about 1×10⁻¹¹ M or less. In certain embodiments, the extracellular antigen-binding domain binds to the target antigen with a K_{d} of about 1×10⁻⁸ M or less. In certain embodiments, the extracellular antigen-binding domain binds to the target antigen with a K_{d} of about 1×10⁻⁹ M.

Binding of the extracellular antigen-binding domain can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a γ counter or a scintillation counter or by autoradiography. In certain embodiments, the extracellular antigen-binding domain is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet). In one embodiment, the CD371-targeted human scFv is labeled with GFP.

In certain embodiments, the extracellular antigen-binding domain comprises a heavy chain variable region (V_{H}) and a light chain variable region (V_{L}).

In certain embodiments, the extracellular antigen-binding domain binds to CD371. In certain embodiments, the extracellular antigen-binding domain binds to human CD371. In certain embodiments, the V_{H} comprises an amino acid sequence that is at least about 80% (e.g., at least about 85%, at least about 90%, at least about 95%, at least about 99%, or about 100%) identical or homologous to the amino acid sequence set forth in SEQ ID NO: 8. For example, the V_{H} comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical or homologous to SEQ ID NO: 10. In certain embodiments, the V_{H} comprises the amino acid sequence set forth in SEQ ID NO: 10. SEQ ID NO: 10 is provided in Table 1 below.

In certain embodiments, the V_{L} comprises an amino acid sequence that is at least about 80% (e.g., at least about 85%, at least about 90%, at least about 95%, at least about 99%, or about 100%) identical or homologous to the amino acid sequence set forth in SEQ ID NO: 11. For example, the V_{L} comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical or homologous to SEQ ID NO: 11. In certain embodiments, the V_{L} comprises the amino acid sequence set forth in SEQ ID NO: 11. SEQ ID NO: 11 is provided in Table 1 below.

In certain embodiments, the V_{H} comprises the amino acid sequence set forth in SEQ ID NO: 10, and the V_{L} comprises the amino acid sequence set forth in SEQ ID NO: 11.

In certain embodiments, the V_{H} comprises a CDR1, a CDR2, and a CDR3 of the V_{H} sequence set forth in SEQ ID NO: 10.

In certain embodiments, the V_{H} comprises a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 1. In certain embodiments, the CDRs are identified according to the IMGT numbering system.

In certain embodiments, the V_{L} comprises a CDR1, a CDR2, and a CDR3 of the V_{L} sequence set forth in SEQ ID NO: 11.

In certain embodiments, the V_{L} comprises a CDR1 comprising the amino acid sequence set forth in SEQ ID NO: 15 or a conservative modification thereof, a CDR2 comprising the amino acid sequence set forth in SEQ ID NO: 16 or a conservative modification thereof, and a CDR3 comprising the amino acid sequence set forth in SEQ ID NO: 17 or a conservative modification thereof. SEQ ID NOs: 15-17 are provided in Table 1. In certain embodiments, the CDRs are identified according to the IMGT numbering system.

In certain embodiments, the V_{H} comprises a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof; and the V_{L} comprises a CDR1 comprising the amino acid sequence set forth in SEQ ID NO: 15 or a conservative modification thereof, a CDR2 comprising the amino acid sequence set forth in SEQ ID NO: 16 or a conservative modification, and a CDR3 comprising the amino acid sequence set forth in SEQ ID NO: 17 or a conservative modification thereof.

In certain embodiments, the V_{H} comprises a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and the V_{L} comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17.

TABLE 1

| CDRs | 1 | 2 | 3 |
|---|---|---|---|
| V_{H} | GFTFSDYQ | IQGGGGST | AREMWRGDYYSGMDV |
| | [SEQ ID NO: 12] | [SEQ ID NO: 13] | [SEQ ID NO: 14] |
| V_{L} | QSVLDSYNNENN | WAS | QQYTSEPIT |
| | [SEQ ID NO: 15] | [SEQ ID NO: 16] | [SEQ ID NO: 17] |

| | |
|---|---|
| Full V_{H} | EVQLLESGGGLVQPGGSLRLSCAASGFTFSDYQMSWVRQAPGKGLEWVSGIQGGGGST |
| | YYADSVKGRFTISRDNSKNTLYLQMNSLRAEDTAVYYCAREMWRGDYYSGMDVWGQGT |
| | TVTVSS [SEQ ID NO: 10] |
| Full V_{L} | DIVMTQSPDSLAVSLGERATINCKSSQSVLDSYNNENNLAWYQQKPGQPPKLLIYWAS |
| | TRESGVPDRFSGSGSGTDFTLTISSLQAEDVAVYYCQQYTSEPITFGQGTKVEIK |
| | [SEQ ID NO: 11] |

In certain embodiments, the extracellular antigen binding domain comprises an scFv. In certain embodiments, the V_{H} and V_{L} are linked via a linker. In certain embodiments, the V_{H} and V_{L} are linked via a linker. In certain embodiments, the linker comprises the amino acid sequence set forth in SEQ ID NO: 1. In certain embodiments, the linker comprises the amino acid sequence set forth in SEQ ID NO: 2. In certain embodiments, the linker comprises the amino acid sequence set forth in SEQ ID NO: 3. In certain embodiments, the linker comprises the amino acid sequence set forth in SEQ ID NO: 4. In certain embodiments, the linker comprises the amino acid sequence set forth in SEQ ID NO: 5. In certain embodiments, the linker comprises the amino acid sequence set forth in SEQ ID NO: 6.

In certain embodiments, the variable regions within the extracellular antigen binding domain have to be linked one after another such that at the N-terminus of the extracellular antigen-binding domain, a V_{H} is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

In certain embodiments, the scFv comprises a V_{H} comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the V_{H} and V_{L} are linked via a linker that comprises the amino acid sequence set forth in SEQ ID NO: 1. In certain embodiments, the variable regions are positioned from the N- to the C-terminus: V_{H}-V_{L}. In certain embodiments, the scFv is designated as “B10H4L”. In certain embodiments, the scFv comprises the amino acid sequence set forth in SEQ ID NO: 18. SEQ ID NO: 18 is provided below.

[SEQ ID NO: 18]
EVQLLESGGGLVQPGGSLRLSCAASGFTFSDYQMSWVRQAPGKGLEWVSG
IQGGGGSTYYADSVKGRFTISRDNSKNTLYLQMNSLRAEDTAVYYCAREM
WRGDYYSGMDVWGQGTTVTVSSGGGGSGGGGSGGGSGGGGSDIVMTQSPD
SLAVSLGERATINCKSSQSVLDSYNNENNLAWYQQKPGQPPKLLIYWAST
RESGVPDRFSGSGSGTDFTLTISSLQAEDVAVYYCOQYTSEPITFGQGTK
VEIK

In certain embodiments, the scFv comprises a V_{H} comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the V_{H} and V_{L} are linked via a linker that comprises the amino acid sequence set forth in SEQ ID NO: 1. In certain embodiments, the variable regions are positioned from the N- to the C-terminus: V_{L}-V_{H}. In certain embodiments, the scFv is designated as “B10L4H”. In certain embodiments, the scFv comprises the amino acid sequence set forth in SEQ ID NO: 19. SEQ ID NO: 19 is provided below.

[SEQ ID NO: 19]
DIVMTQSPDSLAVSLGERATINCKSSQSVLDSYNNENNLAWYQQKPGQPP
KLLIYWASTRESGVPDRFSGSGSGTDFTLTISSLQAEDVAVYYCQQYTSE
PITFGQGTKVEIKGGGGSGGGGSGGGSGGGGSEVQLLESGGGLVQPGGSL
RLSCAASGFTFSDYQMSWVRQAPGKGLEWVSGIQGGGGSTYYADSVKGRF
TISRDNSKNTLYLQMNSLRAEDTAVYYCAREMWRGDYYSGMDVWGQGTTV
TVSS

In certain embodiments, the scFv comprises a V_{H} comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the V_{H} and V_{L} are linked via a linker that comprises the amino acid sequence set forth in SEQ ID NO: 2. In certain embodiments, the variable regions are positioned from the N- to the C-terminus: V_{H}-V_{L}. In certain embodiments, the scFv is designated as “B10H3L”. In certain embodiments, the scFv comprises the amino acid sequence set forth in SEQ ID NO: 20. SEQ ID NO: 20 is provided below.

[SEQ ID NO: 20]
EVQLLESGGGLVQPGGSLRLSCAASGFTFSDYQMSWVRQAPGKGLEWVSG
IQGGGGSTYYADSVKGRFTISRDNSKNTLYLQMNSLRAEDTAVYYCAREM
WRGDYYSGMDVWGQGTTVTVSSGGGGSGGGGSGGGGSDIVMTQSPDSLAV
SLGERATINCKSSQSVLDSYNNENNLAWYQQKPGQPPKLLIYWASTRESG
VPDRFSGSGSGTDFTLTISSLQAEDVAVYYCQQYTSEPITFGQGTKVEIK

In certain embodiments, the scFv comprises a V_{H} comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the V_{H} and V_{L} are linked via a linker that comprises the amino acid sequence set forth in SEQ ID NO: 2. In certain embodiments, the variable regions are positioned from the N- to the C-terminus: V_{L}-V_{H}. In certain embodiments, the scFv is designated as “B10L3H”. In certain embodiments, the scFv comprises the amino acid sequence set forth in SEQ ID NO: 21. SEQ ID NO: 21 is provided below.

[SEQ ID NO: 21]
DIVMTQSPDSLAVSLGERATINCKSSQSVLDSYNNENNLAWYQQKPGQPP
KLLIYWASTRESGVPDRFSGSGSGTDFTLTISSLQAEDVAVYYCQQYTSE
PITFGQGTKVEIKGGGGSGGGGSGGGGSEVQLLESGGGLVQPGGSLRLSC
AASGFTFSDYQMSWVRQAPGKGLEWVSGIQGGGGSTYYADSVKGRFTISR
DNSKNTLYLQMNSLRAEDTAVYYCAREMWRGDYYSGMDVWGQGTTVTVSS

In certain embodiments, the scFv comprises a V_{H} comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the V_{H} and V_{L} are linked via a linker that comprises the amino acid sequence set forth in SEQ ID NO: 3. In certain embodiments, the variable regions are positioned from the N- to the C-terminus: V_{H}-V_{L}. In certain embodiments, the scFv is designated as “B10H5L”. In certain embodiments, the scFv comprises the amino acid sequence set forth in SEQ ID NO: 22. SEQ ID NO: 22 is provided below.

[SEQ ID NO: 22]
EVQLLESGGGLVQPGGSLRLSCAASGFTFSDYQMSWVRQAPGKGLEWVSG
IQGGGGSTYYADSVKGRFTISRDNSKNTLYLQMNSLRAEDTAVYYCAREM
WRGDYYSGMDVWGQGTTVTVSSGGGGSGGGGSGGGGSGGGSGGGGSDIVM
TQSPDSLAVSLGERATINCKSSQSVLDSYNNENNLAWYQQKPGQPPKLLI
YWASTRESGVPDRFSGSGSGTDFTLTISSLQAEDVAVYYCQQYTSEPITF
GQGTKVEIK

In certain embodiments, the scFv comprises a V_{H} comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the V_{H} and V_{L} are linked via a linker that comprises the amino acid sequence set forth in SEQ ID NO: 3. In certain embodiments, the variable regions are positioned from the N- to the C-terminus: V_{L}-V_{H}. In certain embodiments, the scFv is designated as “B10L5H”. In certain embodiments, the scFv comprises the amino acid sequence set forth in SEQ ID NO: 23. SEQ ID NO: 23 is provided below.

[SEQ ID NO: 23]
DIVMTQSPDSLAVSLGERATINCKSSQSVLDSYNNENNLAWYQQKPGQPP
KLLIYWASTRESGVPDRFSGSGSGTDFTLTISSLQAEDVAVYYCQQYTSE
PITFGQGTKVEIKGGGGSGGGGSGGGGSGGGSGGGGSEVQLLESGGGLVQ
PGGSLRLSCAASGFTFSDYQMSWVRQAPGKGLEWVSGIQGGGGSTYYADS
VKGRFTISRDNSKNTLYLQMNSLRAEDTAVYYCAREMWRGDYYSGMDVWG
QGTTVTVSS

In certain embodiments, the scFv comprises a V_{H} comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the V_{H} and V_{L} are linked via a linker that comprises the amino acid sequence set forth in SEQ ID NO: 4. In certain embodiments, the variable regions are positioned from the N- to the C-terminus: V_{H}-V_{L}. In certain embodiments, the scFv is designated as “B10H6L”. In certain embodiments, the scFv comprises the amino acid sequence set forth in SEQ ID NO: 24. SEQ ID NO: 24 is provided below.

[SEQ ID NO: 24]
EVQLLESGGGLVQPGGSLRLSCAASGFTFSDYQMSWVRQAPGKGLEWVS
GIQGGGGSTYYADSVKGRFTISRDNSKNTLYLQMNSLRAEDTAVYYCAR
EMWRGDYYSGMDVWGQGTTVTVSSGGGGSGGGGSGGGGSGGGGSGGGSG
GGGSDIVMTQSPDSLAVSLGERATINCKSSQSVLDSYNNENNLAWYQQK
PGQPPKLLIYWASTRESGVPDRFSGSGSGTDFTLTISSLQAEDVAVYYC
QQYTSEPITFGQGTKVEIK

In certain embodiments, the scFv comprises a V_{H} comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the V_{H} and V_{L} are linked via a linker that comprises the amino acid sequence set forth in SEQ ID NO: 4. In certain embodiments, the variable regions are positioned from the N- to the C-terminus: V_{L}-V_{H}. In certain embodiments, the scFv is designated as “B10L6H”. In certain embodiments, the scFv comprises the amino acid sequence set forth in SEQ ID NO: 25. SEQ ID NO: 25 is provided below.

[SEQ ID NO: 25]
DIVMTQSPDSLAVSLGERATINCKSSQSVLDSYNNENNLAWYQQKPGQP
PKLLIYWASTRESGVPDRFSGSGSGTDFTLTISSLQAEDVAVYYCQQYT
SEPITFGQGTKVEIKGGGGSGGGGSGGGGSGGGGSGGGSGGGGSEVQLL
ESGGGLVQPGGSLRLSCAASGFTFSDYQMSWVRQAPGKGLEWVSGIQGG
GGSTYYADSVKGRFTISRDNSKNTLYLQMNSLRAEDTAVYYCAREMWRG
DYYSGMDVWGQGTTVTVSS

In certain embodiments, the scFv comprises a V_{H} comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the V_{H} and V_{L} are linked via a linker that comprises the amino acid sequence set forth in SEQ ID NO: 5. In certain embodiments, the variable regions are positioned from the N- to the C-terminus: V_{H}-V_{L}. In certain embodiments, the scFv is designated as “B10H1L”. In certain embodiments, the scFv comprises the amino acid sequence set forth in SEQ ID NO: 26. SEQ ID NO: 26 is provided below.

[SEQ ID NO: 26]
EVQLLESGGGLVQPGGSLRLSCAASGFTFSDYQMSWVRQAPGKGLEWVS
GIQGGGGSTYYADSVKGRFTISRDNSKNTLYLQMNSLRAEDTAVYYCAR
EMWRGDYYSGMDVWGQGTTVTVSSGGGGSDIVMTQSPDSLAVSLGERAT
INCKSSQSVLDSYNNENNLAWYQQKPGQPPKLLIYWASTRESGVPDRFS
GSGSGTDFTLTISSLQAEDVAVYYCQQYTSEPITFGQGTKVEIK

In certain embodiments, the scFv comprises a V_{H} comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the V_{H} and V_{L} are linked via a linker that comprises the amino acid sequence set forth in SEQ ID NO: 5. In certain embodiments, the variable regions are positioned from the N- to the C-terminus: V_{L}-V_{H}. In certain embodiments, the scFv is designated as “B10L1H”. In certain embodiments, the scFv comprises the amino acid sequence set forth in SEQ ID NO: 27. SEQ ID NO: 27 is provided below.

[SEQ ID NO: 27]
DIVMTQSPDSLAVSLGERATINCKSSQSVLDSYNNENNLAWYQQKPGQP
PKLLIYWASTRESGVPDRFSGSGSGTDFTLTISSLQAEDVAVYYCQQYT
SEPITFGQGTKVEIKGGGGSEVQLLESGGGLVQPGGSLRLSCAASGFTF
SDYQMSWVRQAPGKGLEWVSGIQGGGGSTYYADSVKGRFTISRDNSKNT
LYLQMNSLRAEDTAVYYCAREMWRGDYYSGMDVWGQGTTVTVSS

In certain embodiments, the scFv comprises a V_{H} comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the V_{H} and V_{L} are linked via a linker that comprises the amino acid sequence set forth in SEQ ID NO: 6. In certain embodiments, the variable regions are positioned from the N- to the C-terminus: V_{H}-V_{L}. In certain embodiments, the scFv is designated as “B10H2L”. In certain embodiments, the scFv comprises the amino acid sequence set forth in SEQ ID NO: 28. SEQ ID NO: 28 is provided below.

[SEQ ID NO: 28]
EVQLLESGGGLVQPGGSLRLSCAASGFTFSDYQMSWVRQAPGKGLEWVS
GIQGGGGSTYYADSVKGRFTISRDNSKNTLYLQMNSLRAEDTAVYYCAR
EMWRGDYYSGMDVWGQGTTVTVSSGGGGSGGGGSDIVMTQSPDSLAVSL
GERATINCKSSQSVLDSYNNENNLAWYQQKPGQPPKLLIYWASTRESGV
PDRFSGSGSGTDFTLTISSLQAEDVAVYYCQQYTSEPITFGQGTKVEIK

In certain embodiments, the scFv comprises a V_{H} comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and a V_{L} comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17. In certain embodiments, the V_{H} and V_{L} are linked via a linker that comprises the amino acid sequence set forth in SEQ ID NO: 6. In certain embodiments, the variable regions are positioned from the N- to the C-terminus: V_{L}-V_{H}. In certain embodiments, the scFv is designated as “B10L2H”. In certain embodiments, the scFv comprises the amino acid sequence set forth in SEQ ID NO: 29. SEQ ID NO: 29 is provided below.

[SEQ ID NO: 29]
DIVMTQSPDSLAVSLGERATINCKSSQSVLDSYNNENNLAWYQQKPGQP
PKLLIYWASTRESGVPDRFSGSGSGTDFTLTISSLQAEDVAVYYCQQYT
SEPITFGQGTKVEIKGGGGSGGGGSEVQLLESGGGLVQPGGSLRLSCAA
SGFTFSDYQMSWVRQAPGKGLEWVSGIQGGGGSTYYADSVKGRFTISRD
NSKNTLYLQMNSLRAEDTAVYYCAREMWRGDYYSGMDVWGQGTTVTVSS

In certain embodiments, the extracellular antigen-binding domain binds to mesothelin. In certain embodiments, the extracellular antigen-binding domain binds to human mesothelin).

In certain embodiments, the V_{H} comprises an amino acid sequence that is at least about 80% (e.g., at least about 85%, at least about 90%, at least about 95%, at least about 99%, or about 100%) identical or homologous to the amino acid sequence set forth in SEQ ID NO: 17. For example, the V_{H} comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical or homologous to SEQ ID NO: 30. In certain embodiments, the V_{H} comprises the amino acid sequence set forth in SEQ ID NO: 30. SEQ ID NO: 30 is provided below.

[SEQ ID NO: 30]
QVELVQSGAEVKKPGESLKISCKGSGYSFTSYWIGWVRQAPGKGLEWMG
IIDPGDSRTRYSPSFQGQVTISADKSISTAYLQWSSLKASDTAMYYCAR
GQLYGGTYMDGWGQGTLVTVSS

In certain embodiments, the V_{L} comprises an amino acid sequence that is at least about 80% (e.g., at least about 85%, at least about 90%, at least about 95%, at least about 99%, or about 100%) identical or homologous to the amino acid sequence set forth in SEQ ID NO: 18. For example, the V_{L} comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical or homologous to SEQ ID NO: 31. In certain embodiments, the V_{L} comprises the amino acid sequence set forth in SEQ ID NO: 31. SEQ ID NO: 31 is provided below.

[SEQ ID NO: 31]
ALTQPASVSGSPGQSITISCTGTSSDIGGYNSVSWYQQHPGKAPKLMIY
GVNNRPSGVSNRFSKSGNTASLTISGLQAEDEADYYCSSYDIESATPVF
GGGTKLTVLGQ

In certain embodiments, the V_{H} comprises the amino acid sequence set forth in SEQ ID NO: 30, and the V_{L} comprises the amino acid sequence set forth in SEQ ID NO: 31.

In certain embodiments, the V_{H} comprises a CDR1, a CDR2, and a CDR3 of the V_{H} sequence set forth in SEQ ID NO: 30.

In certain embodiments, the V_{L} comprises a CDR1, a CDR2, and a CDR3 of the V_{L} sequence set forth in SEQ ID NO: 31.

In certain embodiments, the V_{H} and V_{L} are linked via a linker. In certain embodiments, the linker comprises the amino acid sequence set forth in SEQ ID NO: 2.

As used herein, the term “a conservative sequence modification” refers to an amino acid modification that does not significantly affect or alter the binding characteristics of the extracellular antigen-binding domain (e.g., an scFv). Conservative modifications can include amino acid substitutions, additions and deletions. Modifications can be introduced into the extracellular antigen-binding domain of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function using the functional assays described herein. In certain embodiments, no more than one, no more than two, no more than three, no more than four, no more than five residues within a specified sequence or a CDR region are altered.

The V_{H} and/or V_{L} amino acid sequences having at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) identity or homology to a specific sequence (e.g., SEQ ID NO: 10, SEQ ID NO: 11, SEQ ID NO: 30, or SEQ ID NO: 31) may contain substitutions (e.g., conservative substitutions), insertions, or deletions relative to the specified sequence(s), but retain the ability to bind to the target antigen. In certain embodiments, a total of 1 to 10 amino acids are substituted, inserted and/or deleted in a specific sequence (e.g., SEQ ID NO: 10, SEQ ID NO: 11, SEQ ID NO: 30, or SEQ ID NO: 31). In certain embodiments, substitutions, insertions, or deletions occur in regions outside the CDRs (e.g., in the FRs) of the extracellular antigen-binding domain. In certain embodiments, the extracellular antigen-binding domain comprises V_{H} and/or V_{L} sequence selected from SEQ ID NOs: 10, 11, 30, and 31, including post-translational modifications of that sequence (SEQ ID NOs: 10, 11, 30, and 31).

In addition, the extracellular antigen-binding domain can comprise a leader or a signal peptide that directs the nascent protein into the endoplasmic reticulum. Signal peptide or leader can be helpful if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the N-terminus of the extracellular antigen-binding domain.

In certain embodiments, the signal peptide is an IL-2 signal sequence. In certain embodiments, the signal peptide is a human IL-2 signal sequence. In certain embodiments, the signal peptide comprises or consists of the amino acid sequence set forth in SEQ ID NO: 32. In certain embodiments, the signal peptide is a mouse IL-2 signal sequence. In certain embodiments, the signal peptide comprises or consists of the amino acid sequence set forth in SEQ ID NO: 33. SEQ ID Nos: 32 and 33 are provided below.

[SEQ ID NO: 32]
MYRMQLLSCIALSLALVINS
[SEQ ID NO: 33]
MYSMQLASCVTLTLVLLVNS

In certain embodiments, the signal peptide is a kappa signal sequence. In certain embodiments, the signal peptide is a human kappa signal sequence. In certain embodiments, the signal peptide comprises or consists of the amino acid sequence set forth in SEQ ID NO: 34. In certain embodiments, the signal peptide is a mouse kappa signal sequence. In certain embodiments, the signal peptide comprises or consists of the amino acid sequence set forth in SEQ ID NO: 35. SEQ ID Nos: 34 and 35 are provided below.

[SEQ ID NO: 34]
METPAQLLFLLLLWLPDTTG
[SEQ ID NO: 35]
METDTLLLWVLLLWVPGSTG

In certain embodiments, the signal peptide is a CD8 signal sequence. In certain embodiments, the signal peptide is a human CD8 signal sequence. In certain embodiments, the signal peptide comprises or consists of the amino acid sequence set forth in SEQ ID NO: 36. In certain embodiments, the signal peptide is a truncated human CD8 signal sequence. In certain embodiments, the signal peptide comprises or consists of the amino acid sequence set forth in SEQ ID NO: 37. SEQ ID Nos: 36 and 37 are provided below.

[SEQ ID NO: 36]
MALPVTALLLPLALLLHAARP
[SEQ ID NO: 37]
MALPVTALLLPLALLLHA

In certain embodiments, the signal peptide is an albumin signal sequence. In certain embodiments, the signal peptide is a human albumin signal sequence. In certain embodiments, the signal peptide comprises or consists of the amino acid sequence set forth in SEQ ID NO: 38. SEQ ID NO: 38 is provided below.

[SEQ ID NO: 38]
MKWVTFISLLFSSAYS

In certain embodiments, the signal peptide is a prolactin signal sequence. In certain embodiments, the signal peptide is a human prolactin signal sequence. In certain embodiments, the signal peptide comprises or consists of the amino acid sequence set forth in SEQ ID NO: 39. SEQ ID NO: 39 is provided below.

[SEQ ID NO: 39]
MDSKGSSQKGSRLLLLLVVSNLLLCQGVVS

### 5.3.2.2. Transmembrane Domain of a CAR

In certain embodiments, the transmembrane domain comprises a hydrophobic alpha helix that spans at least a portion of the membrane. Different transmembrane domains result in different receptor stability. After antigen recognition, receptors cluster and a signal are transmitted to the cell. In accordance with the presently disclosed subject matter, the transmembrane domain can comprise a native or modified transmembrane domain of CD8 or a fragment thereof, a native or modified transmembrane domain of CD28 or a fragment thereof, a native or modified transmembrane domain of CD32 or a fragment thereof, a native or modified transmembrane domain of CD4 or a fragment thereof, a native or modified transmembrane domain of 4-1BB or a fragment thereof, a native or modified transmembrane domain of OX40 or a fragment thereof, a native or modified transmembrane domain of ICOS or a fragment thereof, a native or modified transmembrane domain of CD84 or a fragment thereof, a native or modified transmembrane domain of CD166 or a fragment thereof, a native or modified transmembrane domain of CD8a or a fragment thereof, a native or modified transmembrane domain of CD8b or a fragment thereof, a native or modified transmembrane domain of ICAM-1 or a fragment thereof, a native or modified transmembrane domain of CTLA-4 or a fragment thereof, a native or modified transmembrane domain of CD27 or a fragment thereof, a native or modified transmembrane domain of CD40 or a fragment thereof, NKGD2 or a fragment thereof, or a combination thereof.

In certain embodiments, the transmembrane domain comprises a CD8 polypeptide (e.g., a transmembrane domain of CD8 or a fragment thereof).

In certain embodiments, the transmembrane domain comprises a CD8 polypeptide that is a transmembrane domain of human CD8 or a fragment thereof. In certain embodiments, the CD8 polypeptide comprises or consists of an amino acid sequence that is at least about 85%, about 90%, about 95%, about 96%, about 97%, about 98%, about 99% or about 100% identical or homologous to the amino acid sequence having a Uniprot Reference No: P01732-1 (SEQ ID NO: 40) or a fragments thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 40, which is at least 20, or at least 30, or at least 40, or at least 50, and up to 235 amino acids in length. In certain embodiments, the CD8 polypeptide comprises or consists of an amino acid sequence of amino acids 1 to 235, 1 to 50, 50 to 100, 100 to 150, 150 to 200, 137 to 209, 183 to 203, 204 to 235, or 200 to 235 of SEQ ID NO: 27. In certain embodiments, the transmembrane domain comprises a CD8 polypeptide comprising or consisting of amino acids 137 to 209 of SEQ ID NO: 40. In certain embodiments, the transmembrane domain comprises a CD8 polypeptide comprising or consisting of amino acids 183 to 203 of SEQ ID NO: 40. SEQ ID NO: 40 is provided below

[SEQ ID NO: 40]
MALPVTALLLPLALLLHAARPSQFRVSPLDRTWNLGETVELKCQVLLSN
PTSGCSWLFQPRGAAASPTFLLYLSQNKPKAAEGLDTQRFSGKRLGDTF
VLTLSDFRRENEGYYFCSALSNSIMYFSHFVPVFLPAKPTTTPAPRPPT
PAPTIASQPLSLRPEACRPAAGGAVHTRGLDFACDIYIWAPLAGTCGVL
LLSLVITLYCNHRNRRRVCKCPRPVVKSGDKPSLSARYV

In certain embodiments, the transmembrane domain comprises a CD8 polypeptide that is a transmembrane domain of mouse CD8 or a fragment thereof. In certain embodiments, the CD8 polypeptide comprises or consists of an amino acid sequence that is at least about 85%, about 90%, about 95%, about 96%, about 97%, about 98%, about 99% or about 100% identical or homologous to the amino acid sequence having a NCBI Reference No: AAA92533.1 (SEQ ID NO: 41) or a fragment thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 41, which is at least about 20, or at least about 30, or at least about 40, or at least about 50, or at least about 60, or at least about 70, or at least about 100, or at least about 200, and up to 247 amino acids in length. In certain embodiments, the CD8 polypeptide comprises or consists of an amino acid sequence of amino acids 1 to 247, Ito 50, 50 to 100, 100 to 150, 150 to 200, 151 to 219, 197 to 217, or 200 to 247 of SEQ ID NO: 41. In certain embodiments, the transmembrane domain comprises a CD8 polypeptide comprising or consisting of amino acids 151 to 219 of SEQ ID NO: 41. In certain embodiments, the transmembrane domain comprises a CD8 polypeptide comprising or consisting of amino acids 197 to 217 of SEQ ID NO: 41. SEQ ID NO: 41 is provided below.

[SEQ ID NO: 41]
MASPLTRFLSLNLLLMGESIILGSGEAKPQAPELRIFPKKMDAELGQKV
DLVCEVLGSVSQGCSWLFQNSSSKLPQPTFVVYMASSHNKITWDEKLNS
SKLFSAVRDTNNKYVLTLNKFSKENEGYYFCSVISNSVMYFSSVVPVLQ
KVNSTTTKPVLRTPSPVHPTGTSQPQRPEDCRPRGSVKGTGLDFACDIY
IWAPLAGICVAPLLSLIITLICYHRSRKRVCKCPRPLVRQEGKPRPSEK
IV

In certain embodiments, the transmembrane domain of a presently disclosed CAR comprises a CD28 polypeptide (e.g., a transmembrane domain of CD28 or a fragment thereof).

In certain embodiments, the transmembrane domain comprises a CD28 polypeptide that is a transmembrane domain of human CD28 or a fragment thereof. In certain embodiments, the CD28 polypeptide comprises or consists of an amino acid sequence that is at least about 85%, about 90%, about 95%, about 96%, about 97%, about 98%, about 99% or 100% identical or homologous to the amino acid sequence having a NCBI Reference No: NP_006130 (SEQ ID No: 42) or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 42, which is at least 20, or at least 30, or at least 40, or at least 50, and up to 220 amino acids in length. In certain embodiments, the CD28 polypeptide comprises or consists of an amino acid sequence of amino acids 1 to 220, 1 to 50, 50 to 100, 100 to 150, 150 to 200, 153 to 179, or 200 to 220 of SEQ ID NO: 42. In certain embodiments, the transmembrane domain comprises a CD28 polypeptide comprising or consisting of amino acids 153 to 179 of SEQ ID NO: 42. SEQ ID NO: 42 is provided below.

[SEQ ID NO: 42]
MLRLLLALNLFPSIQVTGNKILVKQSPMLVAYDNAVNLSCKYSYNLFSRE
FRASLHKGLDSAVEVCVVYGNYSQQLQVYSKTGFNCDGKLGNESVTFYLQ
NLYVNQTDIYFCKIEVMYPPPYLDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRS

An exemplary nucleotide sequence encoding amino acid 153 to 179 of SEQ ID NO: 42 is set forth in SEQ ID NO: 43, which is provided below.

[SEQ ID NO: 43]
TTTTGGGTGCTGGTGGTGGTTGGTGGAGTCCTGGCTTGCTATAGCTTGCT
AGTAACAGTGGCCTTTATTATTTTCTGGGTG

In certain embodiments, the transmembrane domain comprises a CD28 polypeptide that is a transmembrane domain of mouse CD28 or a fragment thereof. In certain embodiments, the CD28 polypeptide comprises or consists of an amino acid sequence that is at least about 85%, about 90%, about 95%, about 96%, about 97%, about 98%, about 99% or 100% identical or homologous to the amino acid sequence having a NCBI Reference No: NP 031668.3 (SEQ ID No: 44) or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 44, which is at least 20, or at least 30, or at least 40, or at least 50, and up to 218 amino acids in length. Alternatively or additionally, in certain embodiments, the CD28 polypeptide comprises or consists of an amino acid sequence of amino acids 1 to 220, 1 to 50, 50 to 100, 100 to 150, 150 to 200, 151 to 177, or 200 to 218 of SEQ ID NO: 44. In certain embodiments, the transmembrane domain comprises a CD28 polypeptide comprising or consisting of amino acids 151 to 177 of SEQ ID NO: 44. SEQ ID NO: 44 is provided below.

[SEQ ID NO: 44]
MTLRLLFLALNFFSVQVTENKILVKQSPLLVVDSNEVSLSCRYSYNLLAK
EFRASLYKGVNSDVEVCVGNGNFTYQPQFRSNAEFNCDGDFDNETVTFRL
WNLHVNHTDIYFCKIEFMYPPPYLDNERSNGTIIHIKEKHLCHTQSSPKL
FWALVVVAGVLFCYGLLVTVALCVIWTNSRRNRLLQSDYMNMTPRRPGLT
RKPYQPYAPARDFAAYRP

In certain embodiments, the CAR further comprises a hinge/spacer region that links the extracellular antigen-binding domain to the transmembrane domain. The hinge/spacer region can be flexible enough to allow the antigen binding domain to orient in different directions to facilitate antigen recognition while preserving the activating activity of the CAR.

In certain embodiments, the hinge/spacer region of the CAR comprises a native or modified hinge region of CD8 or a fragment thereof, a native or modified hinge region of CD28 or a fragment thereof, a native or modified hinge region of CD35 or a fragment thereof, a native or modified hinge region of CD40 or a fragment thereof, a native or modified hinge region of 4-1BB or a fragment thereof, a native or modified hinge region of OX40 or a fragment thereof, a native or modified hinge region of CD84 or a fragment thereof, a native or modified hinge region of CD 166 or a fragment thereof, a native or modified hinge region of CD8a or a fragment thereof, a native or modified hinge region of CD8b or a fragment thereof, a native or modified hinge region of ICOS or a fragment thereof, a native or modified hinge region of ICAM-1 or a fragment thereof, a native or modified hinge region of CTLA-4 or a fragment thereof, a native or modified hinge region of CD27 or a fragment thereof, a native or modified hinge region of CD40 or a fragment thereof, a native or modified hinge region of NKGD2 or a fragment thereof, a synthetic polypeptide (not based on a protein associated with the immune response), or a combination thereof. The hinge/spacer region can be the hinge region from IgG1, or the CH2CH3 region of immunoglobulin and portions of CD3, a portion of a CD28 polypeptide (e.g., a portion of SEQ ID NO: 42 or 44), a portion of a CD8 polypeptide (e.g., a portion of SEQ ID NO: 40 or 41), a variation of any of the foregoing which is at least about 80%, at least about 85%, at least about 90%, at least about 95%, or at least about 100% homologous or identical thereto, or a synthetic spacer sequence.

In certain embodiments, the transmembrane domain comprises a CD28 hinge/spacer region. In certain embodiments, the CD28 hinge/spacer region comprises amino acids 114 to 152 of SEQ ID NO: 42.

### 5.3.2.3. Intracellular Domain of a CAR

In certain embodiments, the CAR comprises an intracellular domain. In certain embodiments, the intracellular domain of the CAR comprises a CD32 polypeptide. CD32 can activate or stimulate a cell (e.g., a cell of the lymphoid lineage, e.g., a T cell). Wild type (“native”) CD35 comprises three functional immunoreceptor tyrosine-based activation motifs (ITAMs), three functional basic-rich stretch (BRS) regions (BRS1, BRS2 and BRS3). CD3ζ transmits an activation signal to the cell (e.g., a cell of the lymphoid lineage, e.g., a T cell) after antigen is bound. The intracellular signaling domain of the CD32-chain is the primary transmitter of signals from endogenous TCRs.

In certain embodiments, the intracellular domain comprises a native CD3ζ. In certain embodiments, the CD3ζ polypeptide comprises or consists of an amino acid sequence that is at least about 85%, about 90%, about 95%, about 96%, about 97%, about 98%, about 99% or about 100% homologous or identical to the amino acid sequence having a NCBI Reference No: NP 932170 (SEQ ID NO: 45) or a fragment thereof, and/or may optionally comprise up to one or up to two or up to three conservative amino acid substitutions. In certain embodiments, the CD3ζ polypeptide comprises or consists of an amino acid sequence that is a consecutive portion of SEQ ID NO: 45, which is at least 20, or at least 30, or at least 40, or at least 50, and up to 164 amino acids in length. In certain embodiments, the CD35 polypeptide comprises or consists of an amino acid sequence of amino acids 1 to 164, 1 to 50, 50 to 100, 52 to 164, 100 to 150, or 150 to 164 of SEQ ID NO: 45. In certain embodiments, the intracellular domain comprises a CD32 polypeptide comprising or consisting of amino acids 52 to 164 of SEQ ID NO: 45. SEQ ID NO: 45 is provided below.

[SEQ ID NO: 45]
MKWKALFTAAILQAQLPITEAQSFGLLDPKLCYLLDGILFIYGVILTALF
LRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATK
DTYDALHMQALPPR

In certain embodiments, the intracellular signaling domain comprises a CD3ζ polypeptide comprising or consisting of the amino acid sequence set forth in SEQ ID NO: 46. SEQ ID NO: 46 is provided below.

[SEQ ID NO: 46]
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

An exemplary nucleotide sequence encoding the amino acid sequence of SEQ ID NO: 46 is set forth in SEQ ID NO: 47, which is as provided below.

[SEQ ID NO: 47]
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGC

In certain embodiments, the intracellular signaling domain further comprises at least a co-stimulatory signaling region. In certain embodiments, the co-stimulatory signaling region comprises at least one co-stimulatory molecule or a fragment thereof. In certain embodiments, the co-stimulatory signaling region comprises an intracellular domain of at least one co-stimulatory molecule or a fragment thereof.

As used herein, a “co-stimulatory molecule” refers to a cell surface molecule other than antigen receptor or its ligand that can provide an efficient response of lymphocytes to an antigen. In certain embodiments, a co-stimulatory molecule can provide optimal lymphocyte activation. Non-limiting examples of co-stimulatory molecules include CD28, 4-1BB, OX40, ICOS, DAP-10, CD27, CD40, NKGD2, CD2, FN14, HVEM, LTβR, CD28H, TNFR1, TNFR2, BAFF-R, BCMA, TACI, TROY, RANK, CD40, CD27, CD30, ED AR, XEDAR, GITR, DR6, and NGFR, and combinations thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen recognizing receptor (e.g., a CAR) binds to its target antigen. As one example, a 4-1BB ligand (i.e., 4-1BBL) may bind to 4-1BB for providing an intracellular signal that in combination with a CAR signal induces an effector cell function of the CAR⁺ T cell.

In certain embodiments, the intracellular signaling domain comprises a co-stimulatory signaling region that comprises a CD28 polypeptide, e.g., an intracellular domain of CD28 or a fragment thereof. In certain embodiments, the co-stimulatory signaling region comprises an intracellular domain of human CD28 or a fragment thereof. In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% identical or homologous to the amino acid sequence set forth in SEQ ID NO: 42 or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 42, which is at least 20, or at least 30, or at least 40, or at least 50, and up to 220 amino acids in length. In certain embodiments, the CD28 polypeptide comprises or consists of an amino acid sequence of amino acids 1 to 220, 1 to 50, 50 to 100, 100 to 150, 114 to 220, 150 to 200, 180 to 220, or 200 to 220 of SEQ ID NO: 42. In certain embodiments, the intracellular signaling domain comprises a co-stimulatory signaling region that comprises a CD28 polypeptide comprising or consisting of an amino acid sequence of amino acids 180 to 220 of SEQ ID NO: 42.

An exemplary nucleic acid sequence encoding amino acids 180 to 220 of SEQ ID NO: 42 is set forth in SEQ ID NO: 48, which is provided below.

[SEQ ID NO: 48]
AGGAGTAAGAGGAGCAGGCTCCTGCACAGTGACTACATGAACATGACTCC
CCGCCGCCCCGGGCCCACCCGCAAGCATTACCAGCCCTATGCCCCACCAC
GCGACTTCGCAGCCTATCGCTCC

In certain embodiments, the co-stimulatory signaling region comprises an intracellular domain of mouse CD28 or a fragment thereof. In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% identical or homologous to the amino acid sequence set forth in SEQ ID NO: 44 or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 44 which is at least about 20, or at least about 30, or at least about 40, or at least about 50, and up to 218 amino acids in length. In certain embodiments, the CD28 polypeptide comprises or consists of an amino acid sequence of amino acids 1 to 218, 1 to 50, 50 to 100, 100 to 150, 150 to 218, 178 to 218, or 200 to 218 of SEQ ID NO: 44. In certain embodiments, the co-stimulatory signaling region of a presently disclosed CAR comprises a CD28 polypeptide that comprises or consists of the amino acids 178 to 218 of SEQ ID NO: 44.

In certain embodiments, the intracellular signaling domain comprises a co-stimulatory signaling region that comprises a 4-1BB polypeptide, e.g., an intracellular domain of 4-1BB or a fragment thereof. In certain embodiments, the co-stimulatory signaling region comprises an intracellular domain of human 4-1BB or a fragment thereof. In certain embodiments, the 4-1BB polypeptide comprises or consists of an amino acid sequence that is at least about 80%, at least about 85%, at least about 90%, at least about 95%, at least about 96%, at least about 97%, at least about 98%, or at least about 99%, at least about 100% identical or homologous to the amino acid sequence having a NCBI Ref. No.: NP_001552 (SEQ ID NO: 49) or a fragment thereof, and/or may optionally comprise up to one or up to two or up to three conservative amino acid substitutions. In embodiments, the 4-1BB polypeptide comprises or consists of an amino acid sequence that is a consecutive portion of SEQ ID NO: 49, which is at least 20, or at least 30, or at least 40, or at least 50, or at least 100, or at least 150, or at least 150, and up to 255 amino acids in length. In certain embodiments, the 4-1BB polypeptide comprises or consists of an amino acid sequence of amino acids 1 to 255, 1 to 50, 50 to 100, 100 to 150, 150 to 200, or 200 to 255 of SEQ ID NO: 49. In certain embodiments, the intracellular signaling domain of the CAR comprises a co-stimulatory signaling region that comprises a 4-IBB polypeptide comprising or consisting of an amino acid sequence of amino acids 214 to 255 of SEQ ID NO: 49. SEQ ID NO: 49 is provided below.

[SEQ ID NO: 49]
MGNSCYNIVATLLLVLNFERTRSLQDPCSNCPAGTFCDNNRNQICSPCPP
NSFSSAGGQRTCDICRQCKGVFRTRKECSSTSNAECDCTPGFHCLGAGCS
MCEQDCKQGQELTKKGCKDCCFGTFNDQKRGICRPWTNCSLDGKSVLVNG
TKERDVVCGPSPADLSPGASSVTPPAPAREPGHSPQIISFFLALTSTALL
FLLFFLTLRFSVVKRGRKKLLYIFKQPFMRPVQTTQEEDGCSCRFPEEEE
GGCEL

In certain embodiments, the intracellular signaling domain of the CAR comprises a co-stimulatory signaling region that comprises intracellular domains of two or more costimulatory molecules or portions thereof, e.g., an intracellular domain of CD28 or a fragment thereof and an intracellular domain of 4-1BB or a fragment thereof, or an intracellular domain of CD28 or a fragment thereof and an intracellular domain of OX40 or a fragment thereof.

### 5.3.2.4. Exemplified CARs

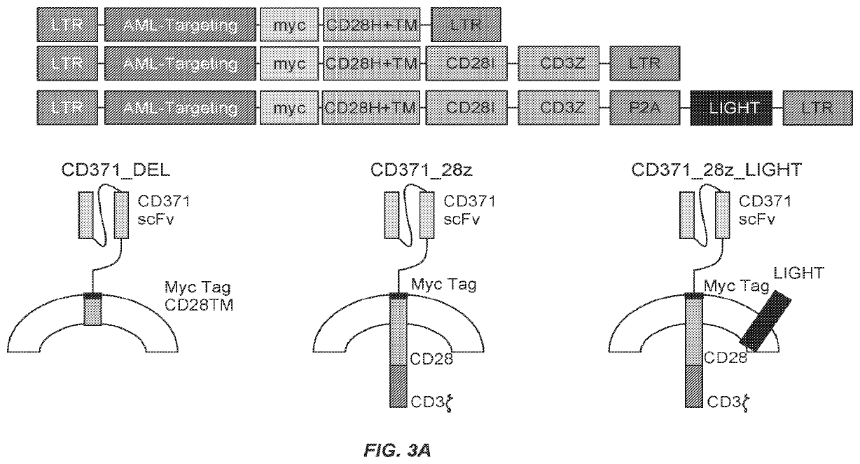

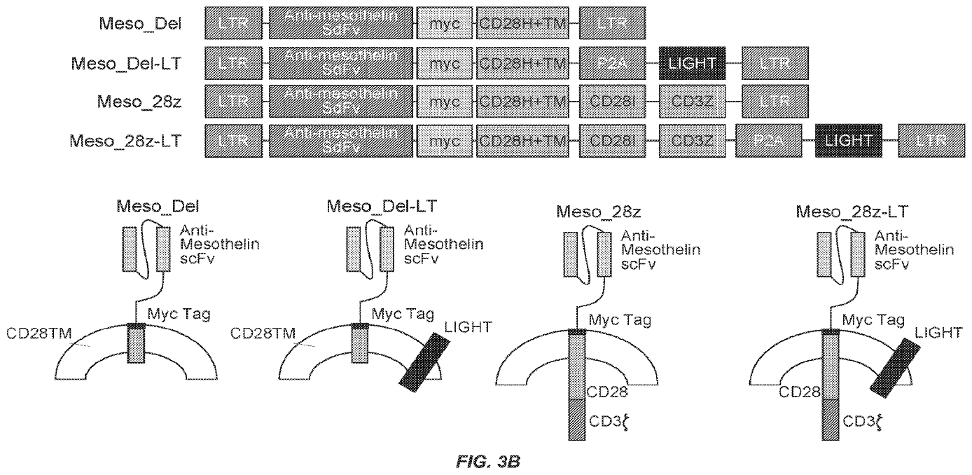

In certain embodiments, the CAR is a CD371-targeted CAR. In certain embodiments, the CAR comprises (a) an extracellular antigen-binding domain comprising (i) a V_{H} that comprises a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14, and (ii) a V_{L} that comprises a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide (e.g., an intracellular domain of human CD28 or a fragment thereof). In certain embodiments, the V_{H} comprises the amino acid sequence set forth in SEQ ID NO: 10. In certain embodiments, the V_{L} comprises the amino acid sequence set forth in SEQ ID NO: 11. In certain embodiments, the transmembrane domain comprises a CD28 polypeptide comprising amino acids 153 to 179 of SEQ ID NO: 42. In certain embodiments, the intracellular signaling domain comprises a CD3ζ polypeptide comprising the amino acid sequence set forth in SEQ ID NO: 46. In certain embodiments, the co-stimulatory signaling region comprises a CD28 polypeptide comprising amino acids 180 to 220 of SEQ ID NO: 42. In certain embodiments, the V_{H} and V_{L} are linked via a linker comprising or consisting of the amino acid sequence set forth in SEQ ID NO: 1. In certain embodiments, the V_{H} and V_{L} are positioned from the N- to the C-terminus: V_{H}-V_{L}. In certain embodiments, the CAR is designated as “CD371_28z”, “B10H4L”, “B10-28z”, “B10HL”, “B10HL28z”, or “B10H4L28z”. Schematics of construct maps is illustrated in FIG. 3A. In certain embodiments, the CAR comprises an amino acid sequence that is at least about 85%, about 90%, about 95%, about 96%, about 97%, about 98%, about 99% or about 100% identical to the amino acid sequence set forth in SEQ ID NO: 50. In certain embodiments, the CAR comprises an amino acid sequence set forth in SEQ ID NO: 50, which is provided below.

[SEQ ID NO: 50]
MALPVTALLLPLALLLHAEVQLLESGGGLVQPGGSLRLSCAASGFTFSDY
QMSWVRQAPGKGLEWVSGIQGGGGSTYYADSVKGRFTISRDNSKNTLYLQ
MNSLRAEDTAVYYCAREMWRGDYYSGMDVWGQGTTVTVSSGGGGSGGGGS
GGGSGGGGSDIVMTQSPDSLAVSLGERATINCKSSQSVLDSYNNENNLAW
YQQKPGQPPKLLIYWASTRESGVPDRFSGSGSGTDFTLTISSLQAEDVAV
YYCQQYTSEPITFGQGTKVEIKEQKLISEEDLAAAIEVMYPPPYLDNEKS
NGTIIHVKGKHLCPSPLFPGPSKPFWVLVVVGGVLACYSLLVTVAFIIFW
VRSKRSRLLHSDYMNMTPRRPGPTRKHYQPYAPPRDFAAYRSRVKFSRSA
DAPAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQA
LPPR

In certain embodiments, the CAR is a mesothelin-targeted CAR. In certain embodiments, the CAR comprises (a) an extracellular antigen-binding domain comprising a V_{H} that comprises the amino acid sequence set forth in SEQ ID NO: 30, and (ii) a V_{L} that comprises the amino acid sequence set forth in SEQ ID NO: 31; (b) a transmembrane domain comprising a CD28 polypeptide (e.g., a transmembrane domain of human CD28 or a fragment thereof), and (c) an intracellular signaling domain comprising (i) a CD32 polypeptide, and (ii) a co-stimulatory signaling region comprising a CD28 polypeptide (e.g., an intracellular domain of human CD28 or a fragment thereof). In certain embodiments, the transmembrane domain comprises a CD28 polypeptide comprising amino acids 153 to 179 of SEQ ID NO: 42. In certain embodiments, the intracellular signaling domain comprises a CD3ζ polypeptide comprising the amino acid sequence set forth in SEQ ID NO: 46. In certain embodiments, the co-stimulatory signaling region comprises a CD28 polypeptide comprising amino acids 180 to 220 of SEQ ID NO: 42. In certain embodiments, the CAR is designated as “hMSLN28z” or “Meso_28z”. Schematics of construct maps is illustrated in FIG. 3B. In certain embodiments, the CAR comprises an amino acid sequence that is at least about 85%, about 90%, about 95%, about 96%, about 97%, about 98%, about 99% or about 100% homologous to the amino acid sequence set forth in SEQ ID NO: 51, which is provided below. In certain embodiments, the CAR comprises an amino acid sequence set forth in SEQ ID NO: 51, which is provided below.

[SEQ ID NO: 51]
MALPVTALLLPLALLLHAQVELVQSGAEVKKPGESLKISCKGSGYSFTSY
WIGWVRQAPGKGLEWMGIIDPGDSRTRYSPSFQGQVTISADKSISTAYLQ
WSSLKASDTAMYYCARGQLYGGTYMDGWGQGTLVTVSSGGGGSGGGGSGG
GGSDIALTQPASVSGSPGQSITISCTGTSSDIGGYNSVSWYQQHPGKAPK
LMIYGVNNRPSGVSNRFSGSKSGNTASLTISGLQAEDEADYYCSSYDIES
ATPVFGGGTKLTVLGQEQKLISEEDLAAAIEVMYPPPYLDNEKSNGTIIH
VKGKHLCPSPLFPGPSKPFWVLVVVGGVLACYSLLVTVAFIIFWVRSKRS
RLLHSDYMNMTPRRPGPTRKHYQPYAPPRDFAAYRSRVKFSRSADAPAYQ
QGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR

### 5.3.3. T-Cell Receptor (TCR)

In certain embodiments, the antigen-recognizing receptor is a T-cell receptor (TCR). A TCR is a disulfide-linked heterodimeric protein consisting of two variable chains expressed as part of a complex with the invariant CD3 chain molecules. A TCR found on the surface of T cells is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains: Variable (V) region and a Constant (C) region. The Constant region is proximal to the cell membrane, followed by a transmembrane region and a short cytoplasmic tail. The Variable region binds to the peptide/MHC complex. The variable domain of both chains each has three complementarity determining regions (CDRs).

In certain embodiments, a TCR can form a receptor complex with three dimeric signaling modules CD36/c, CD3y/8 and CD247 ζ/ζ or ζ/η. When a TCR complex engages with its antigen and MHC (peptide/MHC), the T cell expressing the TCR complex is activated.

In certain embodiments, the TCR is an endogenous TCR. In certain embodiments, the antigen-recognizing receptor is naturally occurring TCR.

In certain embodiments, the antigen-recognizing receptor is an exogenous TCR. In certain embodiments, the antigen-recognizing receptor is a recombinant TCR. In certain embodiments, the antigen-recognizing receptor is a non-naturally occurring TCR. In certain embodiments, the non-naturally occurring TCR differs from any naturally occurring TCR by at least one amino acid residue. In certain embodiments, the non-naturally occurring TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the non-naturally occurring TCR is modified from a naturally occurring TCR by at least one amino acid residue. In certain embodiments, the non-naturally occurring TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

### 5.3.4. TCR Like Fusion Molecules

In certain embodiments, the antigen-recognizing receptor is a TCR like fusion molecule. Non-limiting examples of TCR fusion molecules include HLA-Independent TCR-based Chimeric Antigen Receptor (also known as “HIT-CAR”, e.g., those disclosed in International Patent Application No. PCT/US19/017525, which is incorporated by reference in its entirety), and T cell receptor fusion constructs (TRuCs) (e.g., those disclosed in Baeuerle et al., “Synthetic TRUC receptors engaging the complete T cell receptor for potent anti-turn or response,” Nature Communications volume 10, Article number: 2087 (2019), which is incorporated by reference in its entirety).

In certain embodiments, the TCR like fusion molecule comprises an antigen binding chain that comprises an extracellular antigen-binding domain and a constant domain, wherein the TCR like fusion molecule binds to an antigen in an HLA-independent manner. In certain embodiments, the constant domain comprises a T cell receptor constant region selected from the group consisting of a native or modified TRAC peptide, a native or modified TRBC peptide, a native or modified TRDC peptide, a native or modified TRGC peptide and any variants or functional fragments thereof. In certain embodiments, the constant domain comprises a native or modified TRAC peptide. In certain embodiments, the constant domain comprises a native or modified TRBC peptide. In certain embodiments, the constant domain is capable of forming a homodimer or a heterodimer with another constant domain. In certain embodiments, the antigen binding chain is capable of associating with a CD3ζ polypeptide. In certain embodiments, the antigen binding chain, upon binding to an antigen, is capable of activating the CD3ζ polypeptide associated to the antigen binding chain. In certain embodiments, the activation of the CD3ζ polypeptide is capable of activating an immunoresponsive cell. In certain embodiments, the TCR like fusion molecule is capable of integrating with a CD3 complex and providing HLA-independent antigen recognition. In certain embodiments, the TCR like fusion molecule replaces an endogenous TCR in a CD3/TCR complex. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a ligand for a cell-surface receptor, a receptor for a cell surface ligand, an antigen binding portion of an antibody or a fragment thereof or an antigen binding portion of a TCR. In certain embodiments, the extracellular antigen-binding domain of the TCR like fusion molecule comprises one or two immunoglobulin variable region(s). In certain embodiments, the extracellular antigen-binding domain of the TCR like fusion molecule comprises a heavy chain variable region (V_{H}) of an antibody. In certain embodiments, the extracellular antigen-binding domain of the TCR like fusion molecule comprises a light chain variable region (V_{L}) of an antibody. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a V_{H} of an antibody, wherein the V_{H} is capable of dimerizing with another extracellular antigen-binding domain comprising a V_{L} of the antibody and form a fragment variable (Fv). In certain embodiments, the extracellular antigen-binding domain of the TCR like fusion molecule comprises a V_{L} of an antibody, wherein the V_{L} is capable of dimerizing with another extracellular antigen-binding domain comprising a V_{H} of the antibody and form a fragment variable (Fv).

The TCR like fusion molecule can bind to a tumor antigen or a pathogen antigen. In certain embodiments, the TCR like fusion molecule binds to a tumor antigen.

### 5.4 Cells

The presently disclosed subject matter provides engineered immunoresponsive cells over-expressing LIGHT. The engineered immunoresponsive cells comprise an antigen recognizing receptor, e.g., one disclosed in Section 5.3. LIGHT overexpression of engineered immunoresponsive cells (e.g., engineered T cells, e.g., CAR-T cells) can enhance the anti-tumor effect and costimulatory potential of the cells. The presently disclosed LIGHT-overexpressing immunoresponsive cells have unique biological properties that not only modulate the immunogenicity of the target cells (e.g., tumor cells) through LIGHT-LTβR interaction but also provide immunostimulatory properties to the immunoresponsive cells and other immune effectors through LIGHT-HVEM interaction. The presently disclosed LIGHT-overexpressing immunoresponsive cells can modify the tumor microenvironment (TME) and induce an anti-tumor response by endogenous immune effectors.

The presently disclosed LIGHT-overexpressing immunoresponsive cells can elicit cytotoxic effect via sensitization of tumor cells to proinflammatory cytokines. In certain embodiments, the LIGHT polypeptide increases anti-tumor cytokine production of the immunoresponsive cell. Non-limiting examples of anti-tumor cytokines include IL-33, IL-5, IL-9, IL-13, IL-2, granulocyte macrophage colony-stimulating factor (GM-CSP), IFN-γ, TNF-super family, Fas ligand (Fas-L), TNF-α, perforin, granzyme B, and granzyme A.

Also, the co-stimulatory potential of LIGHT provide the immunoresponsive cells with enhanced activation signal, increased proliferation potential and persistence. This co-stimulatory effect may not only be specific to the immunoresponsive cells (e.g., CAR-T cell), but also to other endogenous immune cells, which can reactivate or induce an existing endogenous antitumor response.

Previous studies have shown that constitutively expressing LIGHT has a hyper-activated T cell population putting them at risk for spontaneous autoimmunity. However, engineered immunoresponsive cell therapy (e.g., CAR-T cell therapy) is a tumor-targeted delivery system, which minimizes systemic toxicity and effect while applying a controlled dosage of biologically active molecules to the tumor microenvironment. In addition, LIGHT can act in both autocrine and paracrine signaling to produce more proinflammatory cytokines in the tumor microenvironment, essentially increasing the tumor immunogenicity. Furthermore, the ability of LIGHT to normalize tumor vasculature and drive the formation of secondary and tertiary lymphoid structure (TLS) at tumor sites can drastically enhance the effect of tumor-infiltrating lymphocyte (TIL) infiltration and penetration. Overall, the presently disclosed LIGHT-overexpressing immunoresponsive cells can not only boost the effect of the immunoresponsive cells but also elicit an endogenous antitumor response through other immune cells.

The inventors discovered that the expression of LTβR or a portion thereof in a target cell can impact the anti-tumor activity of the presently disclosed LIGHT-overexpressing immunoresponsive cells toward the target cell. For example, it was observed that the presently disclosed LIGHT-overexpressing immunoresponsive cells had substantially reduced killing capacity toward a target cell whose LTβR is knocked out or a target cell comprising a truncated LTβR lacking a functional intracellular domain of LTβR.

A number of tumor cells express LTβR, including, but not limited to, blood cancer (including, but not limited to, acute myeloid leukemia (AML), chronic lymphocytic leukemia, B cell leukemia, multiple myeloma, lymphoblastic leukemia (ALL), Hodgkin's lymphoma, and non-Hodgkin's lymphoma), solid tumors (including, but not limited to, pancreatic cancer, ovarian cancer, lung cancer, melanoma, colon cancer, colorectal cancer, mesothelioma, osteosarcoma, cholangiocarcinoma, and breast cancer).

The presently disclosed LIGHT-overexpressing immunoresponsive cells confer anti-tumor activity and cytolytic activity toward cells expressing a medium expression level or a high expression level of the target antigen to which the antigen recognizing receptor binds. Surprisingly, the presently disclosed LIGHT-overexpressing immunoresponsive cells confer anti-tumor activity and cytolytic activity toward cells that have a low expression level of the target antigen to which the antigen recognizing receptor binds (“antigen-low cells”). Such anti-tumor activity and cytolytic activity exhibited by the presently disclosed LIGHT-overexpressing immunoresponsive cells toward antigen-low cells are significantly improved as compared to the corresponding immunoresponsive cells that do not overexpress LIGHT. In certain embodiments, a low expression level is less than about 1500 molecules per cell, less than about 1400 molecules per cell, less than about 1300 molecules per cell, less than about 1200 molecules per cell, less than about 1100 molecules per cell, less than about 1000 molecules per cell, or less than about 900 molecules per cell. In certain embodiments, a high expression level is about 2500 or greater molecules per cell, about 3000 or greater molecules per cell, about 4000 or greater molecules per cell, or about 5000 or greater molecules per cell. In certain embodiments, a medium expression level is between about 1500 and about 5000 molecules per cell, between about 1500 and about 4000 molecules per cell, between about 1500 and about 3000 molecules per cell, or between about 1500 and about 2500 molecules per cell.

Furthermore, surprisingly, the presently disclosed LIGHT-overexpressing immunoresponsive cells confer anti-tumor activity and cytolytic activity toward cells that do not express the target antigen to which the antigen recognizing receptor binds. In certain embodiments, the immunoresponsive cells comprise (a) an antigen-recognizing receptor (e.g., a CAR) that binds to an antigen, and (b) an exogenous LIGHT polypeptide. In certain embodiments, the immunoresponsive cells comprise (a) an antigen-recognizing receptor (e.g., a CAR) that binds to an antigen and (b) a nucleic acid encoding a LIGHT polypeptide.

In certain embodiments, the antigen-recognizing receptor is capable of activating the immunoresponsive cell. In certain embodiments, the LIGHT polypeptide is capable of promoting an anti-tumor effect of the immunoresponsive cell. The immunoresponsive cells can be transduced with an antigen-recognizing receptor and an exogenous LIGHT polypeptide such that the cells co-express the antigen-recognizing receptor and the exogenous LIGHT polypeptide.

In certain embodiments, the cell is selected from the group consisting of cells of lymphoid lineage and cells of myeloid lineage. In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, and stem cells from which lymphoid cells may be differentiated. In certain embodiments, the stem cell is a pluripotent stem cell. In certain embodiments, the pluripotent stem cell is an embryonic stem cell or an induced pluripotent stem cell (iPSC).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, regulatory T cells (also known as suppressor T cells), tumor-infiltrating lymphocyte (TIL), Natural Killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the cell is a NK cell. Natural killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes, e.g., those disclosed in Sadelain et al., Nat Rev Cancer (2003); 3:35-45 (disclosing peripheral donor lymphocytes genetically modified to express CARs), in Morgan, R. A., etal. 2006 Science 314:126-129 (disclosing peripheral donor lymphocytes genetically modified to express a full-length tumor antigen recognizing T cell receptor complex comprising the a and β heterodimer), in Panelli et al., J Immunol (2000); 164:495-504; Panelli et al., J Immunol (2000); 164:4382-4392 (disclosing lymphocyte cultures derived from tumor infiltrating lymphocytes (TILs) in tumor biopsies), and in Dupont et al., Cancer Res (2005); 65:5417-5427; Papanicolaou et al., Blood (2003); 102:2498-2505 (disclosing selectively in vitro-QxpandQd antigen specific peripheral blood leukocytes employing artificial antigen-presenting cells (AAPCs) or pulsed dendritic cells).

The cells (e.g., T cells) can be autologous, non-autologous (e.g., allogeneic), or derived in vitro from engineered progenitor or stem cells.

The cells of the presently disclosed subject matter can be cells of the myeloid lineage. Non-limiting examples of cells of the myeloid lineage include monocytes, macrophages, neutrophils, dendritic cells, basophils, neutrophils, eosinophils, megakaryocytes, mast cell, erythrocyte, thrombocytes, and stem cells from which myeloid cells may be differentiated. In certain embodiments, the stem cell is a pluripotent stem cell. In certain embodiments, the pluripotent stem cell is an embryonic stem cell or an induced pluripotent stem cell (iPSC).

In certain embodiments, the presently disclosed cells are capable of modulating the tumor microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4+ T cells (Tregs), myeloid derived suppressor cells (MDSCs), tumor associated macrophages (TAMs), immune suppressive cytokines including TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective antitumor immune response. Collectively these immune suppressive factors can induce either marked anergy or apoptosis of adoptively transferred CAR modified T cells upon encounter with targeted tumor cells.

In certain embodiments, the cell further comprises a soluble single-chain variable fragment (scFv) that binds to a polypeptide that has immunosuppressive activity or immunostimulatory activity. In certain embodiments, immunosuppressive activity refers to induction of signal transduction or changes in protein expression in a cell (e.g., an activated immunoresponsive cell) resulting in a decrease in an immune response. Polypeptides known to suppress or decrease an immune response via their binding include CD47, PD-1, CTLA-4, and their corresponding ligands, including SIRPa, PD-L1, PD-L2, B7-1, and B7-2. Such polypeptides are present in the tumor microenvironment and inhibit immune responses to neoplastic cells. In certain embodiments, inhibiting, blocking, or antagonizing the interaction of immunosuppressive polypeptides and/or their ligands enhances the immune response of the immunoresponsive cell.

In certain embodiments, immunostimulatory activity refers to induction of signal transduction or changes in protein expression in a cell (e.g., an activated immunoresponsive cell) resulting in an increase in an immune response. Immunostimulatory activity may include pro-inflammatory activity. Polypeptides known to stimulate or increase an immune response via their binding include CD28, OX40, 4-1BB, and their corresponding ligands, including B7-1, B7-2, OX-40L, and 4-1BBL. Such polypeptides are present in the tumor microenvironment and activate immune responses to neoplastic cells. In certain embodiments, promoting, stimulating, or agonizing pro-inflammatory polypeptides and/or their ligands enhances the immune response of the immunoresponsive cell.

Cells comprising an antigen-recognizing receptor (e.g., a CAR) and a soluble scFv that binds to a polypeptide that has immunosuppressive activity or immunostimulatory activity are disclosed in International Patent Publication No. WO 2014/134165, which is incorporated by reference in its entirety.

In certain embodiments, the presently disclosed cell further comprises an exogenous CD40L. Cells comprising an antigen-recognizing receptor (e.g., a CAR) and an exogenous CD40L are disclosed in International Patent Publication No. WO 2014/134165, which is incorporated by reference in its entirety.

Furthermore, in certain embodiments, the presently disclosed cell is engineered to express IL-18. In certain embodiments, the cell further comprises an exogenous IL-18 polypeptide. In certain embodiments, the exogenous IL-18 polypeptide comprises or consists of the amino acid sequence set forth in SEQ ID NO: 52 or a fragment thereof. SEQ ID NO: 52 is provided below.

[SEQ ID NO: 52]
MGYRMQLLSCIALSLALVTNSGYFGKLESKLSVIRNLNDQVLFIDQGNRP
LFEDMTDSDCRDNAPRTIFIISMYKDSQPRGMAVTISVKCEKISTLSCEN
KIISFKEMNPPDNIKDTKSDIIFFQRSVPGHDNKMQFESSSYEGYFLACE
KERDLFKLILKKEDELGDRSIMFTVQNED

Cells comprising an antigen-recognizing receptor (e.g., a CAR) and engineered to express IL-18 are disclosed in International Patent Publication No. WO2018/027155, which is incorporated by reference in its entirety.

Additionally or alternatively, the presently disclosed cell is engineered to express IL-33. In certain embodiments, the cell further comprises an exogenous IL-33 polypeptide. In certain embodiments, the exogenous IL-33 polypeptide comprises or consists of the amino acid sequence set forth in SEQ ID NO: 53 or a fragment thereof. SEQ ID NO: 53 is provided below.

[SEQ ID NO: 53]
MYRMQLLSCIALSLALVINSSITGISPITEYLASLSTYNDQSITFALEDE
SYEIYVEDLKKDEKKDKVLLSYYESQHPSNESGDGVDGKMLMVTLSPTKD
FWLHANNKEHSVELHKCEKPLPDQAFFVLHNMHSNCVSFECKTDPGVFIG
VKDNHLALIKVDSSENLCTENILFKLSET

Cells comprising an antigen-recognizing receptor (e.g., a CAR) and engineered to express IL-33 are disclosed in International Patent Publication No. WO2019/099479, which is incorporated by reference in its entirety.

Additionally or alternatively, the presently disclosed cell is engineered to express IL-36. In certain embodiments, the cell further comprises an exogenous IL-36 polypeptide. Cells comprising an antigen-recognizing receptor (e.g., a CAR) and engineered to express IL-36 are disclosed in International Patent Publication No. WO2019/099483, which is incorporated by reference in its entirety.

In certain embodiments, the presently disclosed immunoresponsive cells are capable of activating endogenous immune cells. In certain embodiments, the endogenous immune cells are selected from the group consisting of NK cells, NK-T cells, dendritic cells and endogenous CD8 T cells. In certain embodiments, the immunoresponsive cells disclosed herein increase the endogenous immune cells population. In certain embodiments, the immunoresponsive cells comprising an antigen recognizing receptor and a LIGHT polypeptide (e.g., an exogenous LIGHT polypeptide) increase the endogenous immune cells population by at least about 5%, about 10%, about 20%, about 30%, about 40%, about 50%, about 60%, about 70%, about 80%, about 90%, about 100%, about 150%, about 200%, about 250%, about 300%, about 400%, about 500%, about 600%, about 700%, about 800%, about 900%, about 1000%, or more compared to immunoresponsive cells comprising an antigen recognizing receptor alone (e.g., not comprising a—LIGHT polypeptide)

### 5.5 Nucleic Acid Compositions and Vectors

The presently disclosed subject matter provides nucleic acid compositions comprising a first nucleic acid encoding an antigen-recognizing receptor (e.g., one disclosed in Section 5.3) and a second nucleic acid encoding a LIGHT polypeptide (e.g., one disclosed in Section 5.2). Also provided are cells comprising such nucleic acid compositions.

In certain embodiments, the first nucleic acid is operably linked to a first promoter. In certain embodiments, the second nucleic acid is operably linked to a second promoter. In certain embodiments, the first promoter is the same as the second promoter, i.e., the expression of the antigen recognizing receptor and the expression of the LIGHT polypeptide are under the control of the same promoter.

In certain embodiments, the promoter is endogenous or exogenous. In certain embodiments, the exogenous promoter is selected from the group consisting of an elongation factor (EF)-1 promoter, a cytomegalovirus immediate-early promoter (CMV) promoter, a simian virus 40 early promoter (SV40) promoter, a phosphoglycerate kinase (PGK) promoter, a metallothionein promoter, ubiquitin C (Ubc) promoter, a β-actin promoter, and a CAG promoter. The promoter can be a constitutive promoter or an inducible promoter.

In certain embodiments, the promoter is a constitutive promoter.

In certain embodiments, the promoter is a constitutive promoter. In certain embodiments, the LIGHT polypeptide is constitutively expressed. In certain embodiment, the constitutive promoter is selected from the group consisting of a retroviral long terminal repeat (LTR), a CMV promoter, an EF1α promoter, a SV40 promoter, a PGK1 promoter, a Ubc promoter, a β-actin promoter, a CAG promoter, a NFAT transcriptional response element (TRE) promoter, a CD69 promoter, a CD25 promoter, an IL-2 promoter, and an estrogen response element (ERE) promoter. Non-limiting examples of inducible promoters include a CMV promoter, an EF1α promoter, a SV40 promoter, a PGK1 promoter, a Ubc promoter, a β-actin promoter, a CAG promoter, a NFAT transcriptional response element (TRE) promoter, a CD69 promoter, a CD25 promoter, an IL-2 promoter, and an estrogen response element (ERE) promoter.

The nucleic acid compositions can be administered to subjects or and/delivered into cells by art-known methods or as described herein. Genetic modification of a cell (e.g., a T cell or a NK cell) can be accomplished by transducing a substantially homogeneous cell composition with a recombinant DNA construct. In certain embodiments, a retroviral vector (e.g., gamma-retroviral vector or lentiviral vector) is employed for the introduction of the DNA construct into the cell. For example, a polynucleotide encoding an antigen-recognizing receptor can be cloned into a retroviral vector and expression can be driven from its endogenous promoter, from the retroviral long terminal repeat, or from a promoter specific for a target cell type of interest. Non-viral vectors may be used as well.

For initial genetic modification of a cell to include a presently disclosed antigen-recognizing receptor (e.g., a CAR or a TCR), a retroviral vector can be employed for transduction, however any other suitable viral vector or non-viral delivery system can be used. The antigen-recognizing receptor and LIGHT polypeptide can be constructed in a single, multi cis-tronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picomavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller et al., (1985) Mol Cell Biol (1985); 5:431-437); PA317 (Miller., etal.,Mol Cell Biol (1986); 6:2895-2902); and CRIP (Danos et al., Proc Natl Acad Sci USA (1988); 85:6460-6464). Non-amphotropic particles are suitable too, e.g., particles pseudotyped with VSVG, RD114 or GALV envelope and any other known in the art.

Possible methods of transduction also include direct co-culture of the cells with producer cells (Bregni et al., Blood (1992); 80:1418-1422), or culturing with viral supernatant alone or concentrated vector stocks with or without appropriate growth factors and polycations (Xu etal., Exp Hemat (1994); 22:223-230; and Hughes etal. J Clin Invest (1992); 89:1817).

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Thera (1990); 15-14; Friedman, Science 244:1275-1281, 1989; Eglitis et al., BioTechniques (1988); 6:608-614; Tolstoshev et al., Cur Opin Biotechnol (1990); 1:55-61; Sharp, The Lancet (1991); 337:1277-78; Cometta et al., Nucleic Acid Research and Molecular Biology 36:311-22, 1987; Anderson, Science (1984); 226:401-409; Moen, Blood Cells 17:407-16, 1991; Miller et al., Biotechnol (1989); 7:980-90; LeGal La Salle et al., Science (1993); 259:988-90; and Johnson, Chest (1995)107: 77S-83S). Retroviral vectors are particularly well developed and have been used in clinical settings (Rosenberg et al., N Engl J Med (1990); 323:370, 1990; Anderson et al., U.S. Pat. No. 5,399,346).

Non-viral approaches can also be employed for genetic modification of a cell. For example, a nucleic acid molecule can be introduced into a cell by administering the nucleic acid in the presence of lipofection (Feigner et al., Proc Natl Acad Sci U.S.A. (1987); 84:7413; Ono et al., Neurosci Lett (1990); 17:259; Brigham et al., Am J Med Sci (1989); 298:278; Staubinger et al., Methods inEnzymol (1983); 101:512, Wu et al., J Biol Chern (1988); 263:14621; Wu etal., J Biol Chern (1989); 264:16985), or by microinjection under surgical conditions (Wolff et al., Science (1990); 247:1465). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Any targeted genome editing methods can also be used to deliver a presently disclosed antigen-recognizing receptor to a cell or a subject. In certain embodiments, a CRISPR system is used to deliver a presently disclosed antigen-recognizing receptor disclosed herein. In certain embodiments, zinc-finger nucleases are used to deliver the antigen-recognizing receptor. In certain embodiments, a TALEN system is used to deliver a presently disclosed antigen-recognizing receptor.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type Ils restriction endonuclease Fokl. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome. Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

### 5.6 Polypeptides

The presently disclosed subject matter provides methods for optimizing an amino acid sequence or a nucleic acid sequence by producing an alteration in the sequence. Such alterations may include certain mutations, deletions, insertions, or post-translational modifications. The presently disclosed subject matter further includes analogs of any naturally-occurring polypeptides disclosed herein (including, but not limited to, LIGHT, CD371, CD8, CD28, 4-1BB, CD32, IL-18, IL-33). Analogs can differ from a naturally-occurring polypeptide disclosed herein by amino acid sequence differences, by post-translational modifications, or by both. Analogs can exhibit at least about 85%, about 90%, about 91%, about 92%, about 93%, about 94%, about 95%, about 96%, about 97%, about 98%, about 99% or more homologous or identical to all or part of a naturally-occurring amino, acid sequence of the presently disclosed subject matter. The length of sequence comparison is at least 5, 10, 15 or 20 amino acid residues, e.g., at least 25, 50, or 75 amino acid residues, or more than 100 amino acid residues. Again, in an exemplary approach to determining the degree of identity, a BLAST program may be used, with a probability score between e⁻³ and e⁻¹⁰⁰ indicating a closely related sequence.

Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

In addition to full-length polypeptides, the presently disclosed subject matter also provides fragments of any of the polypeptides disclosed herein. As used herein, the term “a fragment” means at least about 5, about 10, or about 15 amino acids. In certain embodiments, a fragment comprises at least 20 contiguous amino acids, at least about 30 contiguous amino acids, at least about 40 contiguous amino acids, or at least about 50 contiguous amino acids. Fragments can be generated by methods known to those skilled in the art or may result from normal protein processing (e.g., removal of amino acids from the nascent polypeptide that are not required for biological activity or removal of amino acids by alternative mRNA splicing or alternative protein processing events).

### 5.7 Formulations and Administration

The presently disclosed subject matter also provides compositions comprising the presently disclosed cells. In certain embodiments, the composition is a pharmaceutical composition comprising the presently disclosed cells and a pharmaceutically acceptable carrier.

Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified cells.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Compositions comprising the presently disclosed cells can be provided systemically or directly to a subject for inducing and/or enhancing an immune response to an antigen and/or treating and/or preventing a neoplasia. In certain embodiments, the presently disclosed cells or compositions comprising thereof are directly injected into an organ of interest (e.g., an organ affected by a neoplasia). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of cells (e.g., T cells or NK cells) in vitro or in vivo.

The presently disclosed cells can be administered in any physiologically acceptable vehicle, normally intravascularly, although they may also be introduced into bone or other convenient site where the cells may find an appropriate site for regeneration and differentiation (e.g., thymus).

The quantity of cells to be administered can vary for the subject being treated. In certain embodiments, between about 10⁴ and about 10¹⁰, between about 10⁴ and about 10⁷, between about 10⁵ and about 10⁷, between about 10⁵ and about 10⁹, or between about 10⁶ and about 10⁸ of the presently disclosed cells are administered to a subject. More effective cells may be administered in even smaller numbers. Usually, at least about 1×10⁵ cells will be administered, eventually reaching about 1×10¹⁰ or more. In certain embodiments, at least about 1×10⁵, 5×10⁵, 1×10⁶, about 5×10⁶, about 1×10⁷, about 5×10⁷, about 1×10⁸, or about 5×10⁸ of the presently disclosed cells are administered to a subject. In certain embodiments, between about 1×10⁵ and 1×10⁷ of the presently disclosed cells are administered to a subject. In certain embodiments, between about 1×10⁵ and 5×10⁶ of the presently disclosed cells are administered to a subject. In certain embodiments, between about 5×10⁵ and 2×10⁶ of the presently disclosed cells are administered to a subject. In certain embodiments, about 1×10⁶ of the presently disclosed cells are administered to a subject. In certain embodiments, about 2×10⁶ of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The presently disclosed cells can comprise a purified population of cells. Those skilled in the art can readily determine the percentage of the presently disclosed cells in a population using various well-known methods, such as fluorescence activated cell sorting (FACS). Suitable ranges of purity in populations comprising the presently disclosed immunoresponsive cells are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like.

Administration of the compositions can be autologous or heterologous. For example, cells can be obtained from one subject, and administered to the same subject or a different, compatible subject. Peripheral blood derived cells or their progeny (e.g., in vivo, ex vivo or in vitro derived) can be administered. When administering a presently disclosed composition (e.g., a pharmaceutical composition comprising presently disclosed cells), it can be formulated in a unit dosage injectable form (solution, suspension, emulsion).

The presently disclosed cells and compositions can be administered by any method known in the art including, but not limited to, oral administration, intravenous administration, subcutaneous administration, intranodal administration, intratumoral administration, intrathecal administration, intravascular administration (e.g., retro-orbital injection), intrapleural administration, intraosseous administration, intraperitoneal administration, pleural administration, and direct administration to the subject. In certain embodiments, the presently disclosed cells or compositions are administered to a subject intravenously. The presently disclosed immunoresponsive cells can be administered in any physiologically acceptable vehicle, normally intravascularly, although they may also be introduced into bone or other convenient site where the cells may find an appropriate site for regeneration and differentiation (e.g., thymus).

Compositions comprising the presently disclosed immunoresponsive cells can be provided systemically or directly to a subject for inducing and/or enhancing an immune response to an antigen, inhibiting growth of a LTβR-expressing cell, and/or treating a tumor, pathogen infection, or infectious disease. In certain embodiments, the presently disclosed immunoresponsive cells or compositions comprising thereof are directly injected into an organ of interest (e.g., an organ affected by a neoplasm). Alternatively, the presently disclosed immunoresponsive cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of T cells, NK cells, or CTL cells in vitro or in vivo.

### 5.8 Methods of Treatment

The presently disclosed cells and compositions comprising thereof can be used in a therapy or medicament. The presently disclosed subject matter provides various methods of using the cells (e.g., T cells) or compositions comprising thereof. For example, the presently disclosed cells and compositions comprising thereof can be used for inhibiting growth of a cell that expresses lymphotoxin-β receptor (LTβR) or a portion thereof. The method comprises contacting a cell that expresses LTβR or a portion thereof with the immunoresponsive cell disclosed herein or the composition disclosed herein.

The inventors discovered that the expression of a functional LTβR in a target cell can impact the anti-tumor activity of the presently disclosed LIGHT-overexpressing immunoresponsive cells toward the target cell. For example, it was observed that the presently disclosed LIGHT-overexpressing immunoresponsive cells had substantially reduced killing capacity toward a target cell whose LTβR is knocked out or a target cell comprising a truncated LTβR lacking the intracellular domain of LTβR.

LTβR is also known as Tumor necrosis factor receptor superfamily member 3 (TNR3). In certain embodiments, the LTβR is a human LTβR. In certain embodiments, the LTβR is a wild-type human LTβR. In certain embodiments, the wild-type human LTβR comprises the amino acid sequence with a Uniprot Reference No: P36941-1 (SEQ ID NO: 54). SEQ ID NO: 54 is provided below.

[SEQ ID NO: 54]
MLLPWATSAPGLAWGPLVLGLFGLLAASQPQAVPPYASENQTCRDQEKEY
YEPQHRICCSRCPPGTYVSAKCSRIRDTVCATCAENSYNEHWNYLTICQL
CRPCDPVMGLEEIAPCTSKRKTQCRCQPGMFCAAWALECTHCELLSDCPP
GTEAELKDEVGKGNNHCVPCKAGHFQNTSSPSARCQPHTRCENQGLVEAA
PGTAQSDTTCKNPLEPLPPEMSGTMLMLAVLLPLAFFLLLATVFSCIWKS
HPSLCRKLGSLLKRRPQGEGPNPVAGSWEPPKAHPYFPDLVQPLLPISGD
VSPVSTGLPAAPVLEAGVPQQQSPLDLTREPQLEPGEQSQVAHGTNGIHV
TGGSMTITGNIYIYNGPVLGGPPGPGDLPATPEPPYPIPEEGDPGPPGLS
TPHQEDGKAWHLAETEHCGATPSNRGPRNQFITHD

In certain embodiments, a human LTβR comprises an extracellular domain, a transmembrane domain, and a cytoplasmic domain. In certain embodiments, the extracellular domain comprises amino acids 31 to 227 of SEQ ID NO: 54. In certain embodiments, the transmembrane domain comprises amino acids 228 to 248 of SEQ ID NO: 54. In certain embodiments, the cytoplasmic domain comprises amino acids 249 to 435 of SEQ ID NO: 54.

In certain embodiments, the target cell expresses a functional LTβR. In certain embodiments, the functional LTβR comprises at least a portion of the intracellular domain of LTβR. In certain embodiments, the functional LTβR comprises amino acids 249 to 435 of SEQ ID NO: 54 or a consecutive portion thereof, which is at least about 10, at least about 15, at least about 20, at least about 25, at least about 30, at least about 40, at least about 50, at least about 60, at least about 70, at least about 80, at least about 90, or at least about 100, and up to 187 amino acids in length. In certain embodiments, the functional LTβR comprises amino acids 249 to 435 of SEQ ID NO: 54.

In addition, the presently disclosed cells and compositions comprising thereof can be used for reducing tumor burden in a subject. The presently disclosed cell can reduce the number of tumor cells, reduce tumor size, and/or eradicate the tumor in the subject. The presently disclosed cells and compositions comprising thereof can be used for treating a tumor in a subject. The presently disclosed cells and compositions comprising thereof can be used for prolonging the survival of a subject suffering from a tumor. The presently disclosed cells and compositions comprising thereof can be used for treating a pathogen infection or an infectious disease.

Such methods comprise administering the presently disclosed cells or a composition (e.g., a pharmaceutical composition) comprising thereof to achieve the desired effect, e.g., palliation of an existing condition or prevention of recurrence. For treatment, the amount administered is an amount effective in producing the desired effect. An effective amount can be provided in one or a series of administrations. An effective amount can be provided in a bolus or by continuous perfusion.

The presently disclosed subject matter provides various methods of using the cells (e.g., T cells) or compositions comprising thereof. For example, the presently disclosed subject matter provides methods of inhibiting growth of a LTβR-expressing cell. In certain embodiments, the method of inhibiting growth of a LTβR-expressing cell comprises contacting a LTβR-expressing cell with the presently disclosed cells or a composition comprising thereof.

For example, the presently disclosed subject matter provides methods of reducing tumor burden in a subject. In certain embodiments, the method of reducing tumor burden comprises administering the presently disclosed cells or a composition comprising thereof to the subject. The presently disclosed cell can reduce the number of tumor cells, reduce tumor size, and/or eradicate the tumor in the subject.

The presently disclosed subject matter also provides methods of increasing or lengthening survival of a subject having a tumor. In certain embodiments, the method of increasing or lengthening survival of a subject having a tumor comprises administering the presently disclosed immunoresponsive cells or a composition comprising thereof to the subject. The method can reduce or eradicate tumor burden in the subject.

Non-limiting examples of tumors include acute myeloid leukemia (AML), multiple myeloma, Chronic Lymphocytic Leukemia (CLL), B cell leukemia, multiple myeloma, lymphoblastic leukemia (ALL), lymphoma (Hodgkin's lymphoma, non-Hodgkin's lymphoma), glioblastoma, myelodysplastic syndrome (MDS), and chronic myelogenous leukemia (CML), bone cancer, intestinal cancer, liver cancer, skin cancer, cancer of the head or neck, melanoma (cutaneous or intraocular malignant melanoma), renal cancer (e.g. clear cell carcinoma), throat cancer, prostate cancer (e.g. hormone refractory prostate adenocarcinoma), blood cancers (e.g. leukemias, lymphomas, and myelomas), uterine cancer, rectal cancer, cancer of the anal region, bladder cancer, brain cancer, stomach cancer, testicular cancer, carcinoma of the fallopian tubes, carcinoma of the endometrium, carcinoma of the cervix, carcinoma of the vagina, carcinoma of the vulva, leukemias (e.g., acute leukemia, acute lymphocytic leukemia, acute myelocytic leukemia, acute myeloblastic leukemia, acute promyelocytic leukemia, acute monocytic leukemia, acute erythroleukemia, chronic leukemia, chronic myelocytic leukemia, polycythemia vera, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, include Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, nile duct carcinoma, choriocarcinoma, seminoma, embryonal carcinoma, Wilm's tumor, cervical cancer, salivary gland cancer, uterine cancer, testicular cancer, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

In certain embodiments, a cell of the tumor expresses LTβR or a portion thereof. Non-limiting tumors having a cell expressing LTβR or a portion thereof include pancreatic cancer, ovarian cancer, lung cancer, melanoma, colon cancer, colorectal cancer, mesothelioma, osteosarcoma, cholangiocarcinoma, breast cancer, and acute myeloid leukemia (AML).

In certain embodiments, the tumor is blood cancer. Non-limiting examples of blood cancer include acute myeloid leukemia (AML), chronic lymphocytic leukemia, B cell leukemia, multiple myeloma, lymphoblastic leukemia (ALL), and non-Hodgkin's lymphoma. In certain embodiments, the tumor is AML.

In certain embodiments, the tumor is solid tumor. Non-limiting examples of solid tumors include mesothelioma, lung cancer, pancreatic cancer, ovarian cancer, breast cancer, colon cancer, colorectal cancer, pleural tumor, glioblastoma, esophageal cancer, gastric cancer, synovial sarcoma, thymic carcinoma, endometrial carcinoma, stomach cancer, and cholangiocarcinoma. In certain embodiments, the solid tumor is selected from the group consisting of ovarian cancer, lung cancer, melanoma, colon cancer, colorectal cancer, mesothelioma, osteosarcoma, cholangiocarcinoma, and pancreatic cancer.

Additionally, the presently disclosed subject matter provides methods for increasing an immune response in a subject. In certain embodiments, the method of increasing an immune response in a subject comprises administering to the subject the presently disclosed cell or a composition comprising thereof.

Furthermore, the presently disclosed subject matter provides methods for treating a pathogen infection or an infectious disease. In certain embodiments, the method of treating a pathogen infection or an infectious disease comprises administering to a subject suffering from a pathogen infection or infectious disease the presently disclosed cell or a composition comprising thereof.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

As a consequence of surface expression of an antigen recognizing receptor, adoptively transferred cells (e.g., immunoresponsive cells, e.g., T cells or NK cells) are endowed with augmented and selective cytolytic activity at the tumor site. Furthermore, subsequent to their localization to tumor or viral infection and their proliferation, the cells turn the tumor or viral infection site into a highly conductive environment for a wide range of immune cells involved in the physiological anti-tumor or antiviral response (tumor infiltrating lymphocytes, NK-, NKT-cells, dendritic cells, and macrophages).

Further modification can be introduced to the presently disclosed cells (e.g., T cells) to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g, cetuximab). EGFRt can be covalently joined to the upstream of the antigen-recognizing receptor (e.g., CAR). The suicide gene can be included within the vector comprising nucleic acids encoding a presently disclosed antigen recognizing receptor (e.g., CAR). In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated cells expressing the antigenrecognizing receptor (e.g., CAR). The incorporation of a suicide gene into the a presently disclosed antigen-recognizing receptor (e.g., CAR) gives an added level of safety with the ability to eliminate the majority of receptor-expressing cells within a very short time period. A presently disclosed cell (e.g., a T cell) incorporated with a suicide gene can be pre-emptively eliminated at a given timepoint post the cell infusion, or eradicated at the earliest signs of toxicity.

### 5.9 Kits

The presently disclosed subject matter provides kits for inhibiting growth of a cell that expresses lymphotoxin-β receptor (LTβR) or a portion thereof, inducing and/or enhancing an immune response in a subject, treating a tumor in a subject, reducing tumor burden in a subject, increasing or lengthening survival of a subject having a tumor in a subject, and/or treating a pathogen infection or infectious disease. In certain embodiments, the kit comprises the presently disclosed cells or a composition comprising thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, the cells are provided together with instructions for administering the cells to a subject having a tumor, a pathogen infection or an infectious disease. The instructions generally include information about the use of the composition for the treatment of a tumor, a pathogen infection or an infectious disease. In certain embodiments, the instructions include at least one of the following: description of the therapeutic agent; dosage schedule and administration for treatment or prevention of a tumor or neoplasm; precautions; warnings; indications; counterindications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 6. EXAMPLES

The practice of the presently disclosed subject matter employs, unless otherwise indicated, conventional techniques of molecular biology (including recombinant techniques), microbiology, cell biology, biochemistry and immunology, which are well within the purview of the skilled artisan.

The following examples are put forth so as to provide those of ordinary skill in the art with a complete disclosure and description of how to make and use the presently disclosed cells and compositions, and are not intended to limit the scope of what the inventors regard as their invention.

### Example 1—Generation of LIGHT-Modified CAR T Cells

To investigate the potential of LIGHT to augment CAR T cell functions, healthy human donor T cells were engineered by a retroviral vector (RV) to express both CAR and LIGHT. An anti-CD371 small chain variable fragment (scFv, B10H4L) was fused to a myc tag for CAR detection, then human CD28 transmembrane and intracellular domains, and CD3% to generate a human second-generation CAR (B10HL28z). The B10H4L scFv comprises a CDR1, a CDR2, and a CDR3 of a V_{H} sequence set forth in SEQ ID NO: 10, and a CDR1, a CDR2, and a CDR3 of a V_{L} sequence set forth in SEQ ID NO: 11. The V_{H} and V_{L} are linked by a linker consisting of the amino acid sequence set forth in SEQ ID NO: 1. The V_{H} and V_{L} are positioned from the N- to the C-terminus: V_{H}-V_{L}. The B10H4L scFv comprises the amino acid sequence set forth in SEQ ID NO: 18. A self-cleaving element, P2A, was inserted after CD32 before the addition of the human LIGHT transgene allowing for the expression of both CAR and LIGHT from the same retroviral vector backbone (B10HL28z-LIGHT).

To further examine the functions of the LIGHT-modified CAR T cell in solid tumors, CAR T cell constructs utilizing an anti-mesothelin scFv that targeted the tumor-associated antigen, mesothelin, were generated. The anti-mesothelin scFv comprises a CDR1, a CDR2, and a CDR3 of a V_{H} sequence set forth in SEQ ID NO: 30, and a CDR1, a CDR2, and a CDR3 of a V_{L} sequence set forth in SEQ ID NO: 31. The anti-mesothelin scFv was fused to a myc tag for CAR detection, then human CD28 transmembrane and intracellular domains, and CD3ζ to generate a human second-generation CAR (mesothelin_28z). A self-cleaving element, P2A, was inserted after CD3 before the addition of the human LIGHT transgene allowing for the expression of both CAR and LIGHT from the same retroviral vector backbone (mesothelin_28z_LIGHT).

To investigate the potential of modified LIGHT to ameliorate proliferation defects, a truncated (Gly-LIGHT) version of LIGHT and a non-cleave (NC-LIGHT) version of LIGHT were generated by modifying specific residues that prevented proteolytic cleavage by proteases. Gly-LIGHT consists of the amino acid sequence set forth in SEQ ID NO: 8. The NC-LIGHT consists of the amino acid sequence set forth in SEQ ID NO: 9.

### Results

The following CAR T cell constructs were manufactured: “CD371 DEL”, “CD371_28z” or “B10HL28z”, “CD371 28z LIGHT” or “B10HL28z-LIGHT”, “B10HL28z-GlyLIGHT”, “B10HL28z-NCLIGHT”, “Meso_Del”, “Meso_Del-LT”, “Meso_28z”, “Meso_28z LIGHT”, “Meso_28z_GlyLIGHT”, and “Meso_28z_NCLIGHT”. The structures of the CARs are illustrated in FIG. 3A and FIG. 3B.

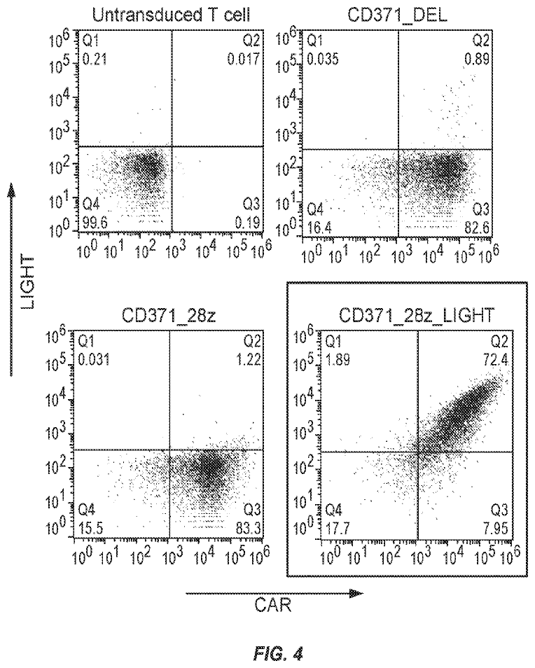

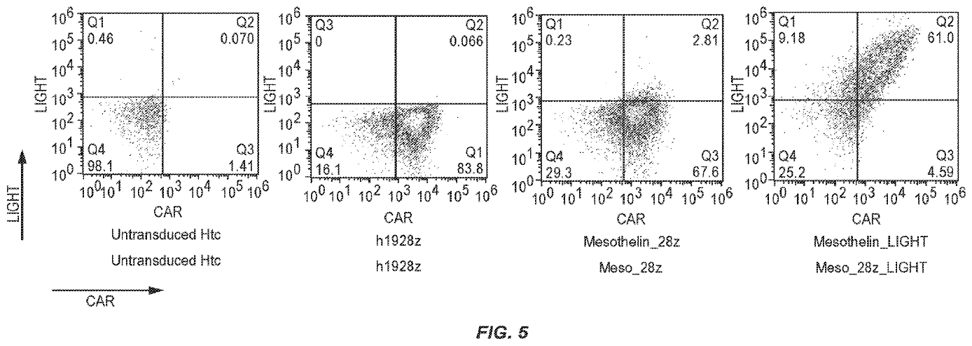

Transgene expression after retroviral transduction of human T cells was analyzed by flow cytometry and the results are shown in FIG. 4 and FIG. 5. Antibodies against myc-tag and LIGHT detected surface expression of CAR and LIGHT in the CAR T cell constructs. Controls included un-transduced T cell, CD371-targeted DEL CAR T cell which lacked the intracellular signaling domain, and anti-CD19 CAR “1928z”. As shown in FIG. 4 and FIG. 5, CD371-targeted CAR, mesothelin-targeted CAR, and LIGHT were expressed on the surface of transduced human T cells.

### Example 2—In Vitro Cytotoxicity Activities of CD371-Targeted CAT T Cells

To evaluate the efficacy of LIGHT-modified CAR T cells to target human myeloid leukemia (AML) cancer cells with different CD371 expressing level, human T cells were transfected with the CD371-targeted CARs (e.g., “B10H4L28z” and “B10H4L28z-LIGHT”).

The following cancer cell lines were used: U937 (CD371-expressing AML cell line), U937 CD371 KO (CD371-knockout AML cell line), OCI-AML3 (AML cell line expressing low or no CD371), SET2 (AML cell line expressing low or no CD371), and three solid tumor cell lines that did not express CD371, i.e., A375 (melanoma cell line), HT29 (colorectal adenocarcinoma cell line), and 8988t (pancreatic adenocarcinoma cell line). These cell lines were transduced with GFP-firefly luciferase for tumor tracking. Plots were representative of three independent experiments. Data errors were analyzed with mean±SEM.

### Results

### LIGHT-Modified CAR T Cells Prevented Outgrowth of Antigen-Negative Tumor Cells

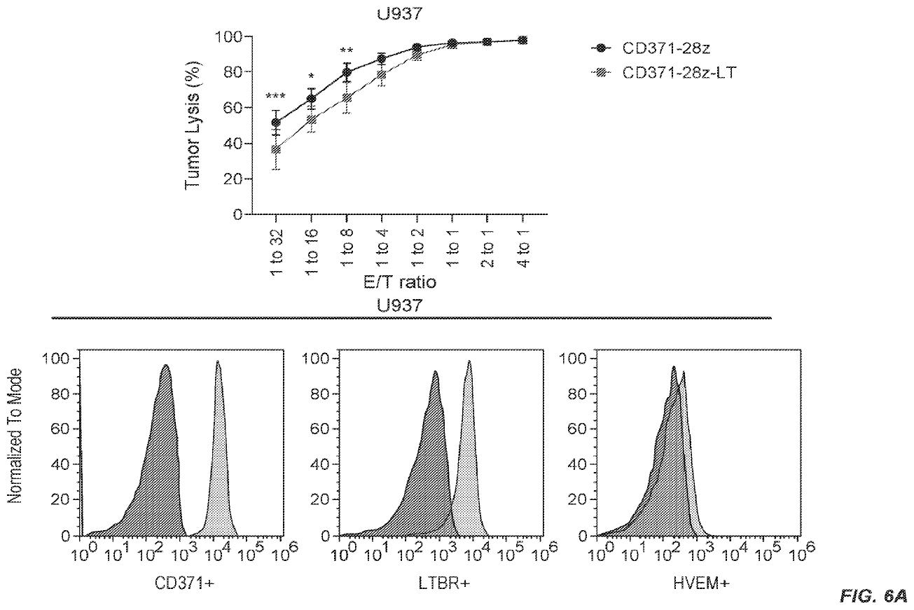

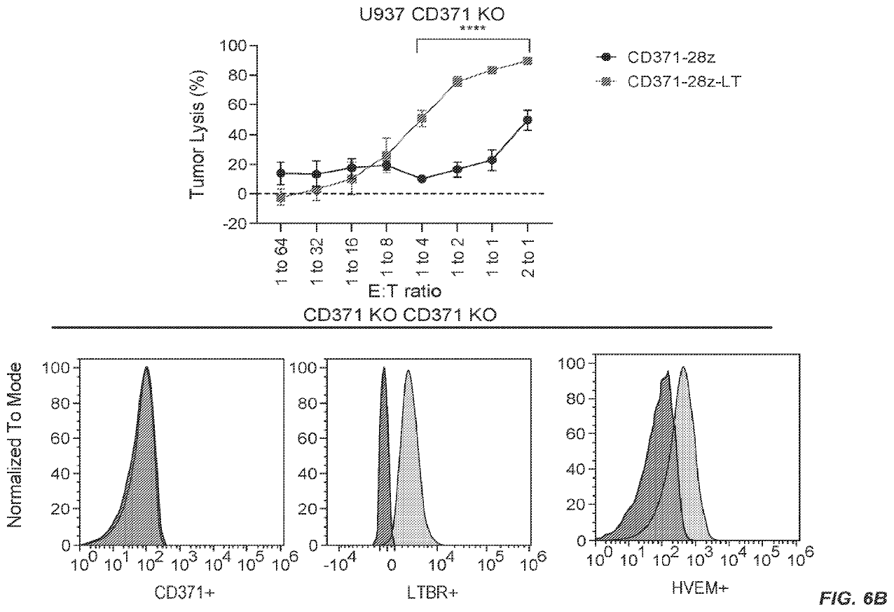

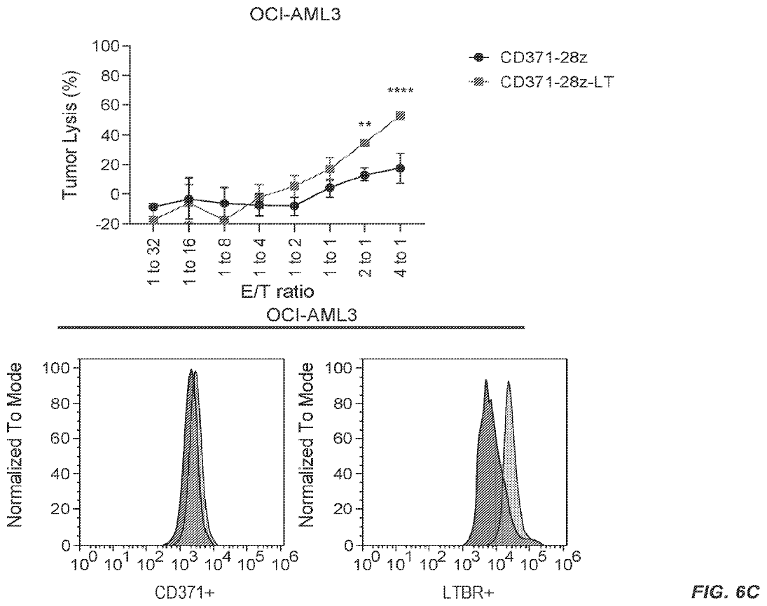

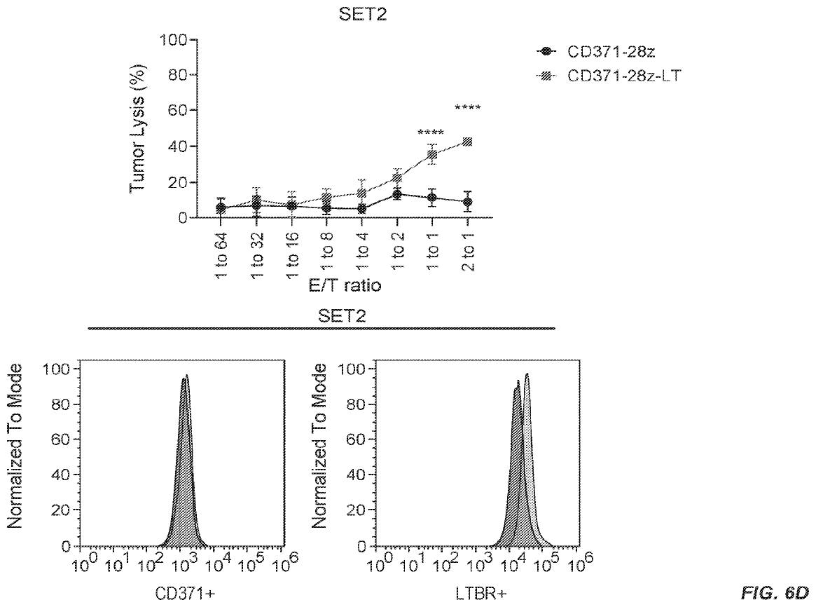

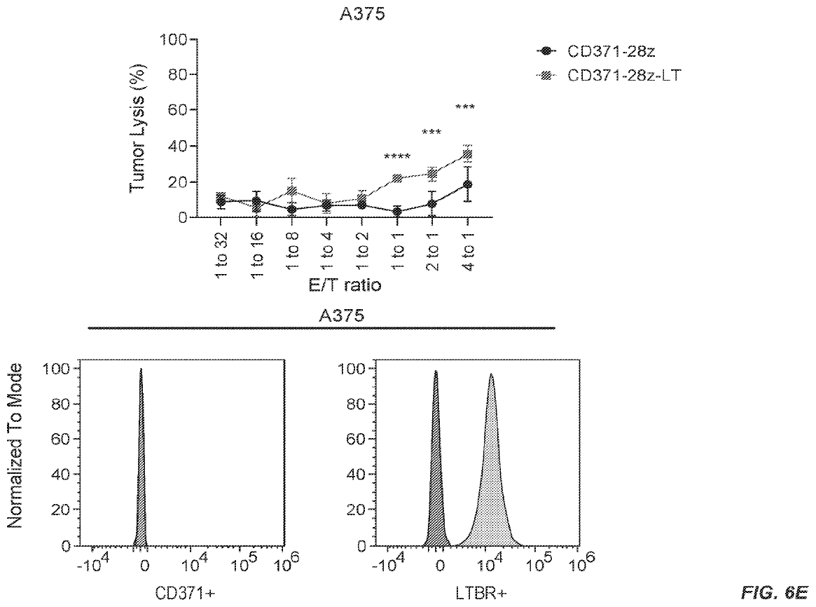

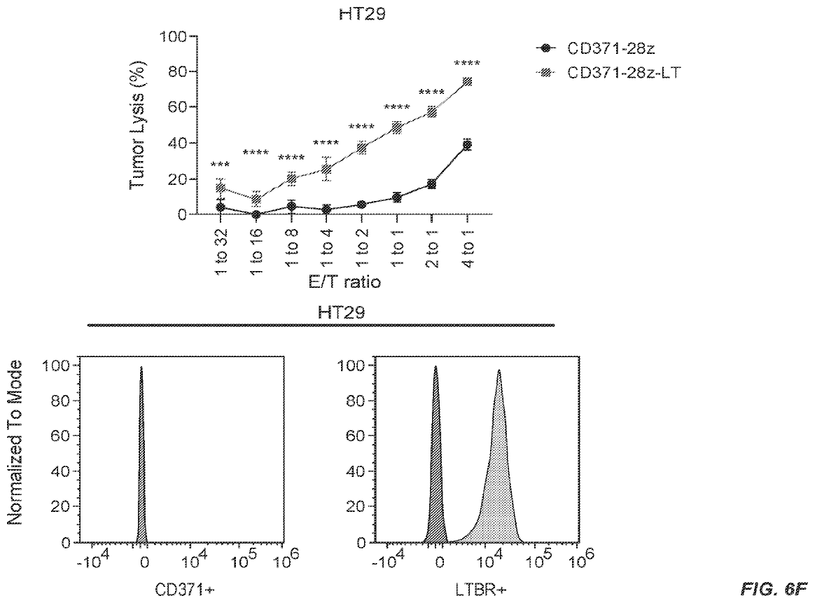

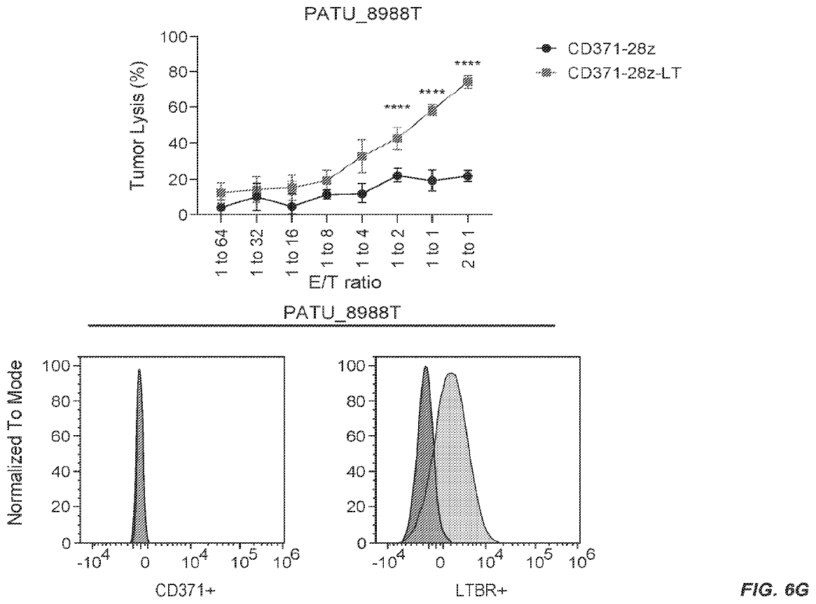

Healthy human donor-derived T cells expressing CD371-target CAR (B10H4L28z or B10H4L28z-LIGHT) were cocultured with tumor cells expressing GFP and firefly luciferase at different effector: tumor ratios. 24 hours later, bioluminescence was measured and plotted as a percentage of the signal detected in a coculture of non-functional CD371-CAR T cells. The results are shown in FIGS. 6A to 6G. As shown in FIG. 6A, no cytolytic differences were observed on U937gL AML cells between B10HL28z (CD371_28z) and B10HL28z-LIGHT (CD371_28z_LIGHT) CAR T cells at a high effector: tumor ratio. As shown in FIGS. 6B to 6D, B10HL28z-LIGHT CAR T cells demonstrated more specific lysis of CD371-low and negative AML cell lines as compared to B10HL28z CAR T cells, which supports the enhanced cytolytic capacity due to the addition of the LIGHT transgene. Conventional CAR T cells without LIGHT and LIGHT-modified CAR T cells exhibited similar cytotoxicity against antigen-positive cell lines in vitro but LIGHT-modified CAR T cells exhibited superior cytotoxicity in an antigen-negative model as compared to the CAR T cells without LIGHT. In addition, B10HL28z-LIGHT prevented outgrowth of antigen-negative solid tumor cell lines, e.g. melanoma cells A375 in FIG. 6E, colorectal adenocarcinoma cells HT29 in FIG. 6F, and pancreatic adenocarcinoma cells 8988t in FIG. 6G, as compared to the efficacy of the CAR T cells without LIGHT, i.e., B10HL28z.

### The Killing Activity of LIGHT Relates to LTβR

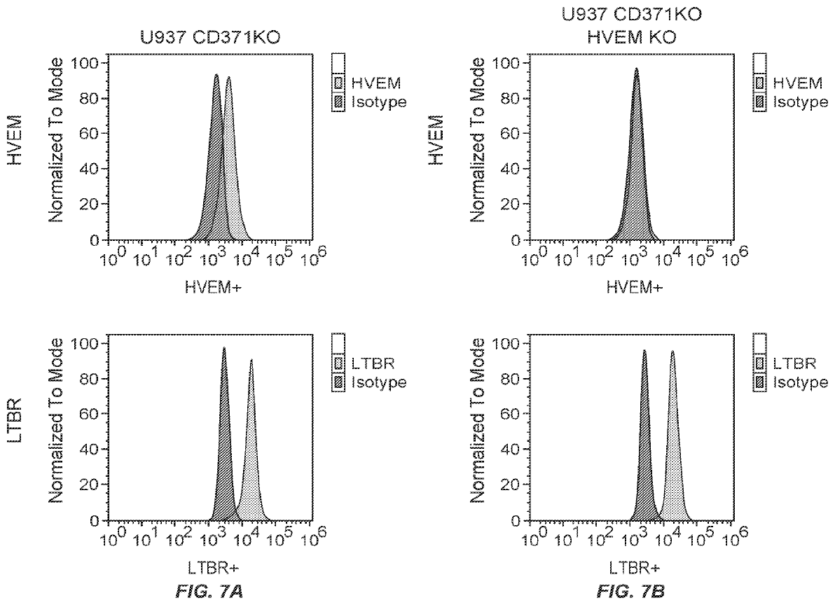

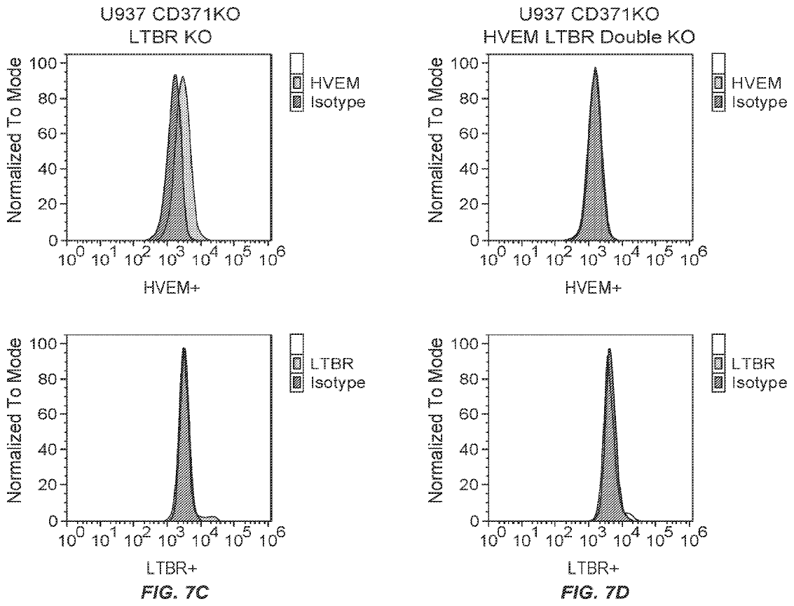

To further validate that the enhanced cytolytic functionality of the CAR T cells was due to LIGHT, one or both receptors for LIGHT: LTβR and HVEM, were knocked out by CRISPR-Cas9 in CD371-knockout U937 AML cell lines (CD371 KO U937 AML as show in FIG. 7A). The knockout cell lines included U937 CD371 KO AML cancer cell lines with LTβR knockout, HVEM knockout, or HVEM+LTβR double knockout. Cells expressing GFP, and firefly luciferase were sorted for no HVEM/LTβR expression and validated by flow cytometry relative to isotype control. Short-term co-culture assays of B10HL28z CAR T cell and the new CRISPR KO cell lines, and B10HL28z-LIGHT CAR T cells and the new CRISPR KO cell lines were performed. Healthy human donor-derived CD371-targeted CAR T cells were cocultured with tumor cells expressing GFP and firefly luciferase at different effector: tumor ratios. 24 hours later, bioluminescence was measured and plotted as a percentage of the signal detected in a coculture of non-functional CD371-CAR T cells. Plots were representative of three independent experiments. Data errors were analyzed with mean±SEM.

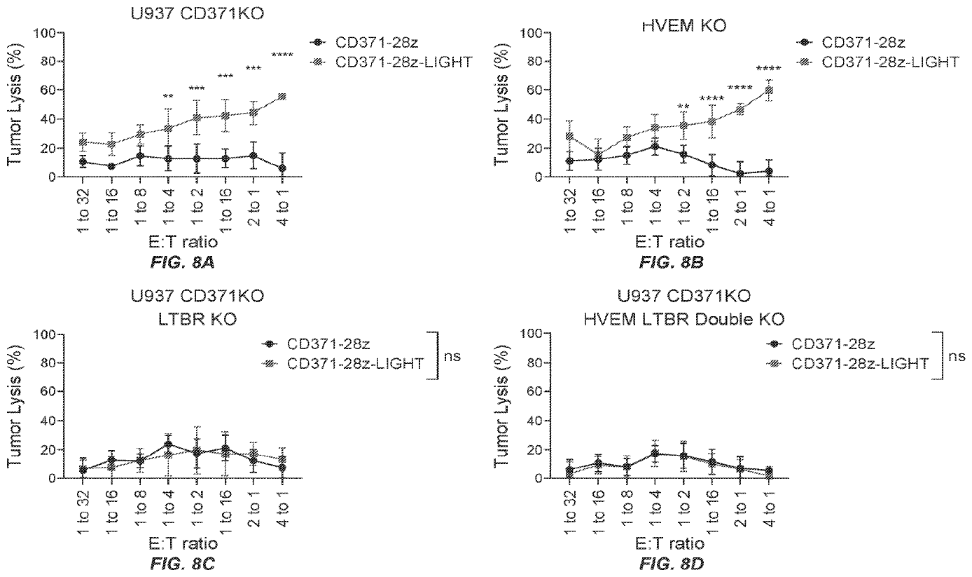

The results in FIGS. 7A to 7D demonstrated that three U937 CD371 KO AML cancer cell lines with LTβR knockout (FIG. 7C), HVEM knockout (FIG. 7B), or HVEM+LTβR double knockout (FIG. 7D) were established. As shown in FIG. 8C, the knockout of LTβR from the tumor cells was observed to eliminate the killing capacity of B10HL28z-LIGHT CAR T cells, while HVEM knockout tumor cells (FIG. 8B) conveyed no resistance to the killing capacity of the CAR T cells. The result suggests that LIGHT from adoptively transferred T cell binds to LTβR receptors on tumor cells and elicits downstream signaling pathways to confer a killing advantage.

### Example 3—In Vivo Anti-Tumor Activities of CD371-Targeted CAR T Cells

To assess in vivo anti-tumor activities of CD371-targeted CAR T cells, a CD371-low AML xenograft model was used.

### Materials and Methods

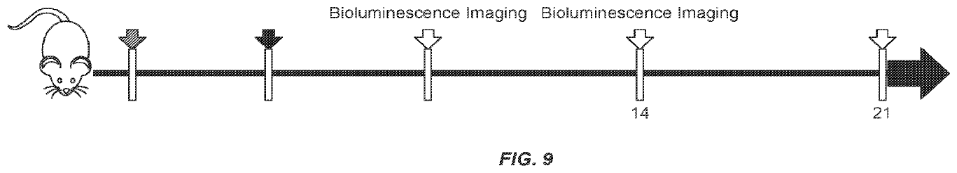

NOD-Prkdc^{em26Cd52}Il2rg^{em26CD22}/NjuCrl, Coisogenic Immunodeficient (NCG) mice were used in xenogeneic studies. All xenograft experiments were performed on 6- to 12-week-old gender-matched mice. Tumors were engrafted with retrovirally transduced Green Fluorescent Protein (GFP)-firefly luciferase transgene and were imaged via bioluminescence to confirm equal tumor load and randomized to different treatment groups one day before CAR T cell treatment. Since the U937 AML cell line highly expressed CD371 and therefore could not be easily discerned functional differences between CD371-targeted CAR and LIGHT-modified CD371-targeted CAR T cells, an acute promyelocytic leukemia cell line NB4 was used for CD371-low AML xenograft studies. 5×10⁵ NB4 AML cells expressing GFP-firefly luciferase were inoculated intravenously 1 day before CAR T cell treatment. Mice were treated with 5×10⁵ CAR T cells intravenously 1 day after tumor inoculation. Mice were euthanized when tumor growth led to a 20% reduction in body weight or when mice suffered from hind limb paralysis and other signs of severe graft versus host disease (GvHD). An experimental layout was illustrated in FIG. 9.

### Results

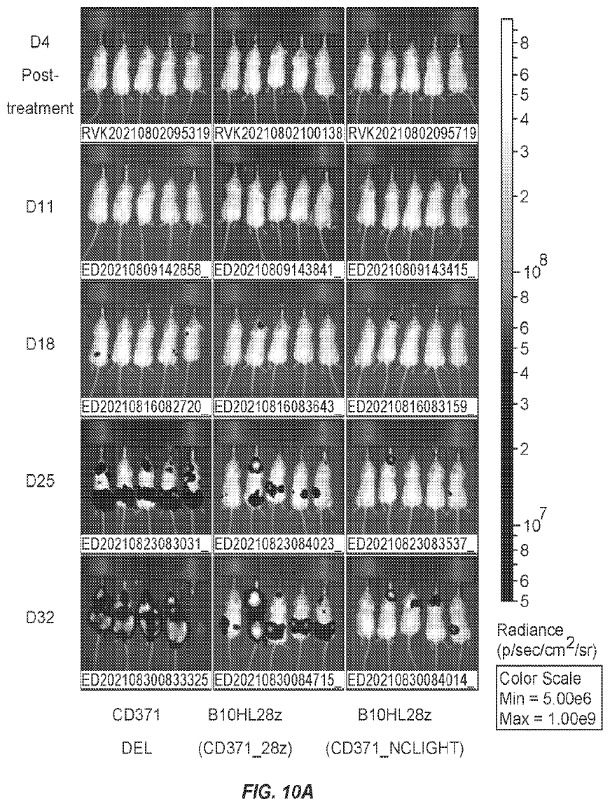

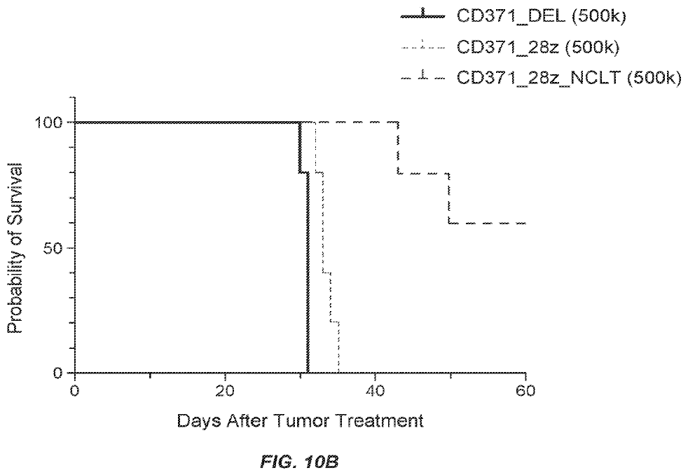

CD371_28z (B10H4L28z) CAR T cells was tested in this xenograft study. CD371_DEL construct served as a negative control without the intracellular signaling domain when the antigen-recognition scFv bound to the target antigen. NCLT represented a non-cleave version of LIGHT. The Tumor burden was tracked with bioluminescence with the addition of D-luciferin substrate and imaged via Spectrum-IVIS machine. Survival was calculated for NCG mice inoculated with 5×10⁵ NB4 cells and treated with 5×10⁵ CAR T cells intravenously the next day (n=5). As shown in FIGS. 10A and 10B, LIGHT-modified CAR T cells controlled tumor outgrowth (FIG. 10A) and improved survival in mice bearing systemic NB4 AML in a xenograft model (FIG. 10B).

### Example 4—In Vitro Cytotoxicity Activities of Mesothelin-Targeted CAT T Cells

To further examine the functions of the LIGHT-modified CAR T cell in solid tumors, human T cells were transfected with the mesothelin-targeted CARs (e.g., “meso_28z”, “meso_28z-LIGHT”, and “meso_28z-NCLIGHT”).

Pancreatic ductal adenocarcinoma (PDAC) cell lines, like AML, express high LTβR. The following PDAC cell lines were used: AsPC1 pancreatic adenocarcinoma cell line, BxPC3 pancreatic adenocarcinoma cell line, CAPAN2 pancreatic adenocarcinoma cell line, MIAPACA2 pancreatic adenocarcinoma cell line, PAN01 pancreatic adenocarcinoma cell line, and PDAC2 Patient-Derived-Xenograft (PDX) cell lines. The following solid tumor cell lines were also tested: JMN2 mesothelioma cell line, MDA-MB-231 triple negative breast cancer cell line, SW620 colorectal cancer cell line, OS69 brain metastasis from osteosarcoma PDX cell line, and TFKIICC cholangiosarcoma PDX cell line. The cells were analyzed for their mesothelin expressions and LTβR expressions before testing the cytotoxic capacity of the mesothelin-targeted CAR T cells overexpressing LIGHT. The number of the surface molecules or antigen density of mesothelin for each cell line was semi-quantitatively determined by the QIFIkit.

Healthy human donor-derived mesothelin-targeted CAR T cells were cocultured with various cells expressing GFP and firefly luciferase at different effector: tumor ratios. DEL construct served as a negative control without the intracellular signaling domain when the antigen-recognition scFv bound to the target antigen. NCLT represented a non-cleave version of LIGHT. 1928z served as a negative control as an irrelevant human CAR T cell construct. PAN01 PDAC cell line that did not express mesothelin and LTβR was used as a negative control. 72 hours later, bioluminescence was measured and plotted as a percentage of the signal detected in a coculture of untransduced T cells. Plots were representative of three independent experiments. Data errors were analyzed with mean±SEM. Associated tumor lysis of tested CAR T cells was calculated. Data was representative of four independent experiments.

### Results

LIGHT-modified CAR T cells prevented outgrowth of antigen-negative tumor cells

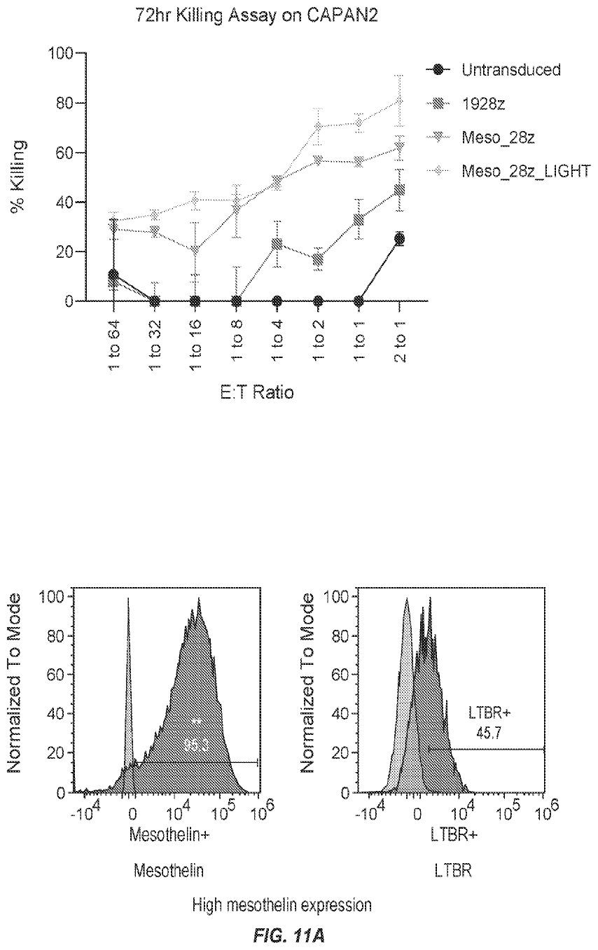

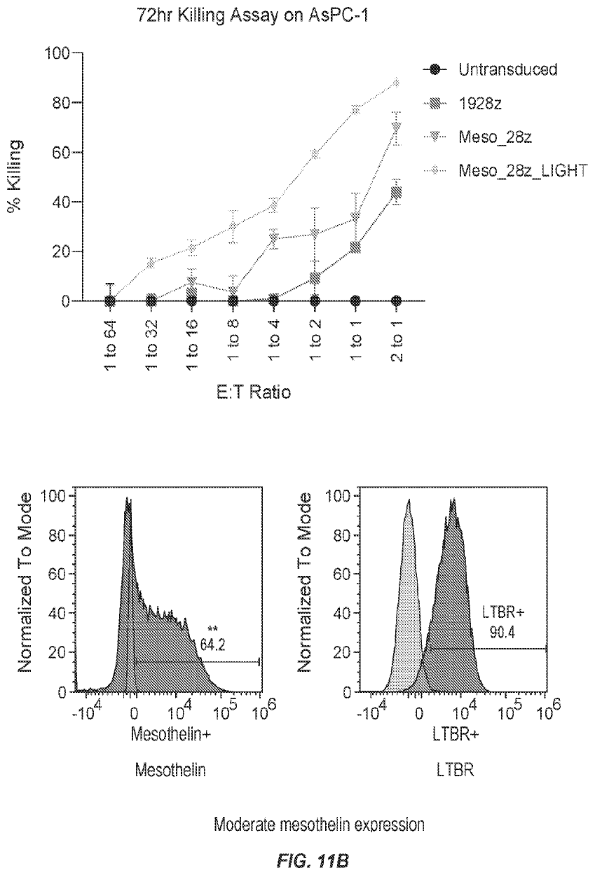

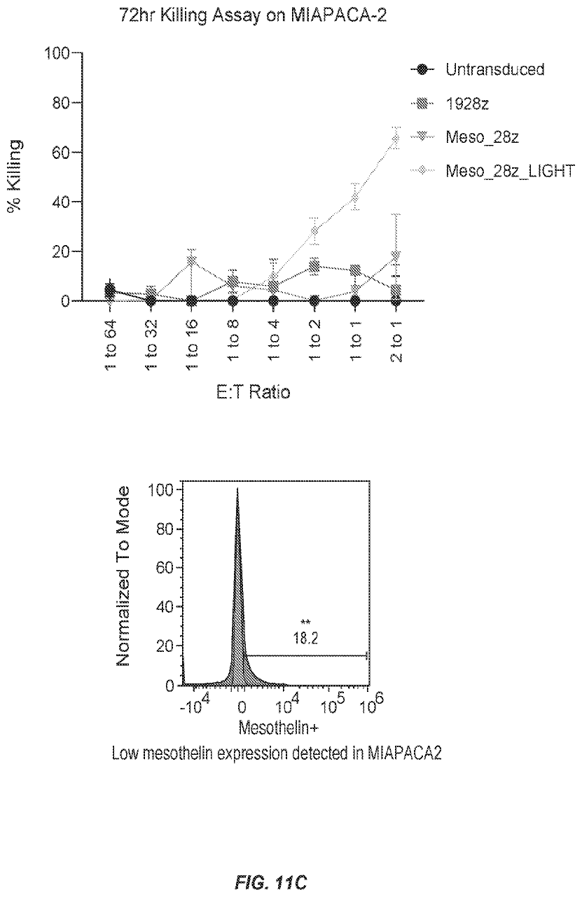

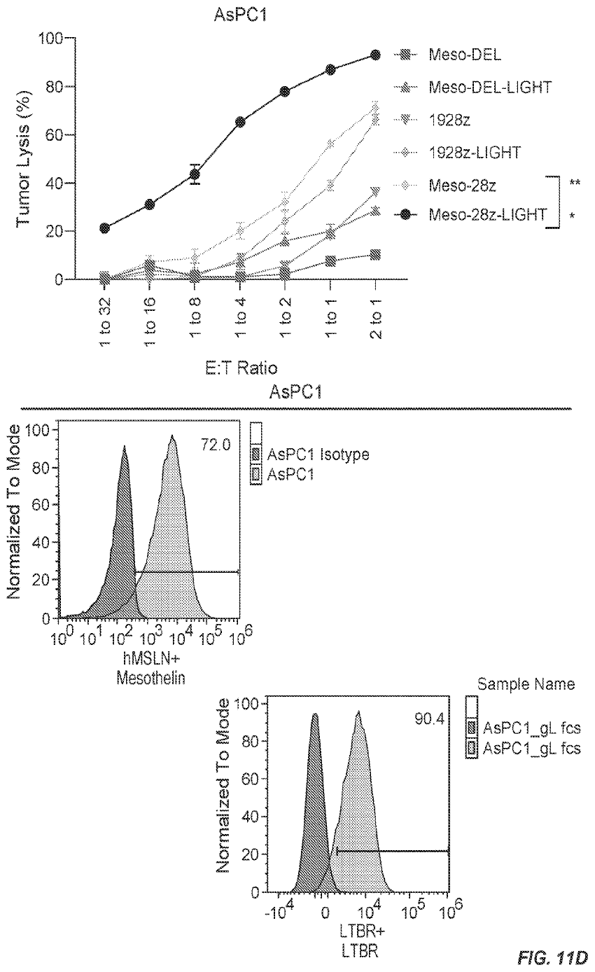

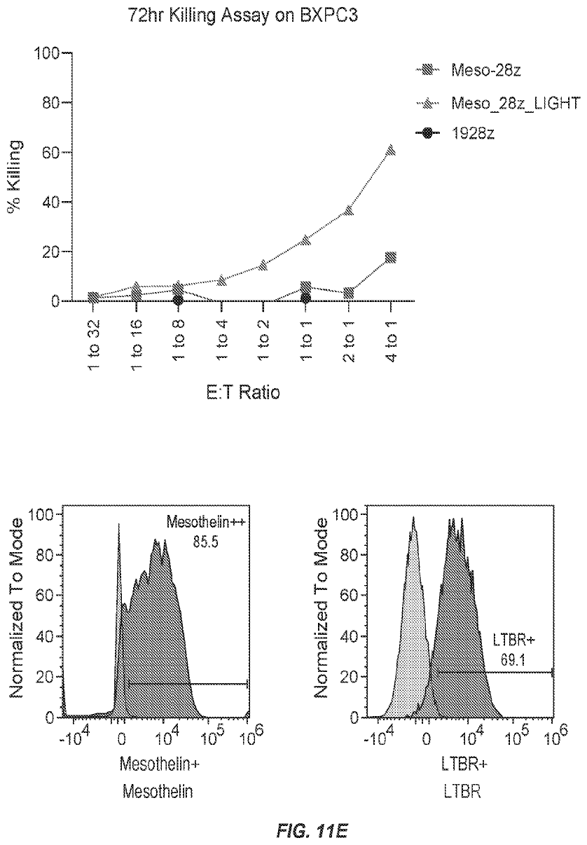

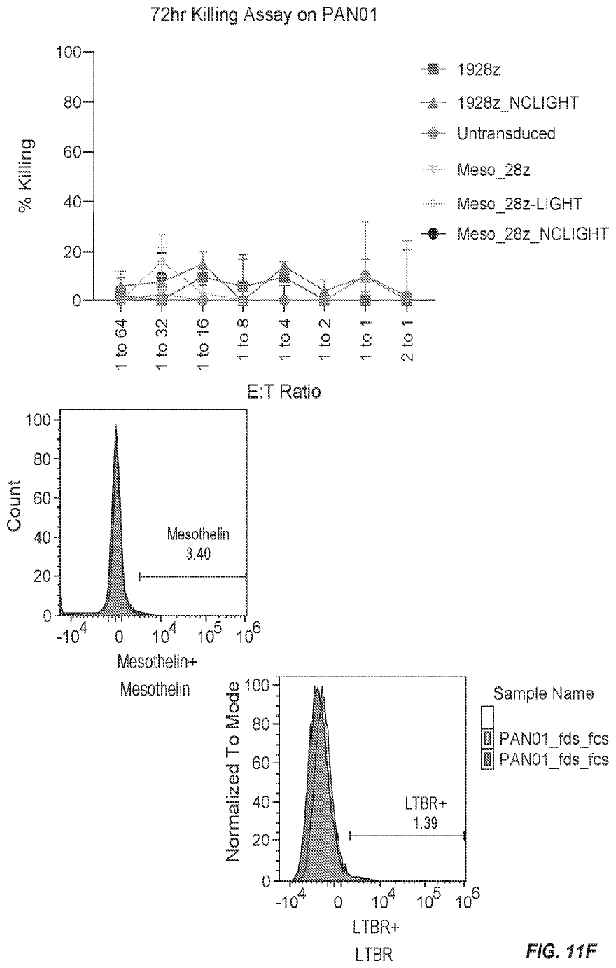

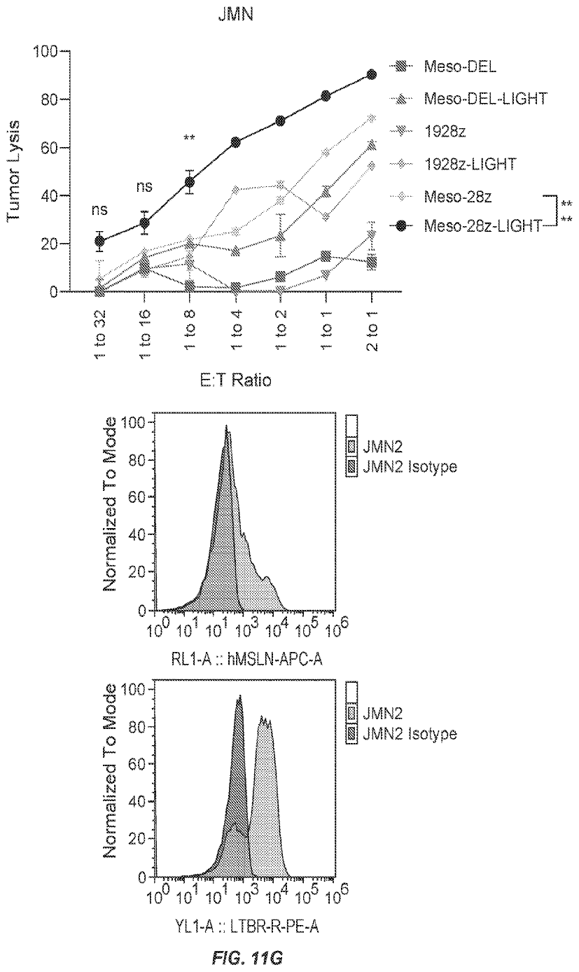

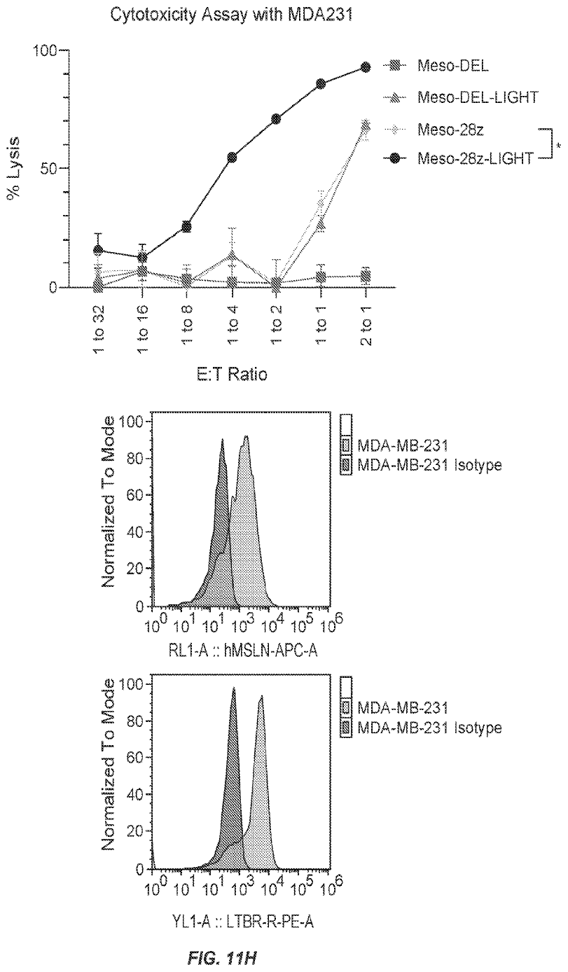

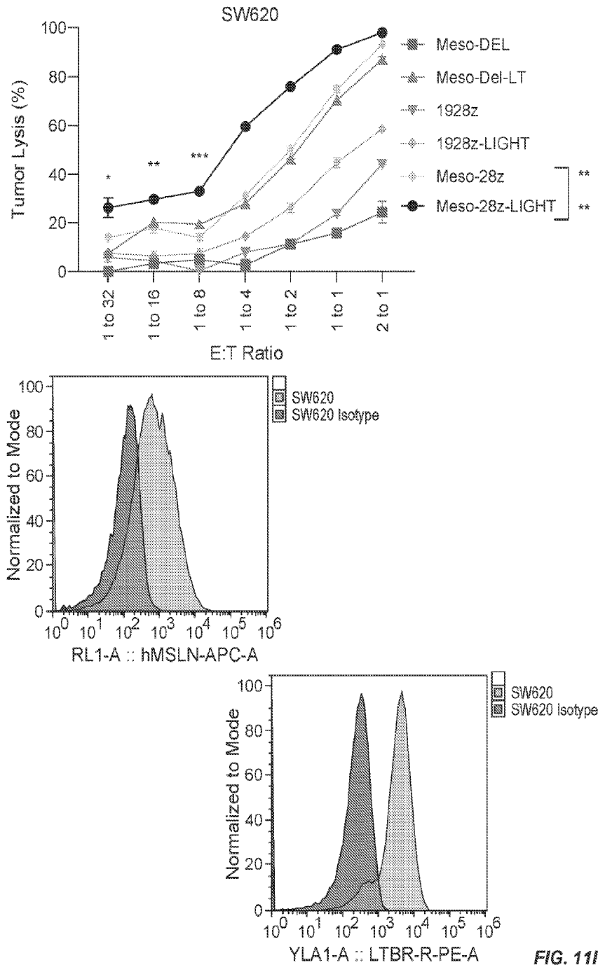

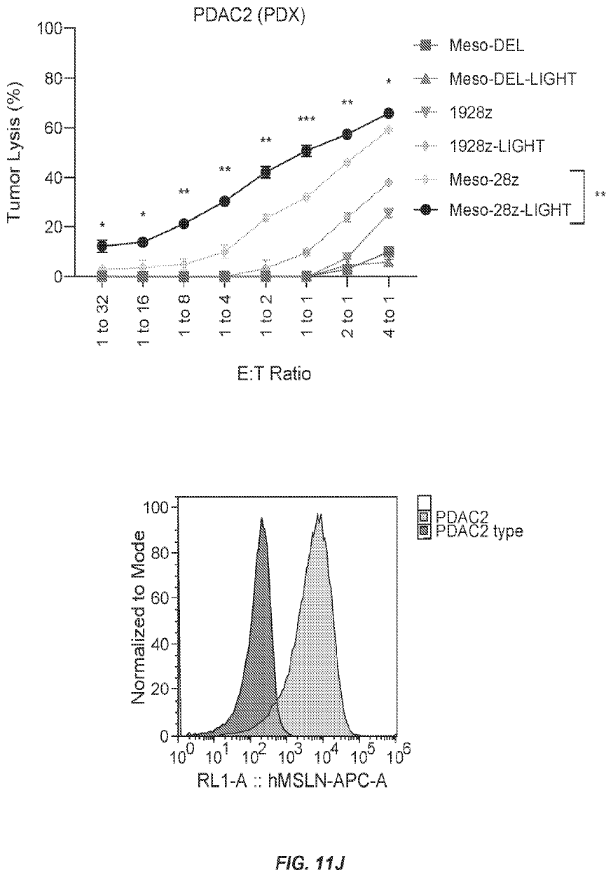

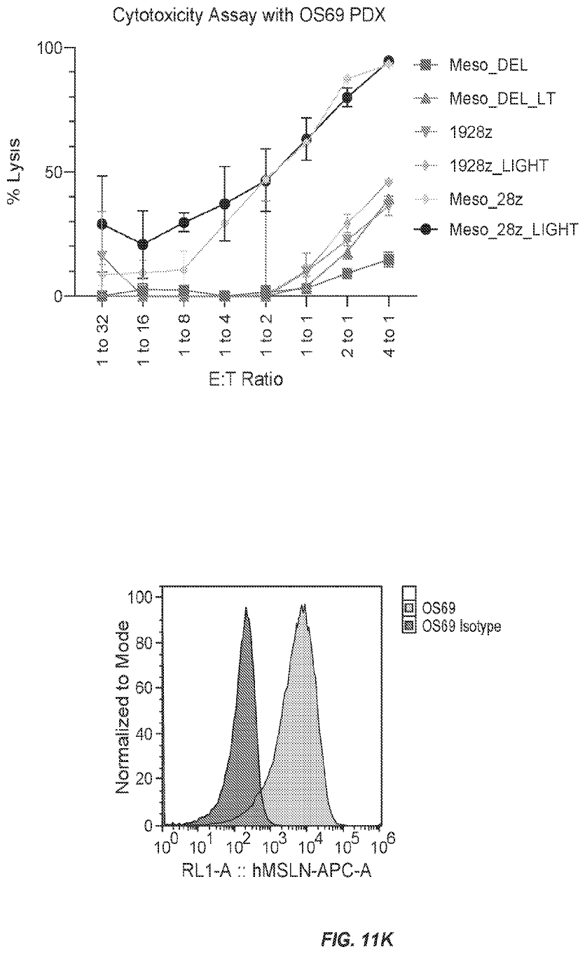

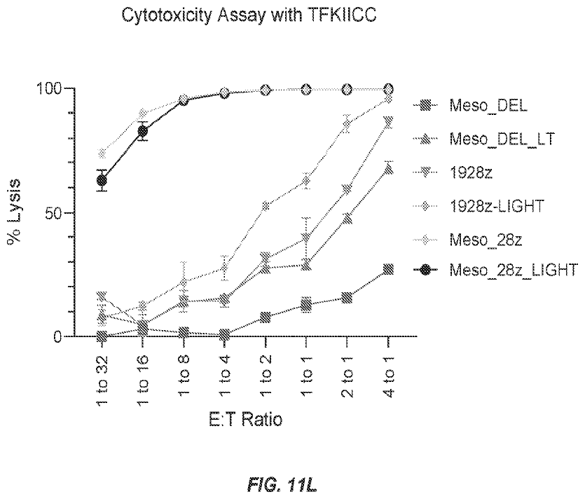

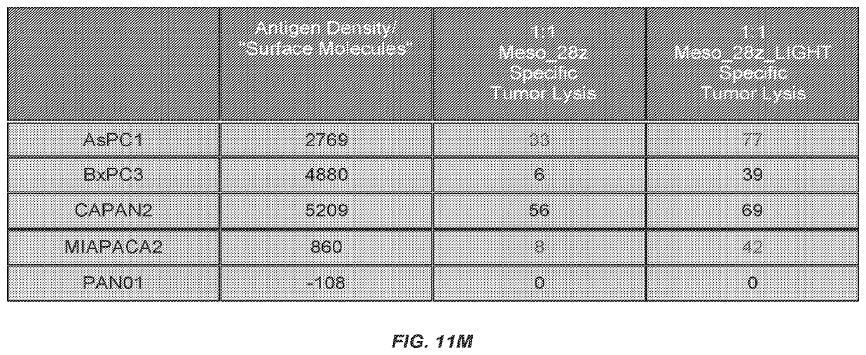

The results in FIGS. 11A to 11M show associated expression of mesothelin and LTβR with the corresponding PDAC cell lines and their respective tumor lysis. As shown in FIG. 11M, high mesothelin expression was detected in CAPAN2 PDAC cell line, AsPC1 was a moderate mesothelin expressing PDAC cell line, MIAPACA2 PDAC cell line exhibited a low mesothelin expression, and PAN01 cell line did not express mesothelin. Furthermore, PAN01 did not express LTβR as shown in FIG. 11F. Consistent with previous findings, in the presence of high antigen density, CAR T cells without LIGHT were able to kill tumor cells effectively. However, when antigen density was low, LIGHT-modified CAR T cells elicited a superior killing advantage compared to conventional CAR T cells without LIGHT, see e.g., FIG. 11B and FIG. 11C.

The results in FIGS. 11G to 11L show associated expression of mesothelin and LTβR with the corresponding solid tumor cell lines and their respective tumor lysis. As shown in FIG. 11G, JMN2 mesothelioma cell line expressed a low level of mesothelin and a moderate level of LTβR. LIGHT-modified CAR T cells exhibited superior killing capacity against mesothelioma in a heterogenous mesothelin expressing model. As shown in FIG. 11H, MDA-MB-231 triple negative breast cancer cell line expressed a moderate level of mesothelin and a moderate level of LTβR. LIGHT-modified CAR T cells exhibited superior killing capacity against breast cancer cell line in a heterogenous mesothelin expressing model. As shown in FIG. 11I, SW620 colorectal cancer cell line expressed a high level of mesothelin and a high level of LTβR. LIGHT-modified CAR T cells exhibit superior killing capacity against colorectal cancer cell line in a heterogenous mesothelin expressing model. As shown in FIG. 11J, PDAC2 patient-derived xenograft (PDX) PDAC cell line expressed a high level of mesothelin. LIGHT-modified CAR T cells exhibited superior killing capacity against PDX PDAC cells. As shown in FIG. 11K, OS69 brain metastasis from osteosarcoma PDX cell line expressed a high level of mesothelin. LIGHT-modified CAR T cells exhibited superior killing capacity against PDX osteosarcoma cells. Lastly, as shown in FIG. 11L, LIGHT-modified CAR T cells exhibited superior killing capacity against TFKIICC cholangiosarcoma PDX cells.

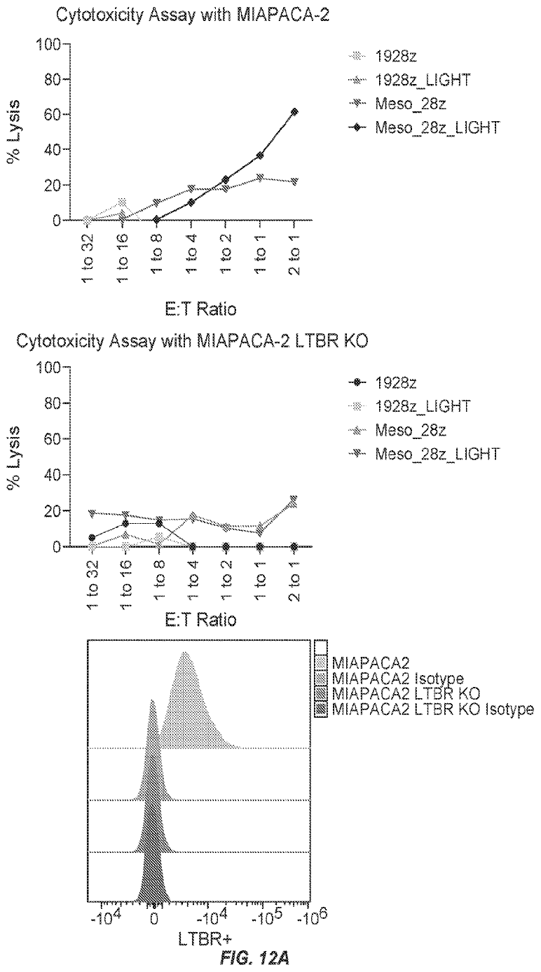

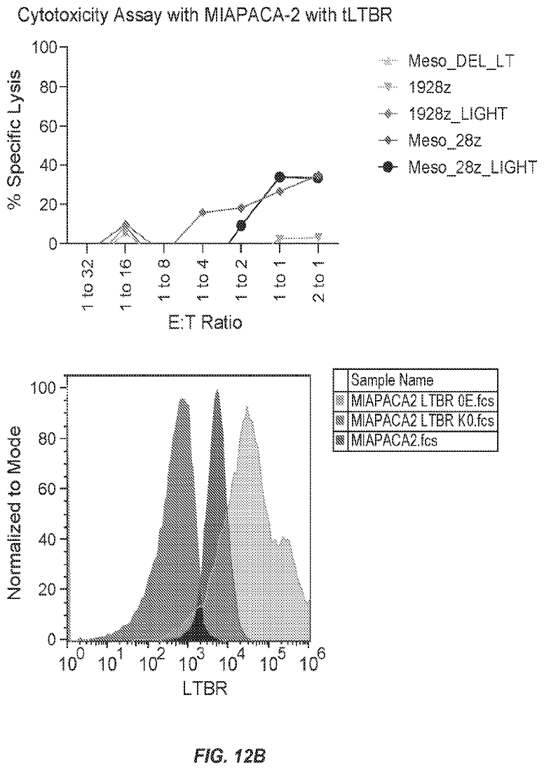

Furthermore, it was observed that the killing capacity of meso_28z LIGHT CAR T cells relate to LTβR, as no killing capacity of meso_28z LIGHT CAR T cells was observed for tumor cells with LTβR-knockout (“LTβR KO”) or truncated LTβR without intracellular signaling domain on cancer cells (“tLTβR”). The truncated LTβR comprises amino acids 31 to 248 of SEQ ID NO: 54 (does not comprise amino acids 249 to 435 of SEQ ID NO: 54). See FIG. 12A and FIG. 12B. As shown in FIG. 12B, truncated LTβR was overexpressed to LTβR KO PDAC cell Lines. The results in FIG. 12A and FIG. 12B demonstrate that truncated LTβR or LTβR-knockout abolished killing advantage of LIGHT-modified CAR T cells. The results support that LIGHT from adoptively transferred T cell binds to LTβR on tumor cells and elicits downstream signaling pathways to confer a killing advantage.

### LIGHT-Modified Mesothelin-Targeted CAR T Cell Exhibited Similar Proliferation

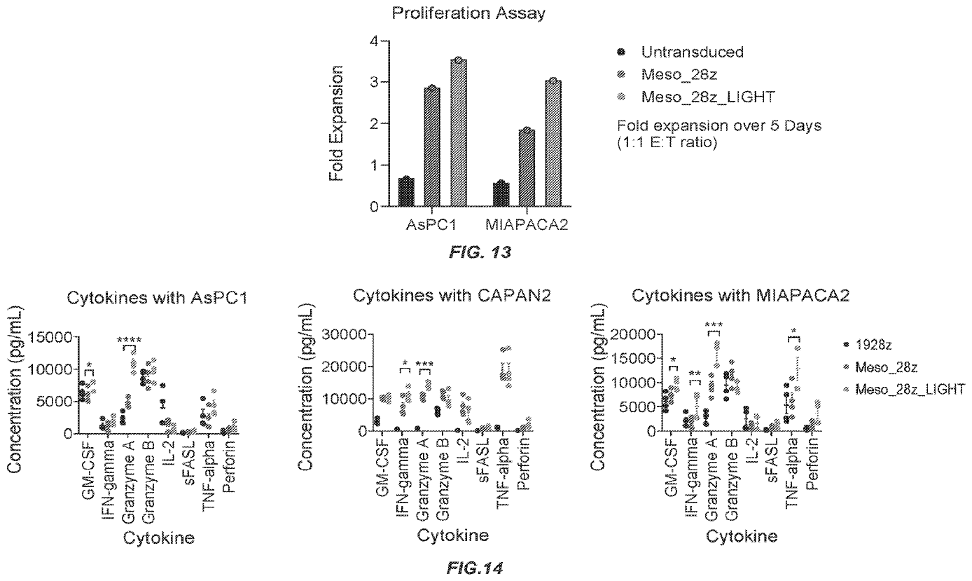

The T cell proliferation was assessed in the PDAC model. CAR T cells were cocultured with tumor cells at a 1:1 effector: target ratio for 5 days. The fold expansion was relative to the initial amount of CAR T cells added after 5 days of co-culture. As shown in FIG. 13, LIGHT-modified mesothelin-targeted CAR T cell exhibited similar proliferation in a coculture assay with mesothelin-positive PDAC cell lines.

### LIGHT-Modified CAR T Cells Demonstrated Superior Proinflammatory Cytokines Secretion

The cytokine secretion profile of LIGHT-modified CAR T cells was characterized and the relation of cytokine levels with the superior cytolytic capacity of the CAR T cells was examined. For in vitro cytokine production, 5×10⁴ CAR T cells and 5×10⁴ tumor cells were co-cultured in a 96-well round-bottom plate in 200 μL of T cell media. After 24 hours, the supernatant was collected and analyzed using the FLEXMAP 3D system (Luminex). Data representative of four separate donors and data errors were analyzed with mean±SEM.

As shown in FIG. 14, meso_28z_LIGHT CAR T cells produced more proinflammatory cytokines such as GMCSF, IFN-γ, and TNF-α. Surprisingly, vastly higher levels of granzymes A were secreted from these CAR T cells, which was a phenotype that had not been previously reported or appreciated to have any cytotoxic effect by CAR T cells. Granzyme B and perforin were appreciated to be the dominant factors for caspase-dependent apoptosis of tumor cells, while granzyme A was reported to play a role in caspase-independent death.

### Example 5—In Vivo Anti-Tumor Activities of Mesothelin-Targeted CAR T Cells

To assess in vivo anti-tumor efficacy of mesothelin-targeted CAR T cells with LIGHT, a xenograft flank model with the AsPC1 PDAC cell line and an antigen-low xenograft flank model with the MIAPACA2 PDAC cell line were used.

### Materials and Methods

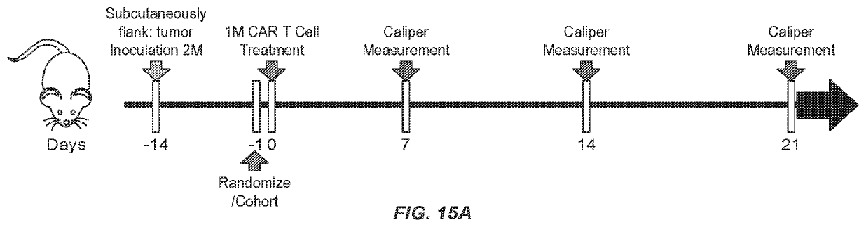

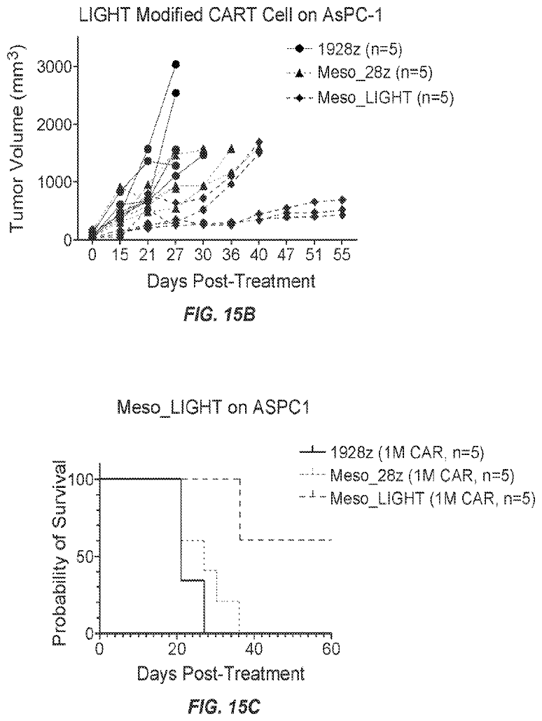

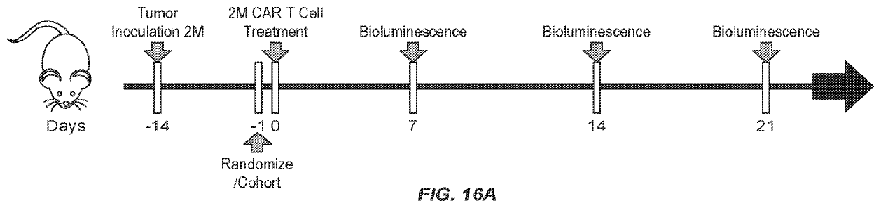

NOD-Prkdc^{em26Cd52}Il2rg^{em26CD22}/NjuCrl, Coisogenic Immunodeficient (NCG) mice were used in xenogeneic studies. All xenograft experiments were performed on 6- to 12-week-old gender-matched mice. Tumors were engrafted with retrovirally transduced Green Fluorescent Protein (GFP)-firefly luciferase transgene and were imaged via bioluminescence to confirm equal tumor load and randomized to different treatment groups one day before CAR T cell treatment. AsPC1 cell line was chosen due to good in vitro killing and expansion of the LIGHT-modified CAR T cells. NCG mice were subcutaneously injected with 2×10⁶ AsPC1 cells. Fourteen (14) days later, mice were randomly assigned to four groups and were infused intravenously with 1×10⁶ CAR T cells. Tumor burden was measured by caliper at indicated days since CAR T cell infusion. Tumor burden (mm³) was quantified by (L×W×W)/2 in mice treated with various CAR T cell constructs at indicated days since CAR T cell infusion. An experimental layout of AsPC1 xenograft flank model was illustrated in FIG. 15A. Mouse survival days post CAR T cell treatment were shown by Kaplan-Meyer plot.

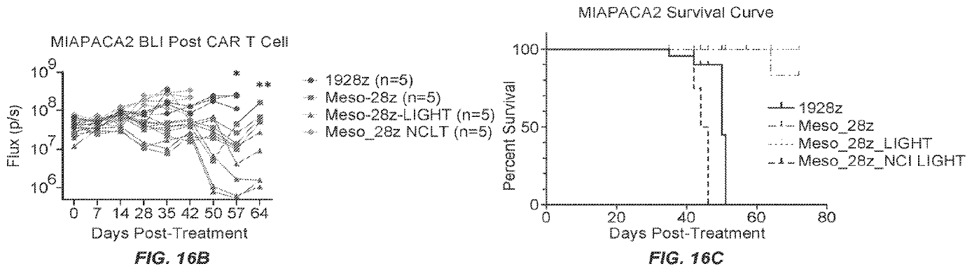

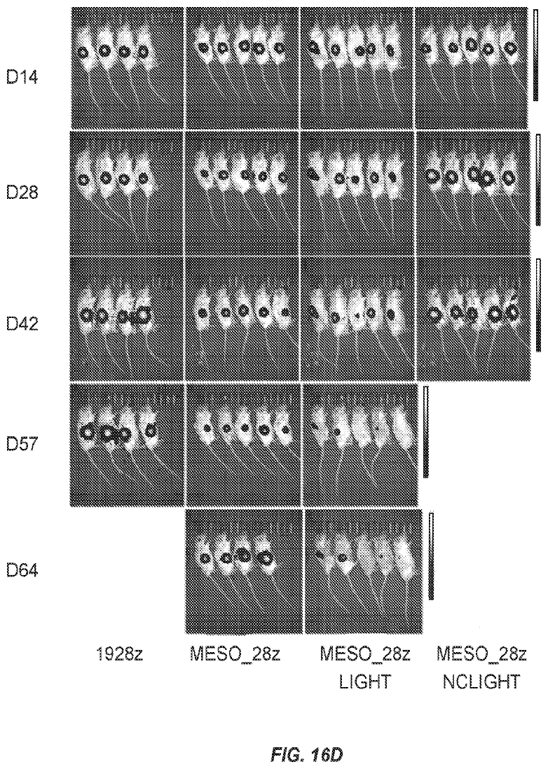

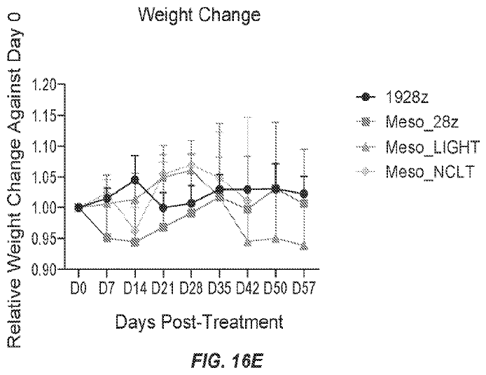

In addition, MIAPACA2 representing a low mesothelin expressing cell line was tested. NCG mice were subcutaneously injected with 2×10⁶ MIAPACA2 cells. Fourteen (14) days later, mice were randomly assigned to four (4) groups and were infused intravenously with 2×10⁶ CAR T cells. Tumor burden was measured by bioluminescence at indicated days since CAR T cell infusion. Tumor burden (total flux) was quantified by photons/s in mice treated with various CAR T cell constructs at indicated days since CAR T cell infusion. An experimental layout of MIAPACA2 xenograft flank model was illustrated in FIG. 16A. Mouse survival days post CAR T cell treatment were shown by Kaplan-Meyer plot. Bioluminescence images of MIAPACA2 tumor cells in vivo after CAR T cell treatment were collected. Toxicity was tested for the CAR T cell treatment. The relative body weight change compared to the Day 0 treatment was plotted.

### Results

Meso_28z CAR T cells, meso_28z-LIGHT, and meso_28z_NCLIGHT were tested in the xenograft studies. CAR T cells 1928z were used as an irrelevant CAR T cell control. In the xenograft flank model with the AsPC-1 PDAC cell line, as shown in FIG. 15B, tumor volume tracked by caliper measurement showed that meso_28z-LIGHT CAR T cells controlled tumor outgrowth. The results in FIG. 15C demonstrated that meso_28z-LIGHT CAR T cells significantly improved overall survival as compared to the conventional meso_28z CAR T cells without LIGHT. In the xenograft flank model with the MIAPACA2 PDAC cell line, MIAPACA2 was used because MIAPACA2 cell expressed a low level of mesothelin but exhibited susceptibility to LIGHT-modified CAR T cells in vitro characterization. Consistent with the in vitro data, MIAPACA2 flank xenograft models demonstrated superior tumor burden control exhibited by meso_28z-LIGHT CAR T cells, as seen by the bioluminescence of the tumor (see FIG. 16B). The meso_28z-LIGHT CAR T cells also exhibited a survival benefit as compared to CAR T cells without LIGHT (see FIG. 16C). Representative bioluminescence imaging of tumor burden tracked weekly showed a reduction in tumor burden with meso_28z-LIGHT CAR T cells (see FIG. 16D). Since previous studies showed that constitutively expressing LIGHT had a hyper-activated T cell population putting them at risk for autoimmunity (Brunetti et al., Front Immunol, 2018, 9:2459), the mice were examined for observable toxicity via weight change throughout the in vivo experiments. As shown in FIG. 16E, no noticeable weight change associated with LIGHT-modified CAR T cells was observed in the xenograft models.

### Example 6

This Example is an updated report of Examples 1 to 5.

### Methods and Materials

### Animal Models

For xenogeneic studies, NOD-Prkdc^{em26Cd52}Il2rg^{em26CD22}/NjuCrl, Coisogenic

Immunodeficient (NCG) mice were purchased from Charles River Laboratories and subsequently bred and housed under specific-pathogen-free (SPF) conditions. For all xenograft experiments, 6- to 12-week-old gender-matched mice were used. Tumors were engrafted with retrovirally transduced green fluorescent protein (GFP)-firefly luciferase transgene, and mice were imaged via bioluminescence to confirm equal tumor load and randomized to different treatment groups 1 day before CAR T cell treatment. For the xenograft model of PDAC, 2×10⁶ AsPC1 or MIAPACA2 PDAC cells expressing GFP-firefly luciferase were inoculated subcutaneously 14 days prior to CAR T cell treatment. Mice were treated with 1×10⁶ CAR T cells for AsPC1 and 2×10⁶ CAR T cells for MIAPACA2 intravenously 14 days after tumor inoculation. Mice were euthanized when tumor volume exceeded 1,500 mm³ by caliper measurements, when tumor growth led to a 20% reduction in body weight, or when mice suffered from hind limb paralysis and other signs of (GvHD). The investigator was blinded when assessing the outcome. For PDX models of PDAC, NCG mice were inoculated with 2×10⁵ cells orthotopically (intrapancreatic) for 7 days prior to treatment of 4×10⁵ CAR T cells. For syngeneic mouse models, C57BL/6J (RRID:IMSR_JAX:000664) were purchased from Jackson Laboratory. 6 to 12 weeks old gender-matched mice were used to assess the toxicity profile of LIGHT-CAR T cells in an immunocompetent model.

### Cell Lines

293-Glv9-packaging cells were maintained in DMEM (Dulbecco's Modified Eagle Medium) with high-glucose supplemented with 10% heat-inactivated fetal bovine serum (FBS), nonessential amino acids (Atlanta Biological Flowery Branch), 2 mM L-glutamine (Invitrogen), and 1% penicillin/streptomycin (Invitrogen). The U937 human acute leukemia line, the AsPC1 pancreatic adenocarcinoma cell line, the CAPAN2 pancreatic adenocarcinoma cell line, the MIAPACA2 pancreatic adenocarcinoma cell line, PATU8988t and Panc1 pancreatic adenocarcinoma cell line, JMN, SW620, and MDA-MB-231 were modified to express GFP-firefly luciferase to detect cancer cells in vitro and in vivo by bioluminescence. All cancer cell lines were maintained in RPMI-1640 medium or DMEM supplemented with 10% heat-inactivated FBS nonessential amino acids (Atlanta Biological Flowery Branch), 10 mM HEPES (hydroxyethyl piperazineethanesulfonic acid, Invitrogen), 2 mM L-glutamine (Invitrogen), 1% penicillin/streptomycin (Invitrogen), and 11 mM glucose (Invitrogen). Cancer lines were sorted by fluorescence-activated cell sorting (FACS) based on the high expression of GFP. Cell lines were authenticated with short tandem repeat (STR) profiling and routinely tested for potential mycoplasma contamination (Lonza Mycoalert Mycoplasma Detection Kit).

### Generation of Retroviral Constructs

Plasmids encoding the CAR constructs in the SFG gamma-retroviral vector were transfected into gpg29 fibroblasts (H29) with human signaling domains. The calcium phosphate (CaPO₄) ProFection Mammalian Transfection System (Promega) was used according to the manufacturer's suggested protocol to generate vesicular stomatitis virus G-glycoprotein-pseudo typed (VSV-G) retroviral supernatants. The VSV-G retroviral supernatants were then used to transduce stable retroviral producer line 293-Glv9. The SFG gamma-retroviral vector was cloned by Gibson Assembly (New England Biolabs) using a designed gBlocks gene fragment (Integrated DNA Technologies) that includes anti-human CD371 and anti-human mesothelin ScFv, Myc-tag sequence (EQKLISEEDL), human CD28 transmembrane and intracellular domain, human CD3ζ intracellular domain without the stop codon, P2A-self cleaving peptide, and the human LIGHT protein. In addition, VSV-G retroviral supernatants were used to construct stable Moloney murine leukemia virus-pseudotyped retroviral particle-producing Phoenix-ECO cell lines.

### Isolation and Culture of Primary Human T Cells

Regular buffy coats containing peripheral blood from de-identified healthy donors were collected. Peripheral blood mononuclear cells (PBMC) were isolated from buffy coats using Lymphoprep (Stemcell) gradient centrifugation. Purified T cells were isolated by magnetic negative selection CD3 using the EasySep Human T Cell Isolation Kit (Stemcell). Immediately after isolation, T cells were resuspended in T cell medium (RPMI-1640+10% heat-inactivated FBS+2 mM L-Glutamine+1% Penicillin/streptomycin) in addition to IL-2 (100 IU/mL) and CTS CD3/CD28 Dynabeads at a bead to cell ratio of 1:2 (Thermo Fisher Scientific). Forty-eight hours after the initial expansion, T cells were spinoculated with retroviral supernatant collected from 293Glv9 retroviral packaging cells on RetroNectin-coated plates (Takara Bio) for 2 consecutive days. Transduction efficiency was determined by flow cytometric analysis. All experiments were normalized for CAR+viable cells.

### Mouse T Cell Isolation and Retroviral Transduction

Mice were euthanized and their spleens were harvested. Following tissue dissociation and red blood cell lysis. CD3⁺ T cells were enriched via negative selection using the EasySep Mouse T Cell Isolation Kit (StemCell). Cells were then expanded in vitro by culturing in RPMI-1640 supplemented with 10% heat-inactivated FBS, nonessential amino acids, 1 mM sodium pyruvate, 10 mM HEPES, 2 mM L-glutamine, 1% penicillin/streptomycin, 11 mM glucose, 2 μM 2-mercaptoethanol, 100 IU of recombinant human IL-2 (Prometheus Therapeutics & Diagnostics), and anti-CD3/28 Dynabeads (Life Technologies) at a bead: cell ratio of 1:2. 24 h and 48 h after initial expansion, T cells were spinoculated with viral supernatant collected from Phoenix-ECO cells. After the second spinoculation, cells were rested for one day and then used in adoptively transfer studies.

### Short-Term Quantitative Cytotoxicity Assay

The short-term cytotoxicity of CAR T cells was assessed by a luciferase-based coculture assay with target cells expressing firefly-luciferase. Target tumor cells (5×10⁴) and effector CAR T cells were incubated with varying (E: T) ratios in triplicates in white/black-walled 96-well plates (Corning) in a total volume of 200 μL of T cell media. Target tumor cells were plated alone at the same cell density to determine the maximal luciferase signal without CAR T cells. After 24 hours or 72 hours differing by cell type, 75 ng of D-luciferin (Gold Biotechnology) was dissolved in 50 μL of PBS and added to each well using multichannel pipettes. The bioluminescence reading of each well was detected using a Spark plate reader (Tecan) and quantified using the SparkControl software (Tecan). Percent cell lysis was determined by the proportion of the sample signal divided by the max signal (target tumor cells alone).

### Repetitive Antigen Stress-Test Assay (Proliferation Assay)

CAR T cells were co-cultured with target tumor cells at E:T ratio of 4:1 in 12-well triplicates. After 5 days, the cells were collected, and a portion of the population was stained for CAR expression and analyzed via flow cytometry. CAR T cells were counted via myc tag, while tumor cells were evaluated as GFP-positive populations. CAR T cells were replated back to the original E: T ratio to new tumor target cells for subsequent rounds of coculture. Total fold expansion was calculated by multiplying the fold expansion in each round of stimulation.

### Adoptive Transfer of CAR T Cells

For tracking tumor studies, immunocompromised mice were inoculated intravenously (IV) with firefly luciferase-expressing tumor cells on day 0. Bioluminescence imaging used the IVIS Spectrum in vivo imaging system with Living Image software, RRID:SCR_014247 (PerkinElmer) for the acquisition of imaging datasets. Mice with equal tumor burden of mice were randomized to different cohorts at the time of CAR T cell treatment. CAR T cells (1-2×10⁶) IV were used in subcutaneous flank models with 14 days of PDAC tumor engraftment. 4×10⁵ CAR T cells i.v were used for orthotopic intrapancreatic PDX models 7 days after tumor engraftment.

### In Vitro T Cell Stimulation Assay

To assess the costimulation potential of LIGHT, ULTRA-LEAF anti-CD3 antibody (OKT3, BioLegend) resuspended in 50 μL of sterile PBS was plated on a non-tissue coated 96-well plate overnight at 4° C. Plates were washed 2× with sterile PBS before the addition of healthy donor T cells isolated from PBMC (EasySep Human T Cell Isolation Kit, Stem Cell). Media containing recombinant human IL-2 (100 IU/mL) and varying concentrations of soluble recombinant LIGHT (RNDsystems) and recombinant hLIGHT-FC (Sinobiological) was added to assess for an increase in cell number and expression of IL2RA/CD69 via flow cytometry.

### Cytokine Secretion Profiling (Luminex)

After co-incubation of cancer cells with CAR T cells for 24 hours at E:T ratio (25k/25k) in 96-well round bottom plate in 200 μL of T cell media, culture supernatants were collected and analyzed. Cytokine detection was done using the MILLIPLEX MAP Human Cytokine/Chemokine, Premixed 9 Plex Kit (Millipore) and the FLEXMAP 3D system (Luminex).

### CRISPR-Cas9-Mediate Knockout of Tumor Cells

U937, AsPC1, and MIAPACA2 cells were transfected by electrotransfer of modified Cas9 mRNA (tri-link) and gRNA (Synthego) using an AgilePulse MAX system (Harvard Apparatus). Cells (2×10⁵) were mixed with 5 μgs of Cas9 mRNA and 5 μgs of gRNA into a 2 mm cuvette. Following electroporation, cells with electroporation buffer were transferred into media and incubated at 37° C., 5% CO2 overnight. Cells were spun down and replaced with fresh culture media. After 72 hours, the knockout efficiency was assessed by surface expression of the target molecule via flow cytometry. The bottom 10% of the knockout populations were sorted out and expanded to generate a bulk cell line encompassing no expression of the target antigen.

LTβR gRNA comprises the RNA sequence set forth in SEQ ID NO: 55, which was provided below:

| [SEQ ID NO: 55] | |
|---|---|
| | UGGUUCUCCGACGCAUAUGG |

HVEM gRNA comprises the RNA sequence set forth in SEQ ID NO: 56, which was provided below:

| [SEQ ID NO: 56] | |
|---|---|
| | AAGGAGGACGAGUACCCAGU |

### Flow Cytometry and FACS Sorting

Flow cytometric analyses were performed using a Beckman Coulter Gallios or a Thermo Fisher Attune NxT flow cytometer. DAPI (0.5 mg/mL, Sigma-Aldrich) or a LIVE/DEAD Fixable Violet Dead Cell Stain Kit (Thermo Fisher) were used to exclude dead cells in all experiments. Human TruStain FcX Fc Receptor Blocking Solution (BioLegend) was used to block the non-specific binding of antibodies via FC receptors. The following anti-human antibodies were used for flow cytometry: anti-CD3e (OKT3/UCHT1), anti-CD4 (SK3), anti-CD8 (SK1), anti-CD11b (M1/70), anti-CD19 (1D3), anti-CD25 (BC96, M-A251), anti-CD45 (HI30/2D1), anti-CD69 (L78/FN50), anti-CD80 (2D10), anti-CD86 (IT2.2), anti-CD371 (50C1), anti-HLA DR (L243), anti-myc (9B11), anti-TIM3 (F38-2E2), anti-LAG-3 (3DS223H), anti-PD1 (J105), anti-LIGHT (T5-39), anti-HVEM (122), anti-LTβR (31G4D8), and CellTrace Far Red dye (C34564).

### Evaluation of Mesothelin Surface Densities

The surface densities of mesothelin molecules on the cancer cells were examined by standardized flow cytometry using a commercial quantitative analysis kit, QIFIKIT® (Agilent) following the user manual provided by the manufacturer of the kit. Briefly, the following parameters were set up and optimized: the voltages for forward-scattering (FSC), side-scattering (SSC), and BLI fluorescence channel (for fluorescein isothiocyanate, FITC) in Attune NxT Flow Cytometer (Invitrogen) using the set-up beads (QIFIKIT®). Next, following standard staining and washing procedures, the calibration beads (QIFIKIT®) were stained. The calibration beads were a combination of 5 populations of beads bearing different known numbers of mouse antibodies on their surfaces, with FITC-conjugated goat anti-mouse immunoglobulin F(ab′)2 fragment. Then, using flow cytometry, the mean fluorescence intensities (MFIs) of the calibration beads of known surface densities, represented by antigen binding capacity (ABC) values, were recorded, from which a standard curve between the MFI and ABC values was constructed.

### ELISA

Supernatants from CAR T cells (3×10⁶/6 well) were collected after 48 hours. ELISA was performed by LIGHT/TNFSF14 Human ELISA Kit (Invitrogen) following the user manual provided by the manufacturer of the kit.

### Sample Preparation for CITEseq

CAR T cells were sorted for myc expression to generate a pure CAR T cell population for convenience of downstream sequencing analysis. After 48-hours co-culture with cancer cells, the CAR T cell population was sorted out and cells were individually resuspended in 100 μL of cell staining buffer (BioLegend) with 5 μL of Human TruStain FcX Fc blocking reagent (BioLegend). Cell suspensions were then incubated at 4° C. for 15 minutes, during which the antibody pool was prepared using 1 μg of each TotalSeq-C Human Universal Cocktail V1.0 antibodies (BioLegend). In addition, Human Totalseq Hashtag 1-4 (BioLegend) was used to stain individual donor CAR T cells for each condition. After 30 minutes incubation at 4° C., cells were washed twice with 1 mL of PBS 1× and finally resuspended in PBS at 1×10⁶ cells/ml for the downstream loading. For each condition, the 4 individual donors were pooled together at equal proportion a final sample for CITEseq submission. Final concentration for submission was ~1000 cells/μL, minimum 50 μL sample.

Cells were stained with Trypan blue and a Countess II Automated Cell Counter (ThermoFisher) was used to assess both cell number and viability. Following QC, the single cell suspension was loaded onto Chromium Next GEM Chip K (10× Genomics PN 1000286) for gel beads in emulsion (GEM) generation, cDNA synthesis, cDNA amplification, and library preparation of 10,000 cells proceeded using the Chromium Next GEM Single Cell 5′ Kit v2 (10× Genomics PN 1000263) according to the manufacturer's protocol. cDNA amplification included 13 cycles and 50 ng of the material was used to prepare sequencing libraries with 14 cycles of polymerase chain reaction (PCR). Indexed libraries were pooled equimolar and sequenced on a NovaSeq 6000 in a PE28/88 run using the NovaSeq 6000 S4 Reagent Kit (200 cycles) (Illumina). An average of 22,500 reads was generated per cell. Amplification products generated using the methods described above included both cDNA and feature barcodes tagged with cell barcodes and unique molecular identifiers. Smaller feature barcode fragments were separated from longer amplified cDNA using a 0.6× cleanup using aMPure XP beads (Beckman Coulter catalog #A63882). Libraries were constructed using the 5′ Feature Barcode Kit (10× Genomics PN 1000256) according to the manufacturer's protocol with 8 cycles of PCR. Indexed libraries were pooled equimolar and sequenced on a NovaSeq 6000 in a PE28/88 run using the NovaSeq 6000 S4 Reagent Kit (200 cycles) (Illumina). An average of 82 million paired reads was generated per sample.

### CITE-seq Analysis

FASTQ sequencing reads were processed using the Cell Ranger pipeline, which extracts cell barcodes, unique molecular identifiers (UMI), and cDNA reads or antibody barcodes, aligns cDNA reads to the human GRCh38 reference genome and generates gene and antibody UMI count matrices. Resultant-filtered sparse count matrices were loaded into R as a Seurat object. HTODemux was performed to assign cell barcodes to specific patient samples and droplet types (Stoeckius et al., Genome Biol. (2018); 19 (1): 224), and ambiguously assigned cells (i.e. doublet, unmapped, or negative) were removed.

Cells that passed the following QC filters were included in downstream analysis: (1) singlets identified by cell hashing, (2) cells with >200 and <5000 detected genes (outliers may represent empty droplets, low-quality cells, doublets, or multiplets), (3) cells with <20,000 cDNA UMI and <4000 ADT UMI (outliers may represent doublets or multiplets or cells with aberrant clumps of antibodies), and 4) cells with <5% mitochondrial gene expression (extensive mitochondrial contamination was often found in low quality or dying cells). In total, 28,855 cells across 16 samples (4 conditions with 4 biological replicates each) passed QC and were included in downstream analysis.

The RNA data was normalized using SCTransform (Hafemeister et al., Genome Biol. (2019); 20 (1): 296) and integrated across conditions using Seurat as previously described (Stuart et al., Cell (2019); 177 (7): 1888-902 e21). The ADT data was normalized using centered log-ratio transformation across cells. Dimensional reduction, identification of multimodal neighbors, UMAP visualization based on a weighted combination of RNA and ADT data, and graph-based clustering were performed using Seurat's weighted nearest neighbor workflow (Hao et al., Cell (2021); 184 (13): 3573-87 e29). The clusters were annotated based on conserved gene and surface protein markers identified across conditions using the FindConservedMarkers function as well as previously known markers of T cell type, proliferation, activation, cytotoxicity, cytokines, and dysfunction.

Differential gene and surface protein expression analyses between subsets of interest were performed using the Seurat's FindMarkers function which utilizes Wilcoxon rank-sum test. Gene set enrichment analysis against (GO) terms was performed using the fgsea package. Density plots grouped by gene sets were generated by using the frequency of fold change values per gene within each set. Additional functional analyses of differentially expressed genes were performed using the Human Molecular Signatures Database (MSigDB) hallmark gene sets from 2020 in the EnrichR package. All significant genes (adjusted P-value<0.05) were further evaluated using the transcription factor enrichment analysis tool ChIP-X Enrichment Analysis 3 (ChEA3) (Keenan et al., Nucleic Acids Res. (2019); 47 (W1): W212-W24).

The expression of a custom gene set associated with cytotoxicity (GZMA, GZMB, GZMH, GZMM, GZMK, NKG7, GNLY, PRF1) was scored in single cells using AddModuleScore, which calculates the average expression levels of all the genes in a given gene set and then subtracts the average expression levels of control gene sets (Tirosh et al., Science (2016); 352 (6282): 189-96). All genes were binned based on their average expression, and 5 control genes were randomly selected from each bin. This method controls expression of the gene set for differences in cell quality and library complexity across single cells. The Seurat's Featureplot function was used to visualize expression of this cytotoxicity gene set across clusters on the multimodal UMAP.

### Establishment of Patient-Derived Tumor Xenografts and Derivation of PDX Cells

Fresh tumor specimens were multiregionally sampled from liver metastases of pancreatic cancer autopsy, as approved by our institutional review board-approved protocols (#15-149, and #15-021). Tissues were immediately rinsed with sterile saline, chopped into 2-4 mm pieces and immediately transplanted subcutaneously in the flanks of NSG mice and followed for patient derived xenograft (PDX) development. Primary metastases tumor as well as the PDX tumors were also chopped into 1-2 mm pieces with collagenase Type IV (Stem Cell Technologies/0.5 mg/mL) for 1 hr at room temperature. The cells were sieved through 100-micron (fisher Brand) nylon mesh and collected by centrifugation. The cells were washed twice in serum free RPMI medium and were immediately injected (with 1:1 dilution in Matrigel) into the NSG mice flanks subcutaneously or orthotopically into the pancreas under our IACUC approved protocol (14-02-002). A portion of the cells was also plated on tissue culture coated plates to passage the primary cells for 2-4 passages.

In brief, an injection of 2 mg/kg of meloxicam was given subcutaneously for pre-emptive analgesia immediately after the animal was anesthetized. A small volume (0.1 cc) of local anesthetic agent, such as bupivacaine (Marcaine 0.25%), was infiltrated into the tissue adjacent to the intended incision line. The skin was then painted with a 10% povidone-iodine (Betadine®) or chlorhexidine (Nolvasan®) solution. Sterile scissors were then used to make a 0.5-1 cm parasagittal incision through the abdominal musculature over the spleen. The injection was made into the pancreatic parenchyma below the capsule using a 28G needle injecting a maximum volume of 50 μl. The spleen and pancreas were gently replaced within the peritoneal cavity. The muscle layer was closed using sterile absorbal suture (e.g., Vicryl) of the appropriate diameter in a simple interrupted pattern. Skin edges were closed with sterilized wound clips (Autoclips) or with a monofilament absorbable suture (e.g. Monocryl) of the appropriate diameter in a subcuticular pattern.

### Necropsy and Histopathology

Mice were euthanized with CO₂. Following gross examination all organs were fixed in 10% neutral buffered formalin, followed by decalcification of bone in a formic acid solution (Surgipath Decalcifier I, Leica Biosystems). Tissues were then processed in ethanol and xylene and embedded in paraffin in a Leica ASP6025 tissue processor. Paraffin blocks were sectioned at 5 microns, stained with hematoxylin and eosin (H&E), and examined by a board-certified veterinary pathologist. The following tissues were processed and examined: heart, thymus, lungs, liver, gallbladder, kidneys, pancreas, stomach, duodenum, jejunum, ileum, cecum, colon, lymph nodes (submandibular, mesenteric), salivary glands, skin (trunk and head), urinary bladder, uterus, cervix, vagina, ovaries, oviducts, adrenal glands, spleen, thyroid gland, esophagus, trachea, spinal cord, vertebrae, sternum, femur, tibia, stifle join, skeletal muscle, nerves, skull, nasal cavity, oral cavity, teeth, ears, eyes, pituitary gland, brain.

### Hematologic Assays

For hematology, blood was collected into tubes containing EDTA (ethylenediaminetetraacetic acid). Automated analysis was performed on an IDEXX Procyte DX hematology analyzer and the following parameters were determined: white blood cell count, red blood cell count, hemoglobin concentration, hematocrit, mean corpuscular volume, mean corpuscular hemoglobin, mean corpuscular hemoglobin concentration, red blood cell distribution width standard deviation and coefficient of variance, reticulocyte relative and absolute counts, platelet count, platelet distribution width, mean platelet volume, and relative and absolute counts of neutrophils, lymphocytes, monocytes, eosinophils, and basophils.

### Serum Chemistry Assays

For serum chemistry, blood was collected into tubes containing a serum separator, the tubed were centrifuged and the serum was obtained for analysis. Serum chemistry was performed on a Beckman Coulter AU680 analyzer, and the concentration of the following analytes was determined: alkaline phosphatase, alanine aminotransferase, aspartate aminotransferase, creatine kinase, gamma-glutamyl transpeptidase, albumin, total protein, globulin, total bilirubin, blood urea nitrogen, creatinine, cholesterol, triglycerides, glucose, calcium, phosphorus, chloride, potassium, and sodium. Sodium/potassium ratio, albumin/globulin ratio were calculated.

### Quantification and Statistical Analysis

All statistical analyses were performed using GraphPad Prism software, RRID: SCR_002798 (GraphPad). Data points represent biological replicates and are shown as the mean±SEM or mean±SD as indicated in the figure legends. Statistical significance was determined by paired two-tailed Student's t-test, one-way ANOVA, or two-way ANOVA as indicated in the figure legends. The log-rank (Mantel-Cox) test was used to determine statistical significance for overall survival in mouse survival experiments. Significance was indicated with *, P<0.05; **, P<0.01; ***, P<0.001; and ****, P<0.0001.

### Results

### LTβR is Necessary for LIGHT-Mediated Tumor Cytotoxicity

A γ-retroviral vector was used to engineer healthy donor-derived human T cells to express both the CAR and LIGHT. A previously described CD371-directed single chain variable fragment (scFV-B10H4L clone, No. PCT/US2020/050386) was fused to a myc tag to allow for CAR detection, and inserted upstream of human CD28, and CD3ζ signaling domains to generate a human second-generation CAR (CD371-28z). To generate the corresponding LIGHT-modified CAR T cells, the CD371-28z construct was inserted upstream of a P2A self-cleaving element, which was itself upstream of a human LIGHT transgene (CD371-28z-LT). A negative control, non-functional CAR T cell lacking the intracellular signaling portion of the CAR and unable to elicit downstream function upon antigen recognition was also generated (CD371-Del; FIG. 3A).

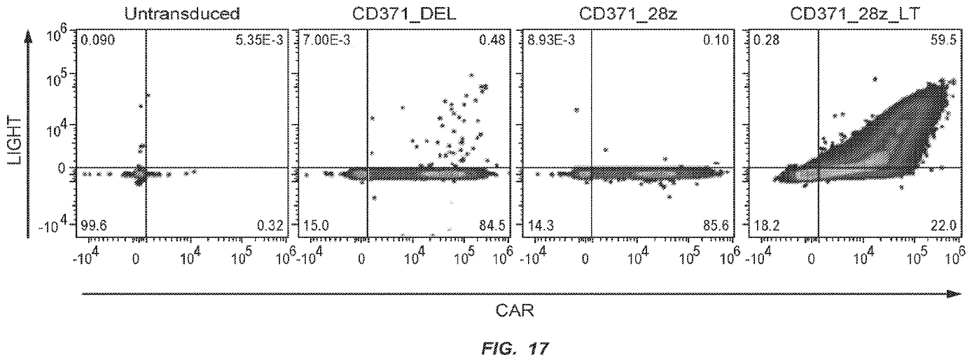

Expression of engineered CAR and LIGHT constructs in primary human T cells was validated (FIG. 17). LIGHT-CAR T cells were then evaluated for cytotoxicity against acute myeloid leukemai (AML) tumor cells, which endogenously express the AML-associated antigen CD371. In co-culture assays of firefly luciferase-expressing (Luc+) tumor cells with CAR T cells, CD371-directed CAR T cells specifically killed CD371+U937 AML cell lines as expected (FIG. 6A), and second-generation CAR T cells were as cytotoxic as LIGHT-CAR T cells against AML. In vitro cytolysis was measured by bioluminescence.

To evaluate any additive cytolysis mediated through LIGHT, which may have been concealed by CAR-mediated killing, LIGHT-CAR T cells were tested against a CD371 knockout (KO) U937 AML cell line (FIG. 6B), and against CD371-low/negative AML cell lines (FIGS. 6C and 6D. Interestingly, LIGHT-CAR T cells outperformed second-generation CD371-CAR T cells in killing CD371-low and negative AML cell lines. To assess whether this enhanced cytotoxicity was mediated by LIGHT, CRISPR was used to generate knockout cell lines that lacked expression of the known receptors for LIGHT, (LTβR and HVEM), in the CD371KO U937 AML cell line (FIGS. 7A-7D). Co-culture assays of these cells with the second-generation CAR T cells and LIGHT-CAR T cells demonstrated that removal of LTβR from tumor cells conferred resistance to the effects of LIGHT, whereas removal of HVEM did not (FIGS. 8A-8D). These data suggested that LIGHT-CAR T cells killed tumor cells in an LTβR dependent manner through an unknown mechanism.

Cytotoxicity of CD371-LIGHT CAR T cells was evaluated against various CD371-negative solid tumor cell lines expressing LTβR (A375 melanoma, HT-29 colorectal adenocarcinoma, and PATU8988t pancreatic adenocarcinoma). Consistent with previous findings in AML, CD371-LIGHT CAR T cells were more effective than second-generation CAR T cells in suppressing the outgrowth of these cell lines (FIGS. 6E-6G).

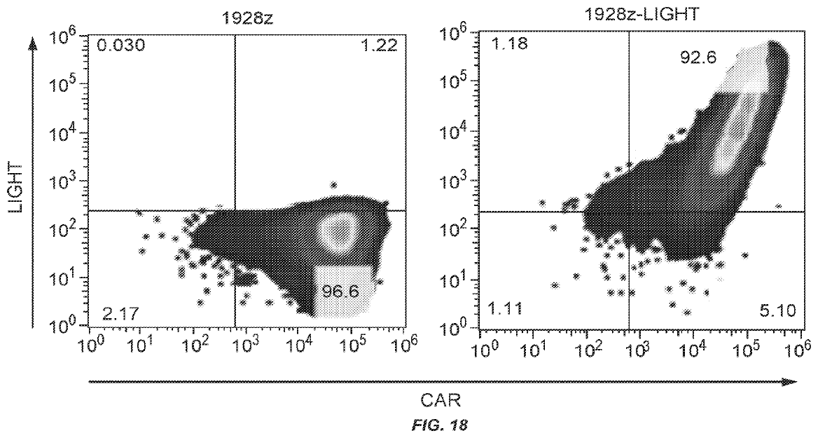

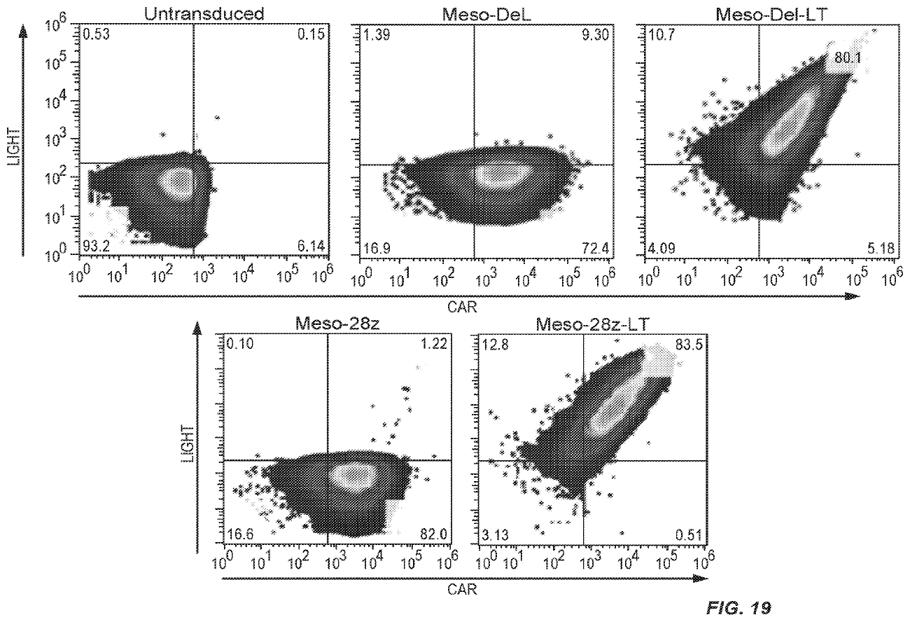

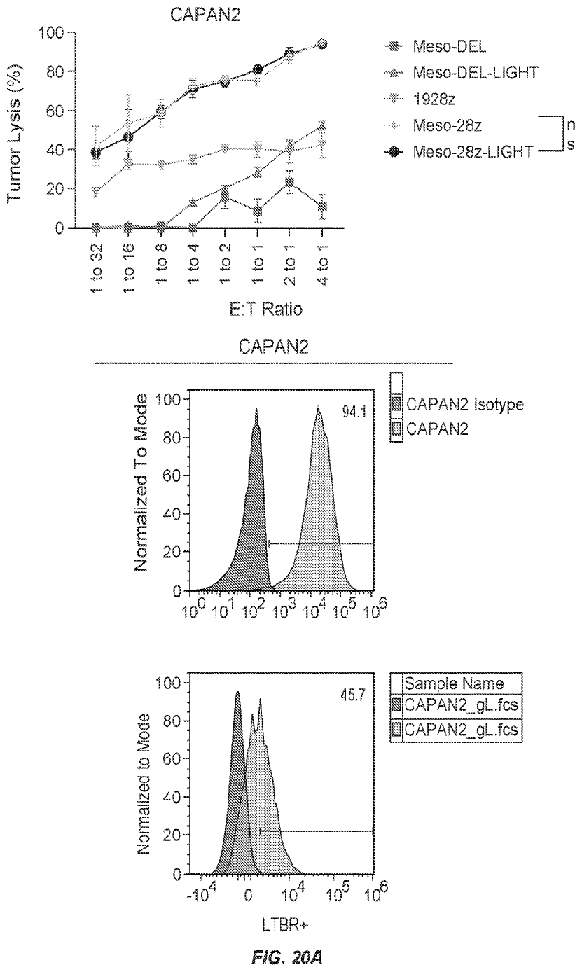

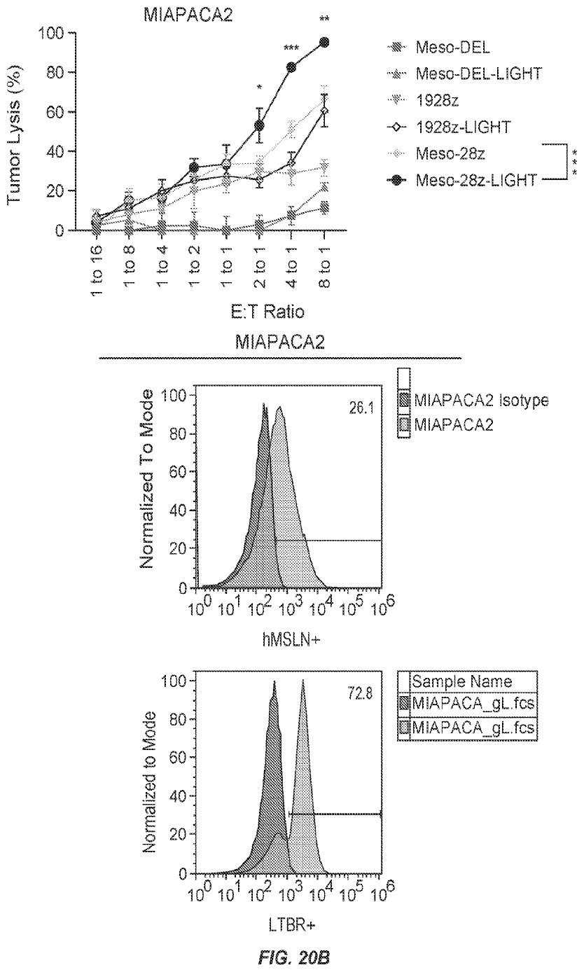

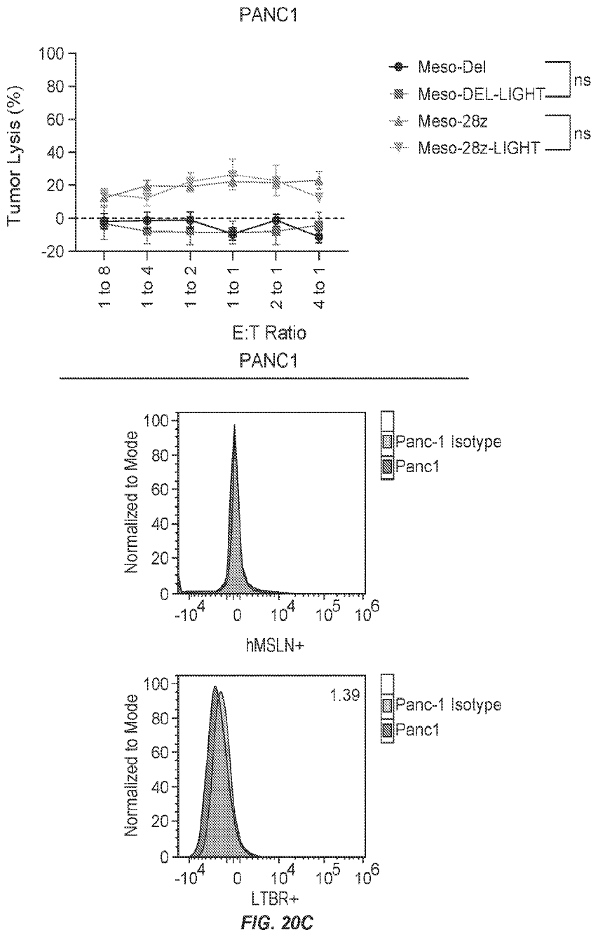

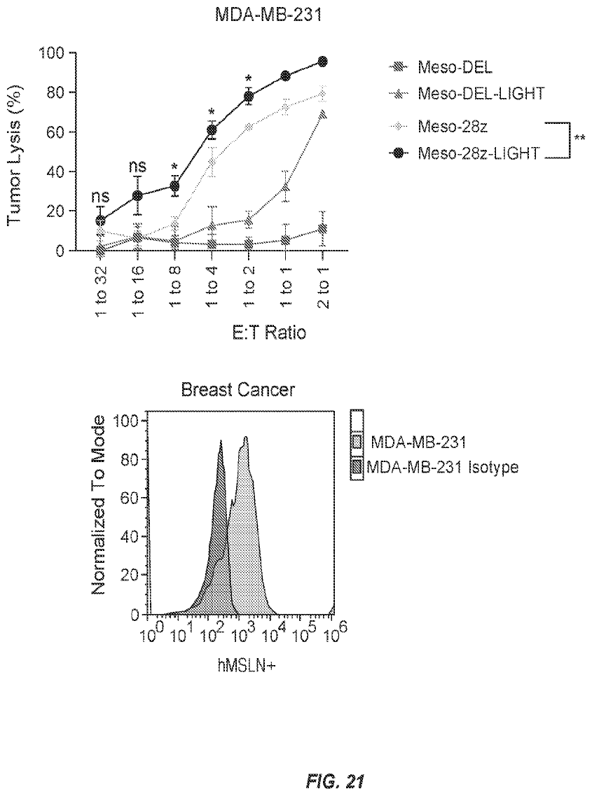

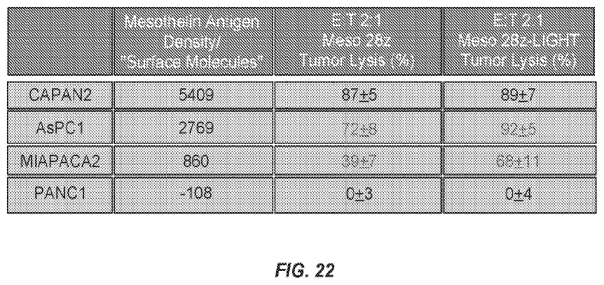

To determine whether another feature of CD371-CAR T cells explains their augmented activity, another CAR T cell construct was developed to be directed against mesothelin, a widely overexpressed tumor-associated antigen found on a variety of cancers (Morello et al., Cancer Discov (2016); 6 (2): 133-46; Adusumilli et al., Sci Transl Med. (2014); 6 (261): 261ra151). An additional construct, termed Meso-Del-LT, lacked the intracellular signaling portion of the CAR but retained the LIGHT transgene (FIG. 3B). CD19-directed CAR T constructs, 1928z and 1928z-LIGHT (FIG. 18) were also designed as irrelevant antigen targeting CAR T cell controls. The transgene expression of both the mesothelin-directed CAR and LIGHT in primary human T cells was confirmed by flow cytometry (FIG. 19). As pancreatic ductal adenocarcinoma (PDAC) was composed of heterogeneous populations of mesothelin positive cells, multiple PDAC cell lines with varied mesothelin expression were used and their sensitivity to CAR and LIGHT-modified CAR T cell-mediated tumor lysis were assessed via in vitro cytotoxicity assay. Consistent with the previous findings, second-generation CAR T cells and LIGHT-CAR T cells had similar cytotoxic effects in a cell line with high target-antigen density (FIG. 20A), but LIGHT augments CAR T cells outperformed second-generation CAR T cells against cells with low target-antigen density (FIG. 11D and FIG. 20B). The mesothelin-directed CAR T cells against a variety of mesothelin-positive cell lines were tested and similar results were observed with LIGHT-CAR T cells displaying increased cytotoxicity as compared to the corresponding second-generation CAR T cells (FIGS. 11G and 11I, FIG. 21). Panc1 was used as a negative control and displayed no killing without expression of mesothelin and LTβR (FIG. 20C). When comparing the cytotoxicity of LIGHT-CAR T cells to second-generation CAR T cells against different PDAC cell lines, it was observed that LIGHT-CAR T cells exhibited superior cell-killing activity when the target CAR-antigen was not present in high abundance (FIG. 22).

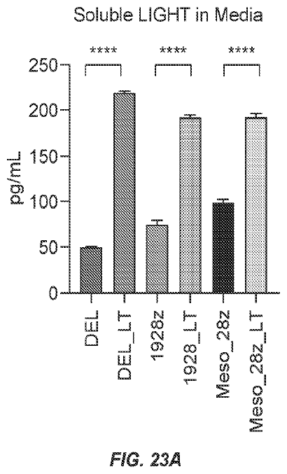

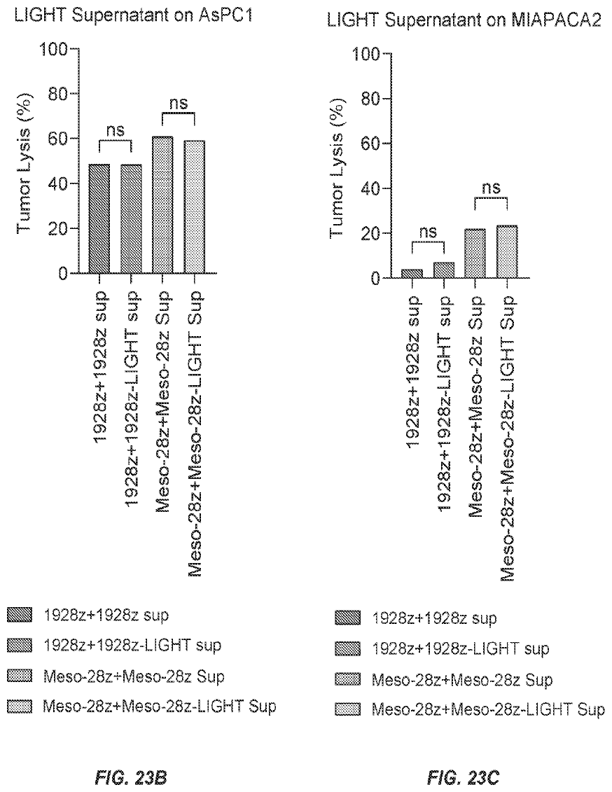

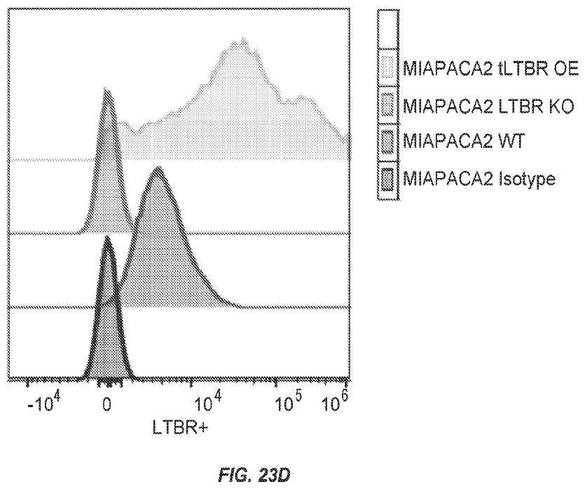

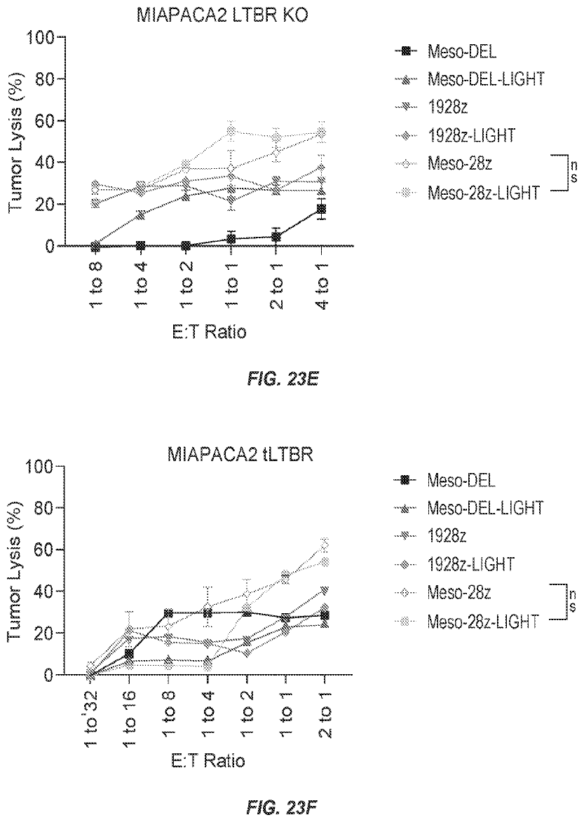

It was also investigated whether LIGHT in its soluble form (sLIGHT) would confer similar effects on CAR T cells as in its membrane-bound isoform. Briefly, supernatant containing sLIGHT secreted from LIGHT-CAR T cells was collected, quantified via Enzyme-Linked Immunosorbent Assay (ELISA; FIG. 23A), and added to second-generation 1928z or meso-28z CAR T cells when a cytotoxicity assay against AsPC1 and MIAPACA2 was performed. Supernatant derived from LIGHT-CAR T cells did not enhance the cytotoxicity of second-generation CAR T cells (FIGS. 23B and 23C).

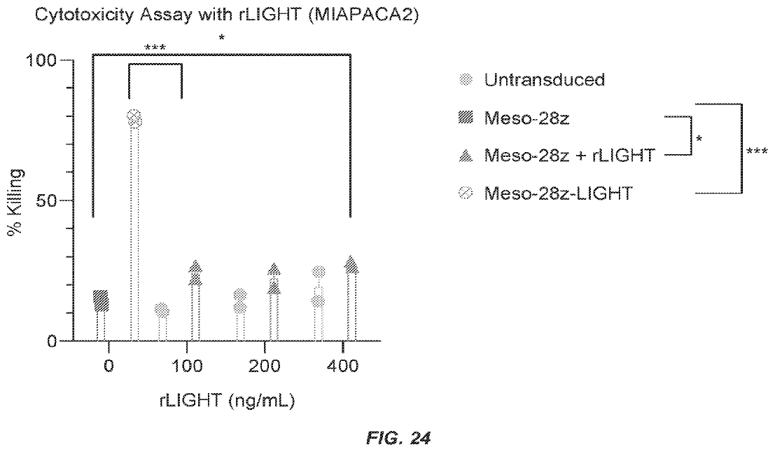

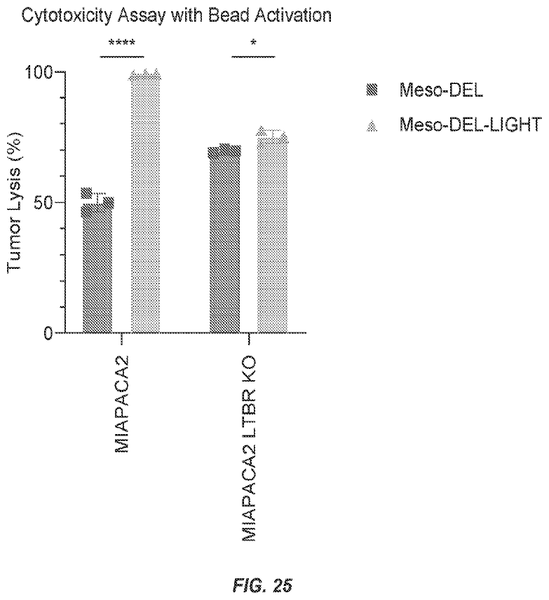

In case the amount of sLIGHT produced by LIGHT-CAR T cells was insufficient to confer any effect, a concentration of recombinant LIGHT (rLIGHT) previously reported to have a biological effect was also used. This resulted in a slight increase in the cytotoxicity of second-generation CAR T cells against MIAPACA2 that was still much lower than that of LIGHT-CAR T cells (FIG. 24), suggesting that sLIGHT was not the primary mediator of augmented cytotoxicity. It was then tested whether a non-functional CAR with LIGHT (Meso-DEL-LIGHT) would kill cancer cells in the presence of exogenous CD3/28 stimulation via antibody-coated beads (Dynabeads). Interestingly, the CD3/CD28 stimulation enhanced Meso-DEL-LIGHT CAR T cells cytotoxicity against MIAPACA2 compared to meso-DEL CAR T cells. Additionally, when repeated with an LTβR KO of the same cancer cell line, the augmented cytotoxicity was abolished, further indicating that the cytotoxicity was driven by LIGHT-LTβR engagement (FIG. 25).

### LIGHT Confers Improved Co-Stimulation and a Proliferative Advantage to T Cells

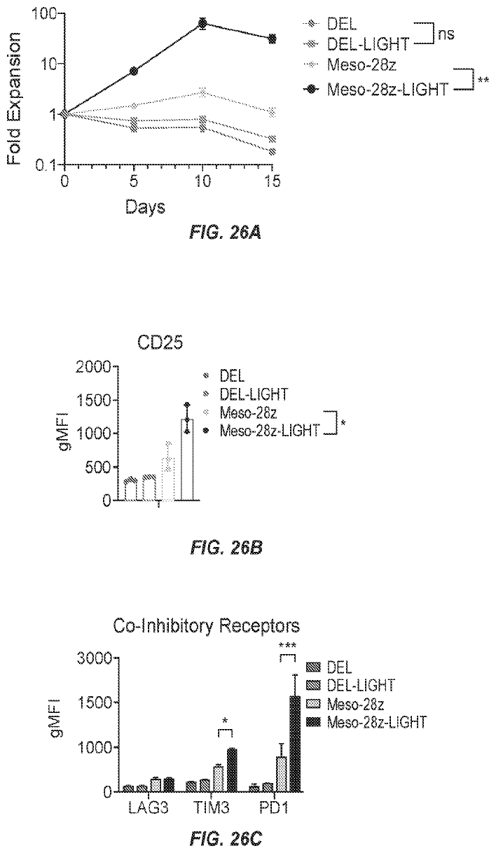

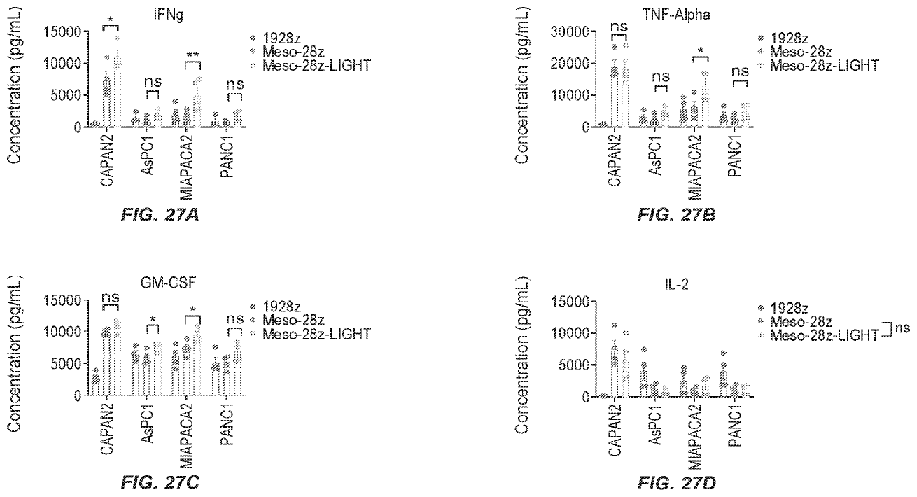

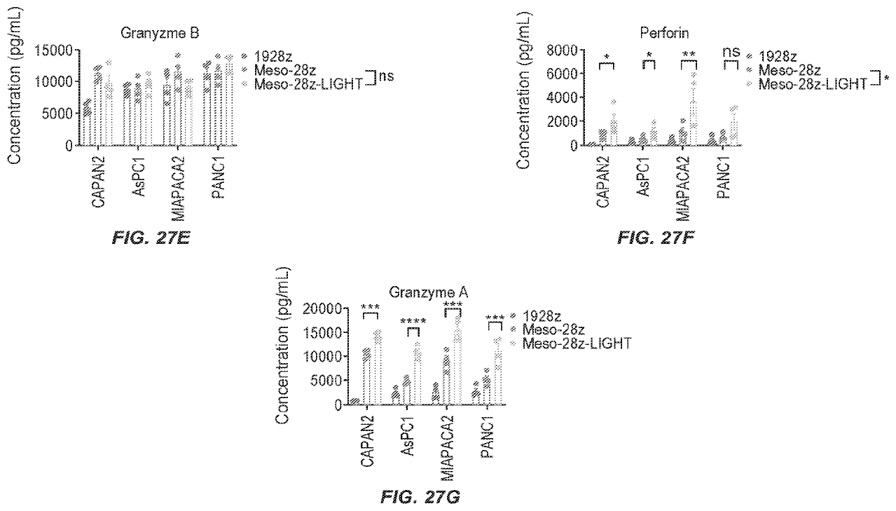

It was investigate whether LIGHT provided additional costimulation to CAR T cells by interrogating its effect on the activation, proliferation, and persistence of CAR T cells. In the resting state, there were no noticeable differences in the proliferation profile of second-generation and LIGHT-CAR T cells. However, when these cells were co-cultured with the PDAC cell line, MIAPACA2, LIGHT-CAR T cells exhibited better proliferation and persistence in a repetitive antigen stimulation assay (FIG. 26A). On day 15, these CAR T cells were evaluated for activation marker IL2RA (CD25) and co-inhibitory receptors (PD-1, LAG-3, and TIM-3) by flow cytometry (FIGS. 26B and 26C). Secretion of proinflammatory cytokines (e.g., IFNγ, perforin, GM-CSF, and in some cases TNF-alpha) were significantly higher in LIGHT-CAR T cells, suggesting that cytokine levels contributed to their superior cytotoxicity (FIGS. 27A to 27G). Interestingly, no significant increase in IL-2 or granzyme (GZM) B secretion was observed. GZMA was significantly higher in LIGHT-CAR Tcells (FIG. 27G).

Single-Cell Multiomic Profiling of LIGHT-CAR T Cells Reveals More Activated Cell States With Higher Expression of Cytotoxicity Genes and Cytokines/Chemokines Upon Co-Culture with Cancer Cells

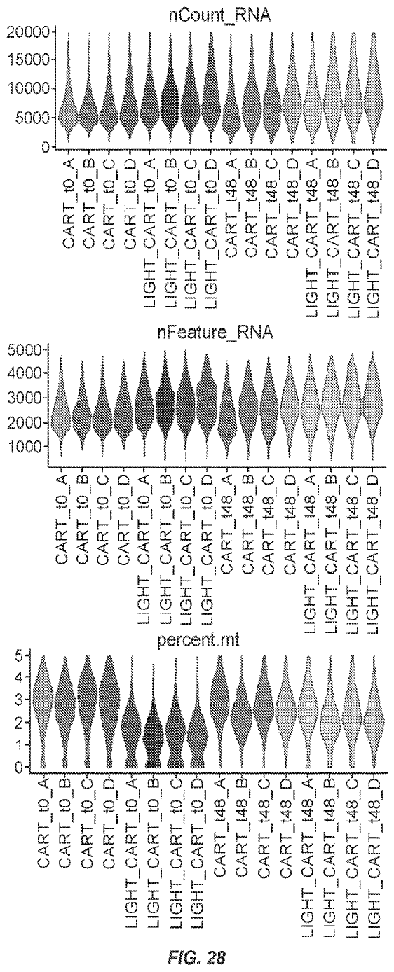

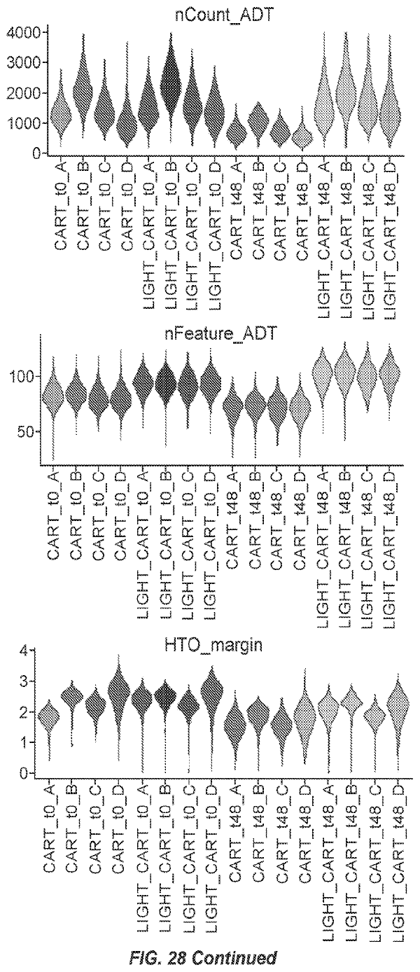

Cellular indexing of transcriptomes and epitopes by sequencing (CITE-seq) were performed to evaluate the transcriptional state and surface protein expression of individual CAR T cells before and after co-culture with cancer cells. Mesothelin-directed CAR T cells with and without LIGHT (Meso-28z-LT and Meso-28z, respectively) were generated from 4 healthy human donors and sequenced post-sorting at baseline (t=0) and 48 hours after co-cultured with cancer cells (t=48h). Cell hashing (Stoeckius et al., Genome Biol. (2018); 19 (1): 224) was performed to combine samples of the same condition (construct and timepoint) from different donors and a panel of 130 antibody-derived tags (ADTs) was used to stain cell surface proteins. A total of 28,855 cells was obtained across 16 samples that passed quality control (QC) for downstream analysis (FIG. 28).

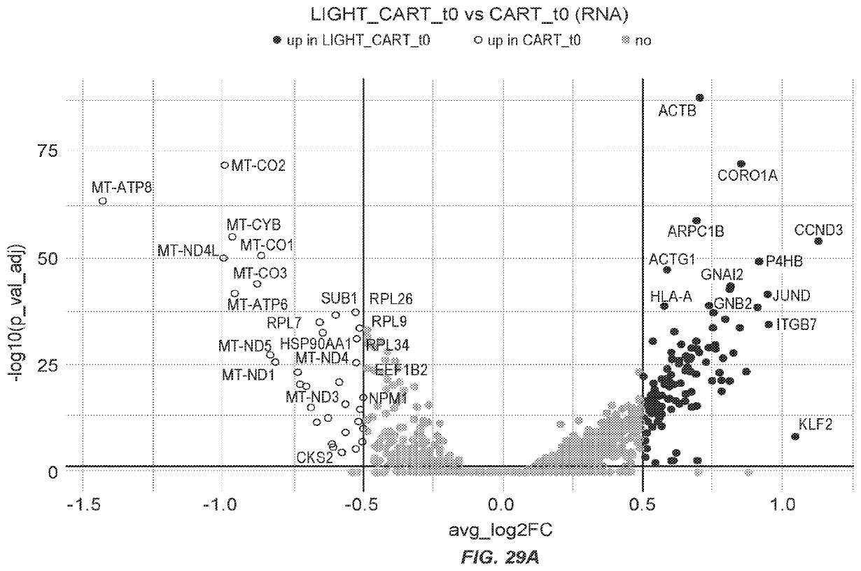

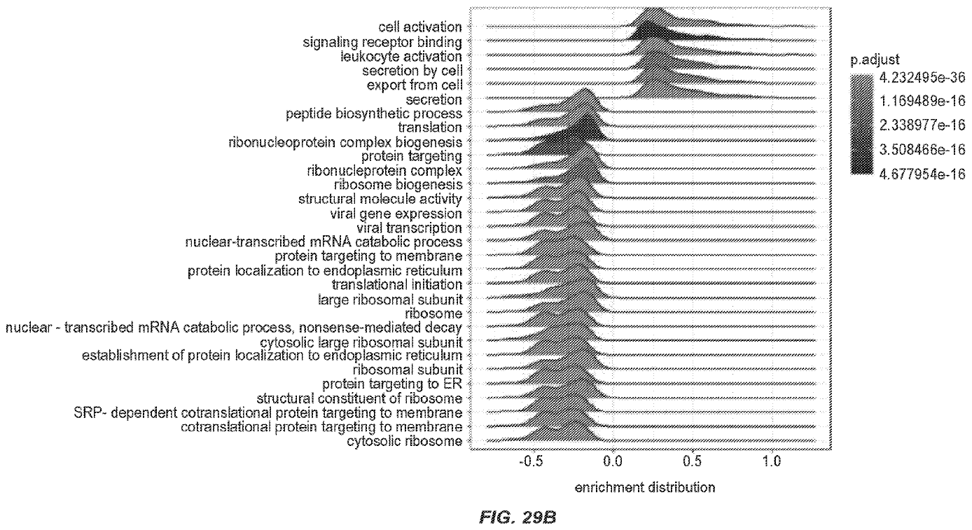

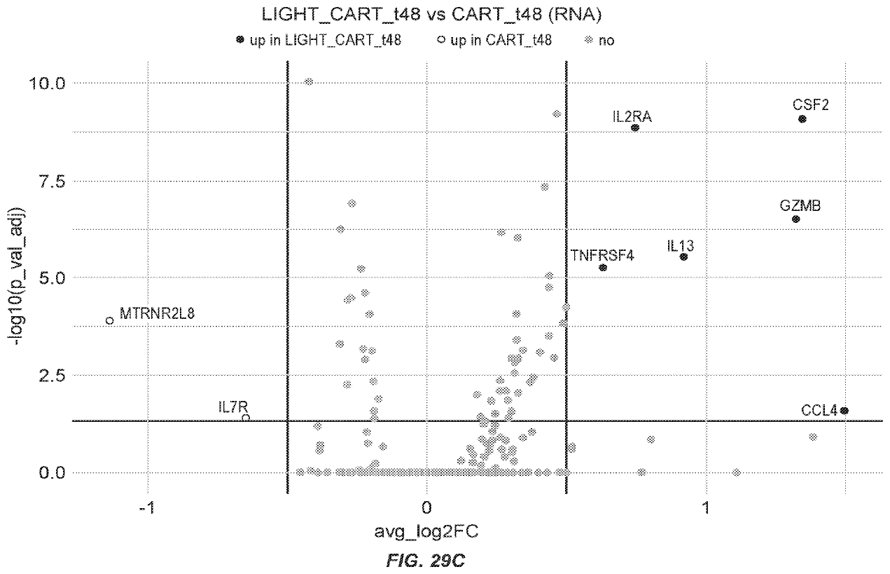

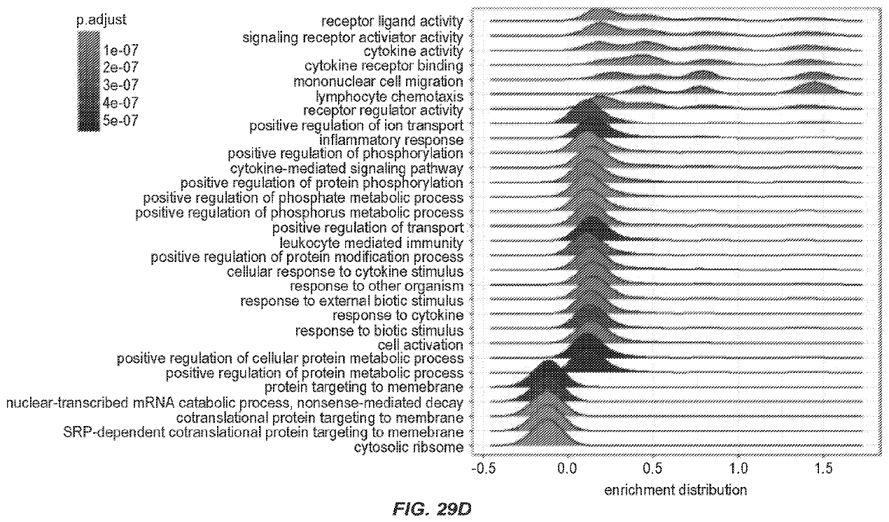

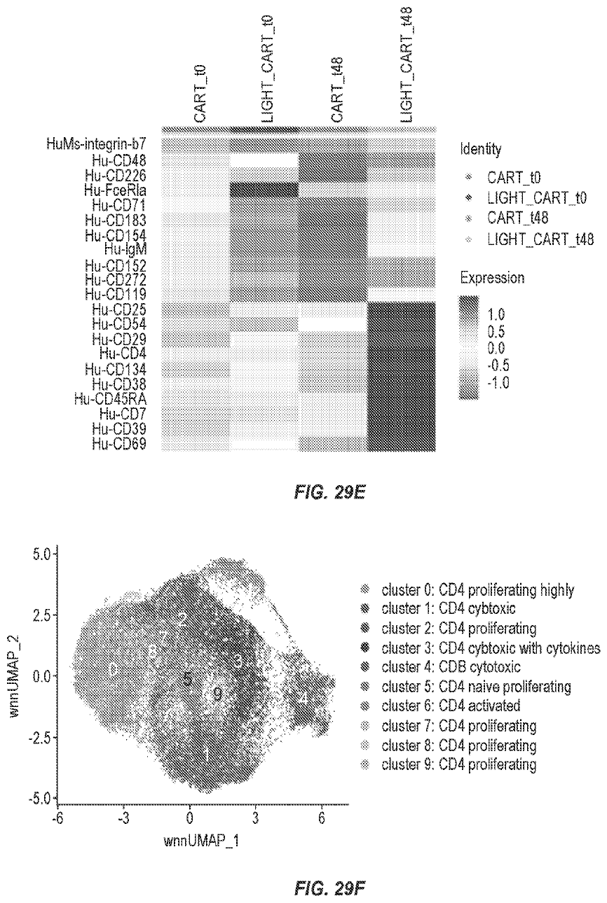

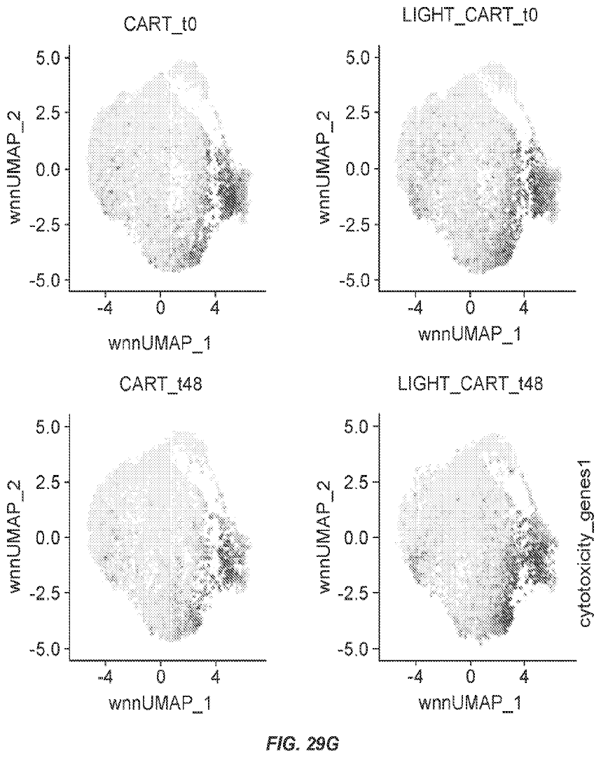

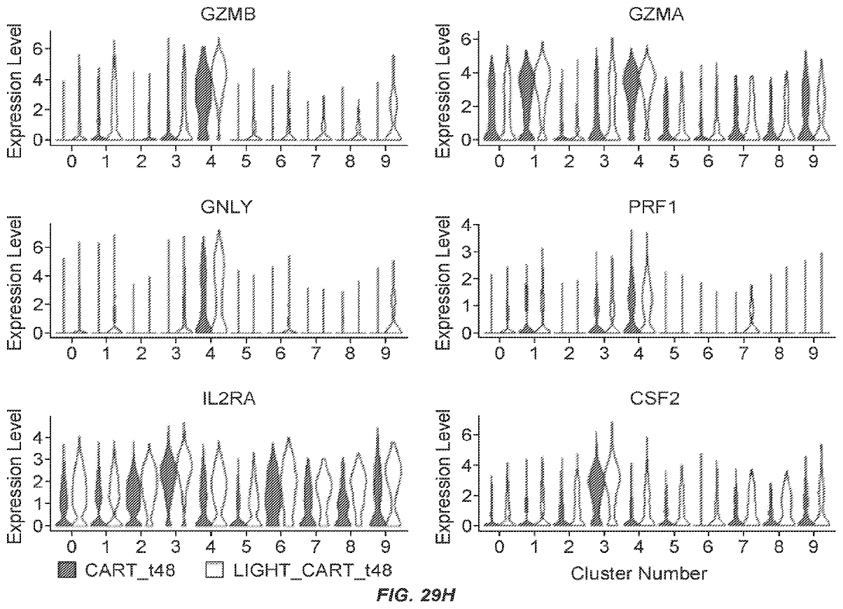

The transcriptional profiles of Meso-28z and Meso-28z-LT cells differed at baseline. LIGHT-CAR T cells expressed significantly higher levels of ACTB, CCND3, JUND, ITGB7, KLF2, and HLA-A, among many other genes (FIG. 29A), and their profiles were enriched for gene ontology (GO) terms such as T cell activation, signaling receptor binding, and cell secretion/export compared with controls (FIG. 29B). At t=48 h after co-culture, LIGHT-CAR T cells expressed higher levels of gene associated with cytotoxicity and inflammatory cytokines/chemokines, including CSF2 (Colony-stimulating factor 2/GM-CSF), GZMB, TNFRSF4 (OX40), IL2RA, CCL4, and IL13 (FIG. 29C). And their profiles were enriched for GO terms such as receptor-ligand activity, cytokine activity, and T cell migration/chemotaxis compared with controls CAR T cells (FIG. 29D).

LIGHT-CAR T cells also expressed higher levels of multiple cell surface proteins to control CAR T cells, including CD71 (a marker for T cell activation and proliferation), and CD272 (BTLA, a co-signaling molecule in the CD28 superfamily that binds to HVEM and influences the LIGHT-HVEM signaling), they expressed higher levels of additional activation markers, including IL25RA and CD69.

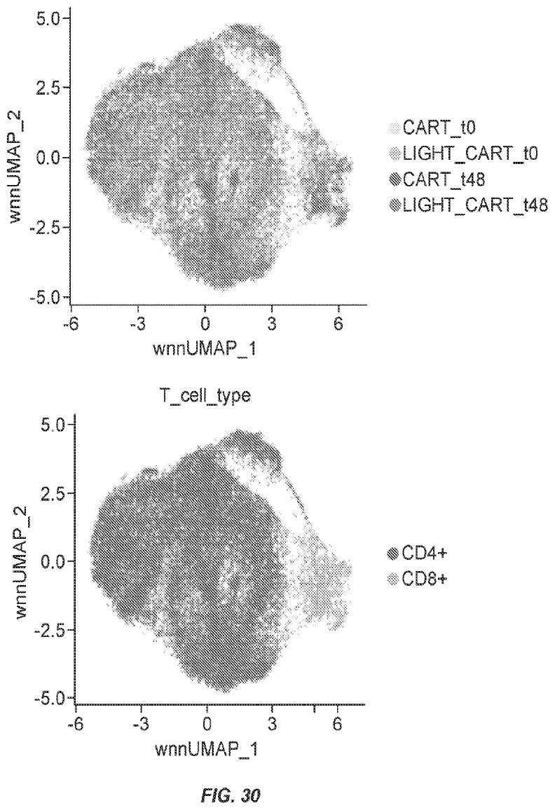

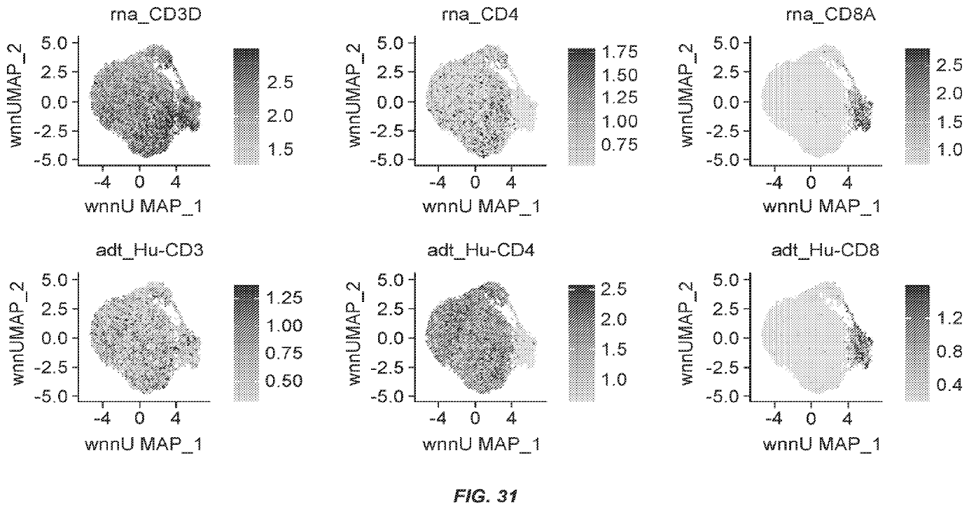

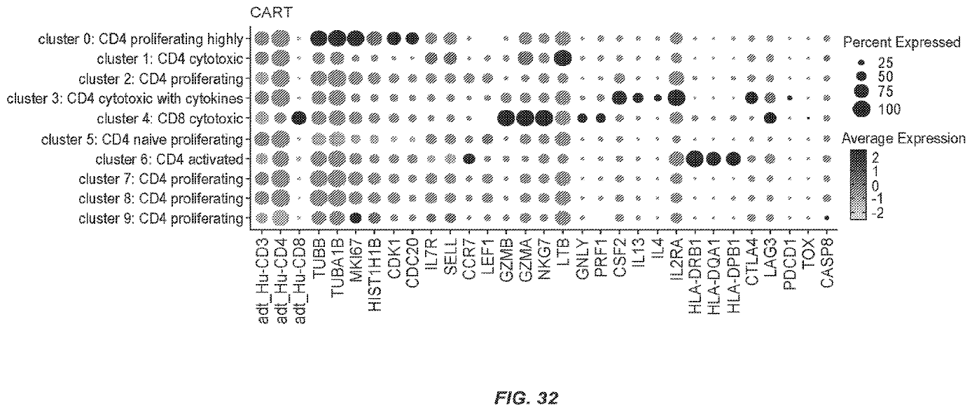

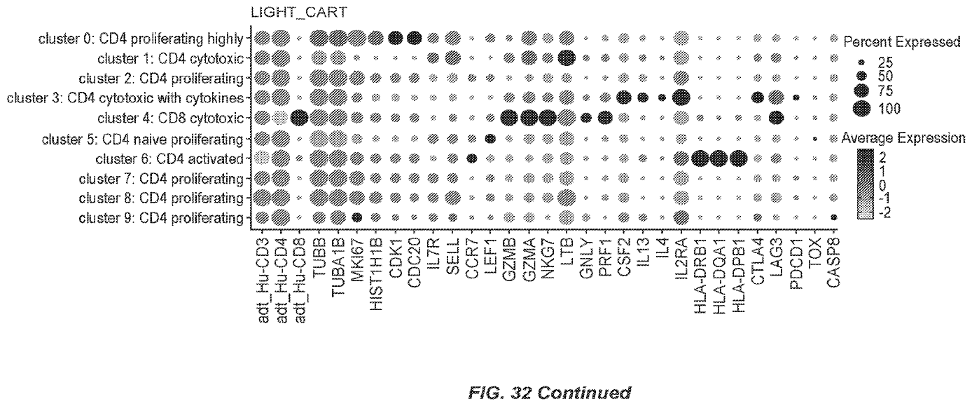

Features of enhanced cytotoxicity in subsets of LIGHT-CAR T cells was then investigated. After integrating our CITE-seq data and clustering single cells using a weighted combination of RNA and ADT, 10 clusters (shared cell states) across conditions were identified and visualized using a weighted-nearest neighbor (WNN) Uniform Manifold Approximation and Projection (UMAP) (FIG. 29F). Using conserved genes and surface proteins across conditions and previously known markers of T cell type, proliferation, activation, cytokines, and dysfunction, as well as cytotoxicity (GZMA, GZMB, GZMH, GZMM, GZMK, NKG7, GNLY, PRF1), 9 clusters of CD4⁺ CAR T cells with variable proliferative, cytotoxic, activated, and inflammatory features were annotated and 1 cluster of CD8⁺ CAR T cells with strong cytotoxicity features was annotated (FIG. 29F, FIG. 30, and FIG. 32). LIGHT-CART cells belonging to CD8⁺ cluster (cluster 4) and 2 CD4⁺ clusters (cluster 1 and 3) most highly expressed the predefined cytotoxicity genes at baseline and t=48 h compared with controls (FIG. 29G). Specifically, LIGHT-CAR T cells had greater upregulation of GZMB, GNLY, at t=48 h compared with controls, and this effect was most pronounced in clusters 1, 3, and 4 (FIG. 29H). LIGHT-CAR T cells also demonstrated greater expression of IL2RA and CSF2 (GM-CSF) at t=48h, but this effect was more generalized across clusters. Overall, these findings indicated that LIGHT-CAR T cells were more activated at baseline and after co-culture with cancer cells and exhibited features consistent with stronger cytotoxic and effector functions upon encounter with cancer cells-corroborating the in vitro data showing improved functionality of LIGHT-CAR T cells compared with standard CAR T cells.

### LIGHT-CAR T Cells Limit PDAC Tumor Outgrowth and Confer Survival Benefits in Xenograft Models

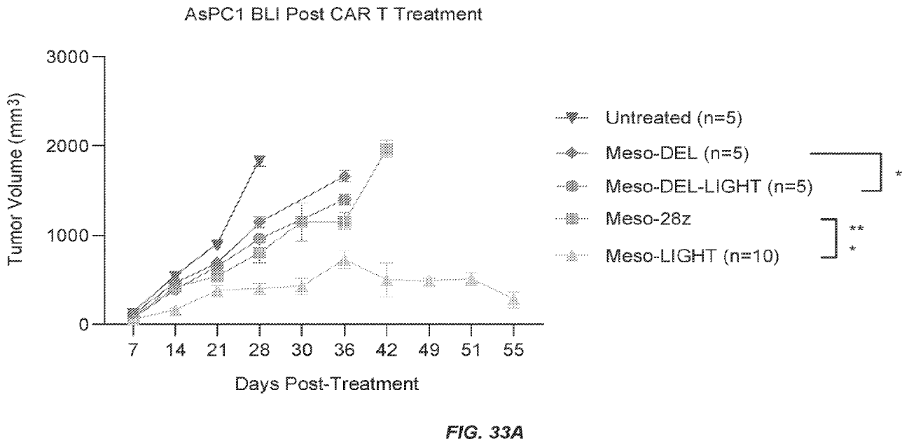

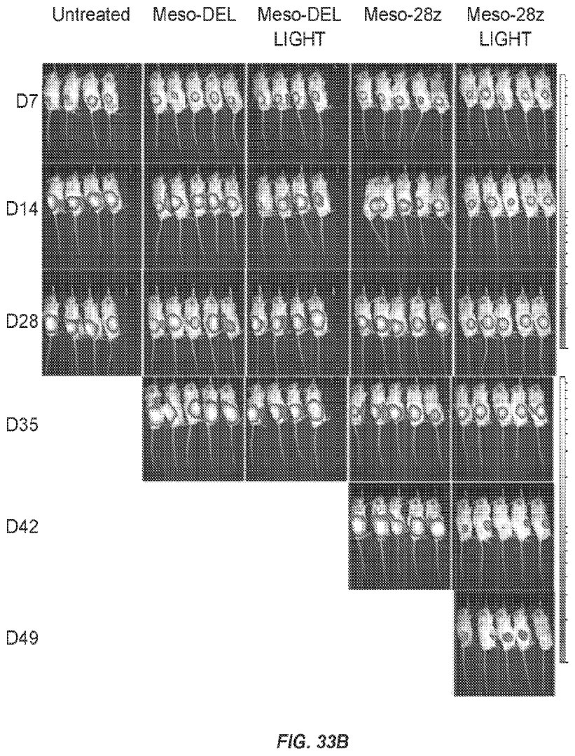

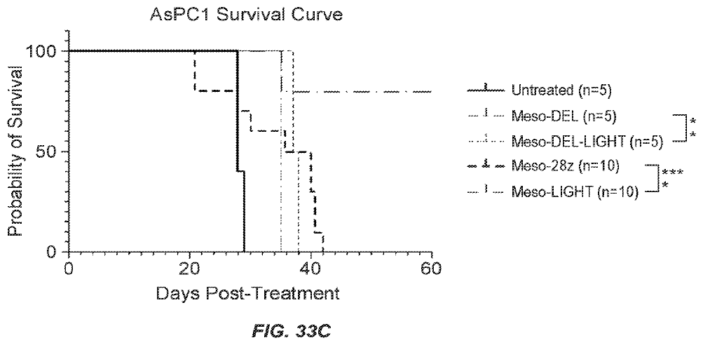

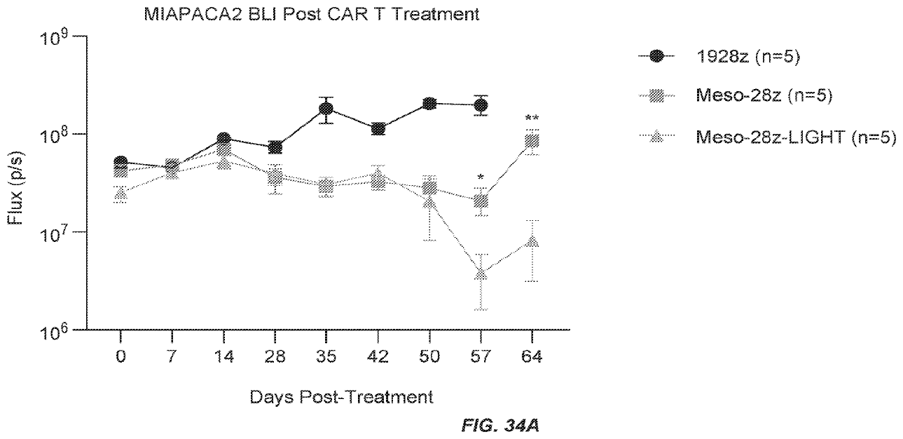

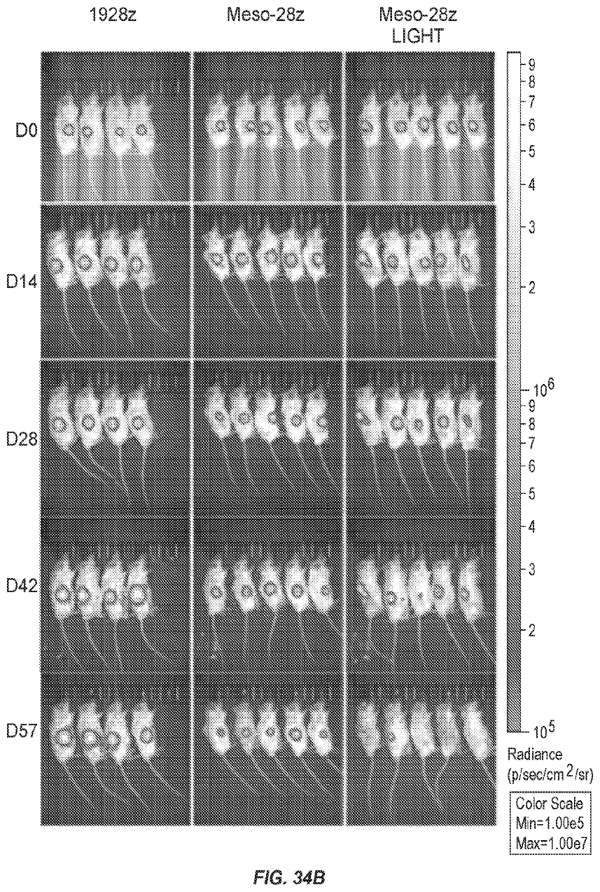

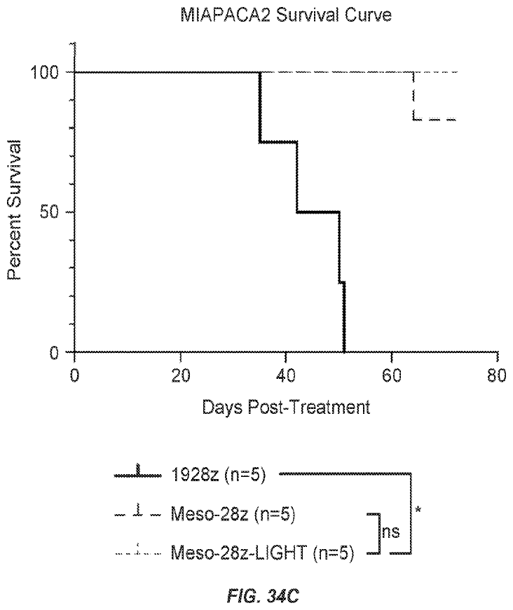

To evaluate the in vivo efficacy of mesothelin-directed LIGHT-CAR T cells, a xenograft flank model using AsPC1 PDAC cell line was established (FIG. 15A), which was chosen given the excellent expansion and cytotoxic effect of LGHT-CAR T cells in vitro. Caliper measurement and bioluminescent imaging (BLI) of tumor volume showed that LIGHT-CAR T cells controlled tumor outgrowth; the cells were also associated with significantly improved overall survival compared with the standard second-generation CAR T cells (FIGS. 33A to 33C). Given this success in tumor control, another PDAC cell line, (MIAPACA2) was tested, which expressed very low levels of mesothelin but exhibited susceptibility to LIGHT-mediated cytotoxicity, as described above (FIG. 20B). Consistent with the in vitro data, LIGHT-CAR T cells demonstrated superior tumor control on tumor BLI in vivo (FIG. 16A, FIG. 34A, and FIG. 34B). Despite this, the MIAPACA2 cell line was not very aggressive, and no mice were euthanized because of tumor volume or significant weight loss. Mice were euthanized on day 64, following the onset of graft-versus-host disease (GVHD; FIG. 34C).

### Evaluation of LIGHT-CAR T Cells in Orthotopic Patient-Derived Xenograft (PDX) Model of Human PDAC

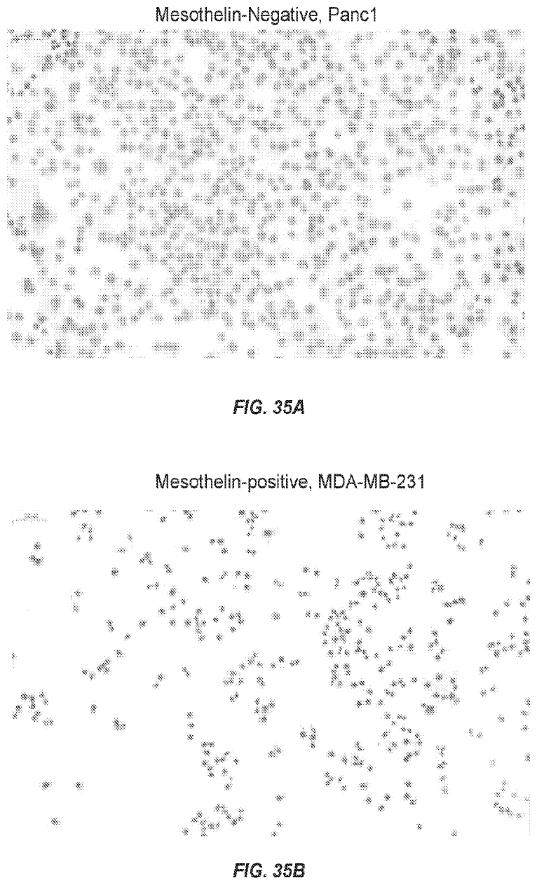

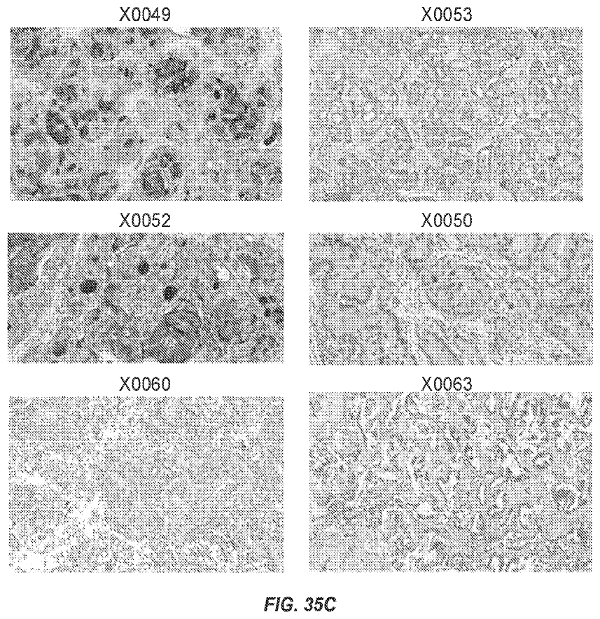

To evaluate the efficacy of LIGHT-CAR T cells in a more clinically relevant model, an orthotopic, patient-derived xenograft model of PDAC was used. Mesothelin expression was first screened using immunohistochemistry on a variety of PDX models (FIGS. 35A-35C). The surface expression of mesothelin was measured by flow cytometry; an in vitro cytotoxicity assay was performed; and the functional activity of the mesothelin-directed CAR T cells in a PDX sample was assessed (FIG. 11J). Consistent with our previous findings, LIGHT-CAR T cells demonstrated higher cytotoxicity compared with second-generation CAR T cells (FIG. 11J).

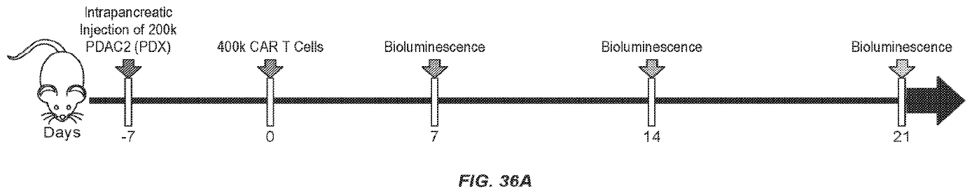

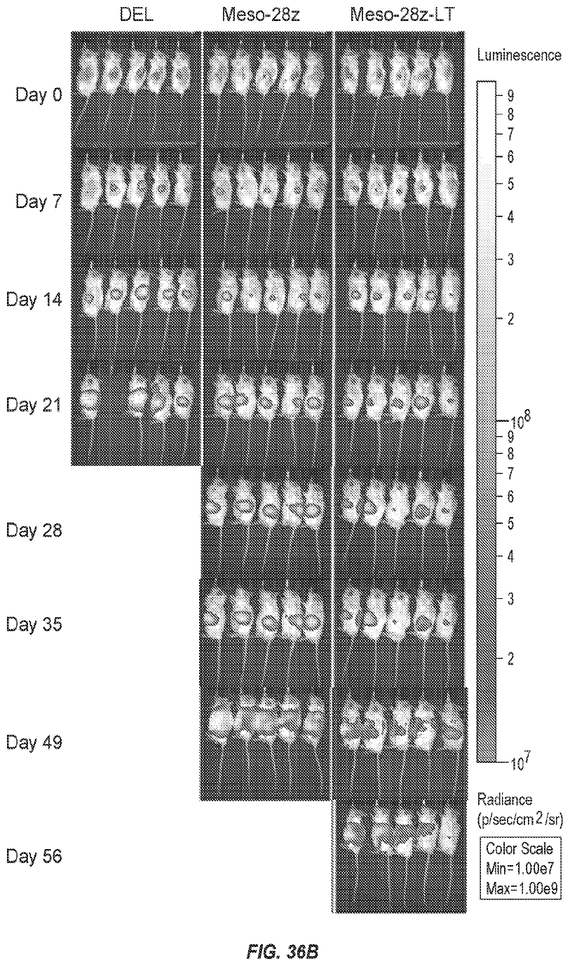

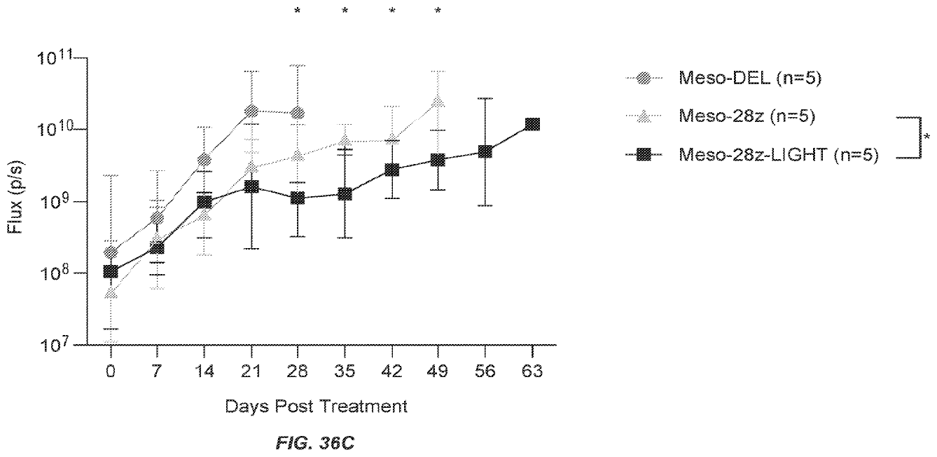

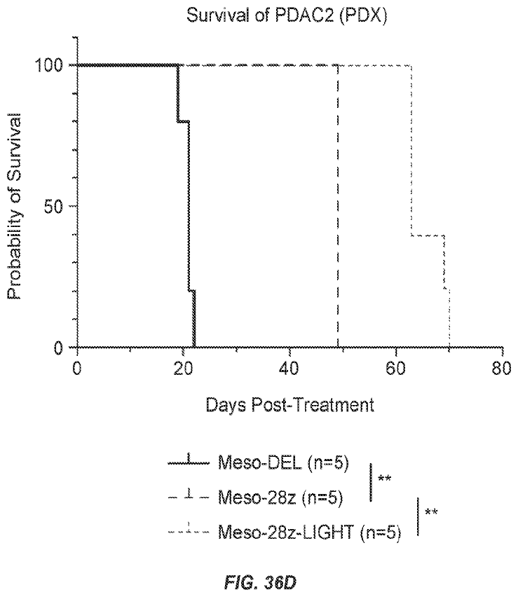

The PDAC2 PDX, was then transduced with a luciferase transgene to allow for in vivo tracking of tumor burden by bioluminescence. Tumor was engrafted orthotopically (intra-pancreatic), and after 7 days for tumor engraftment, tumor-bearing mice were treated with CAR T cells (FIG. 36A). As previously observed, non-functional Meso-DEL CAR T cells lacked therapeutic efficacy, while LIGHT-CAR T cells induced a delayed progression of tumor outgrowth as compared with second-generation CAR T cells (FIGS. 36B and 36C). Complete tumor eradication was not observed, perhaps owing to the low CAR T cell dose used in this model compared with the other preclinical mouse models. However, size reduction or delayed progression of the tumor correlated with a significant survival benefit for mice treated with LIGHT-CAR T cells (FIG. 36D).

### LIGHT-CAR T Cells Display No Adverse Effect in Immunocompetent Mouse Models and Non-Cancerous Cell Types

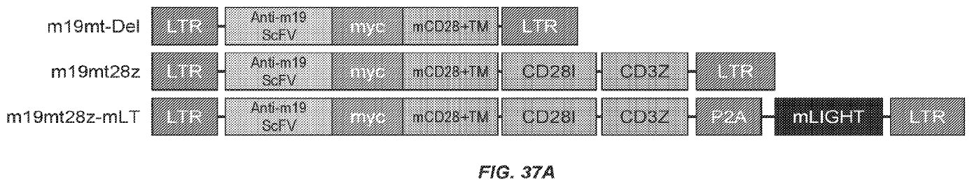

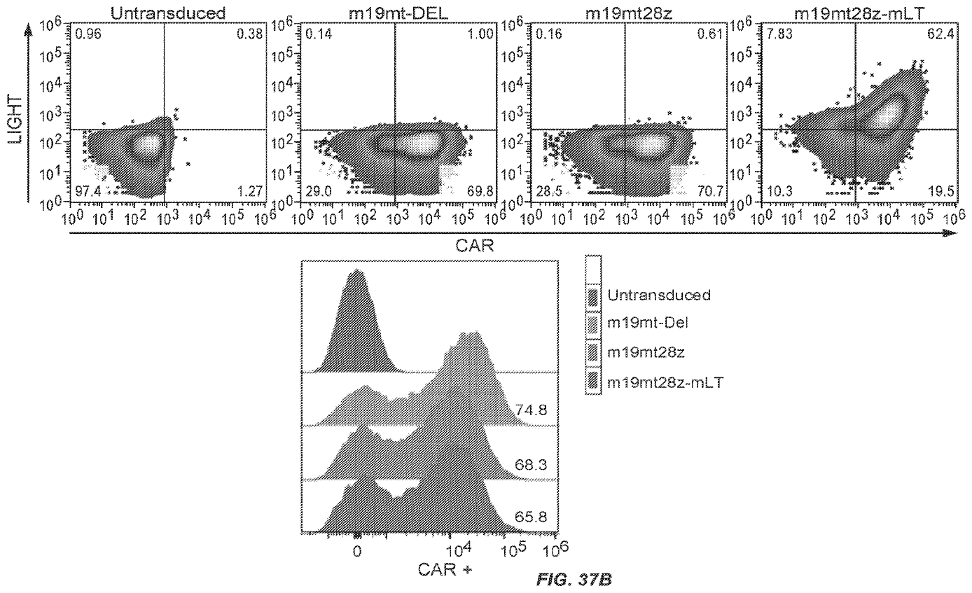

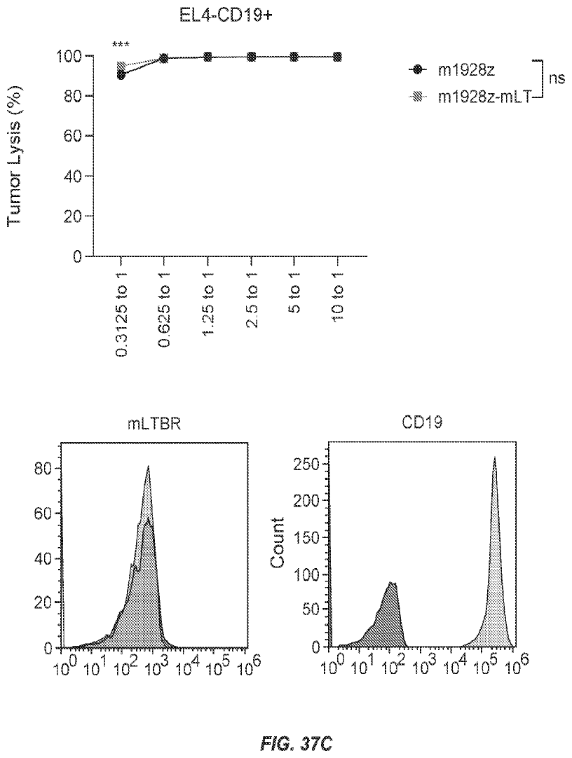

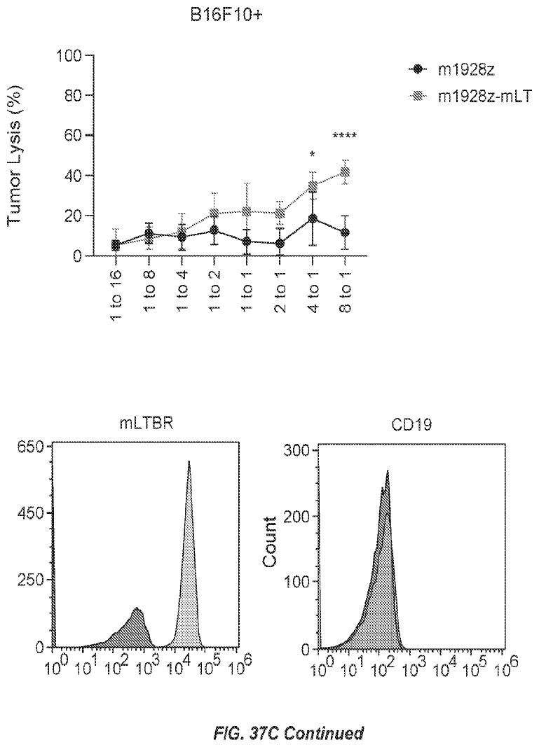

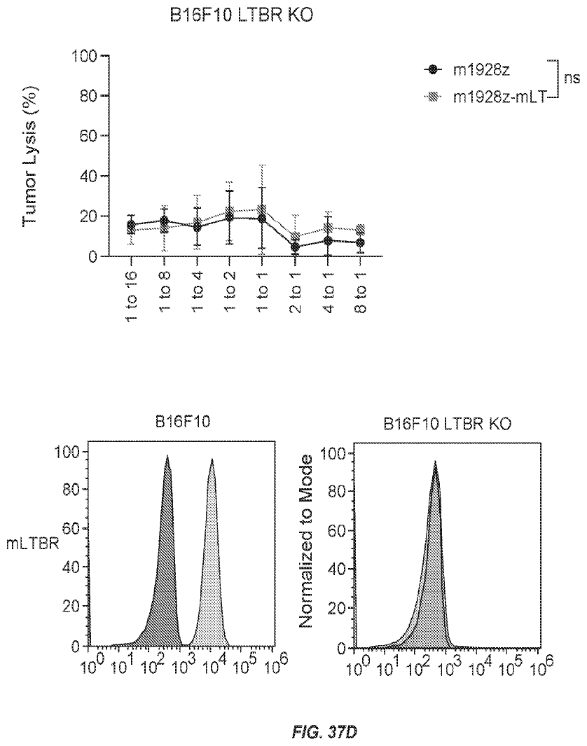

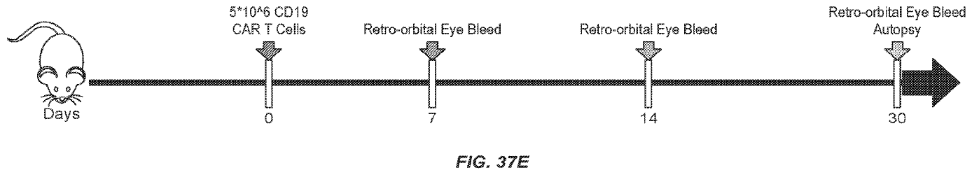

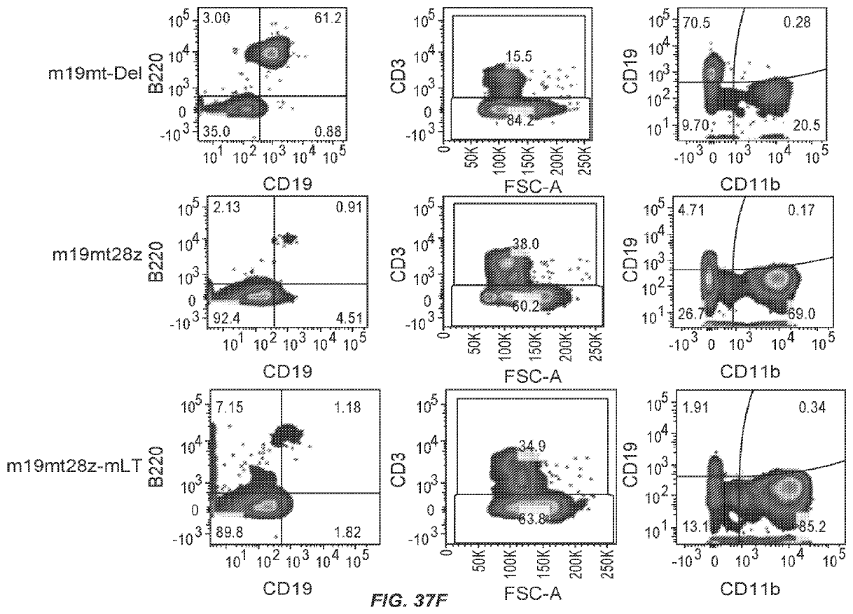

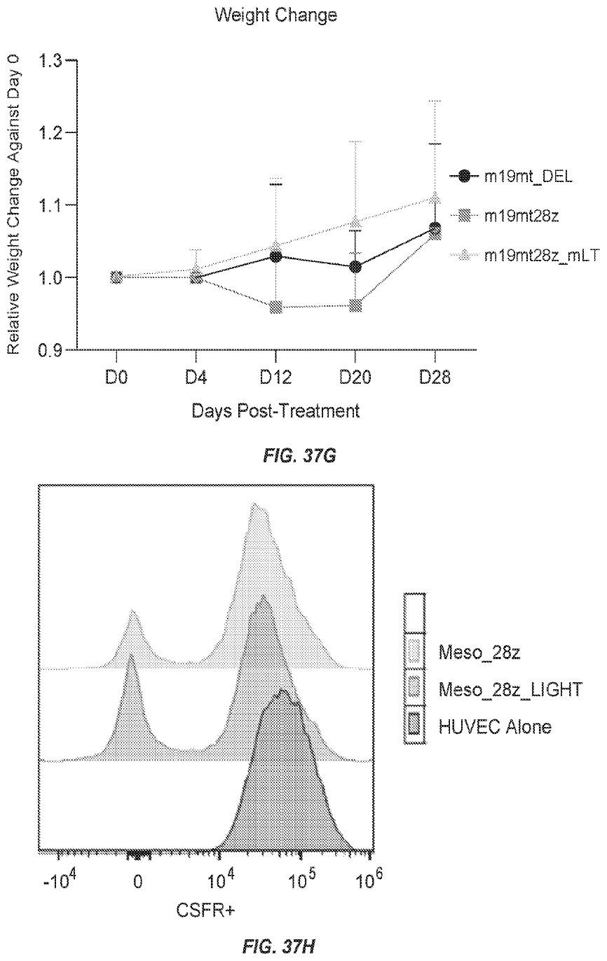

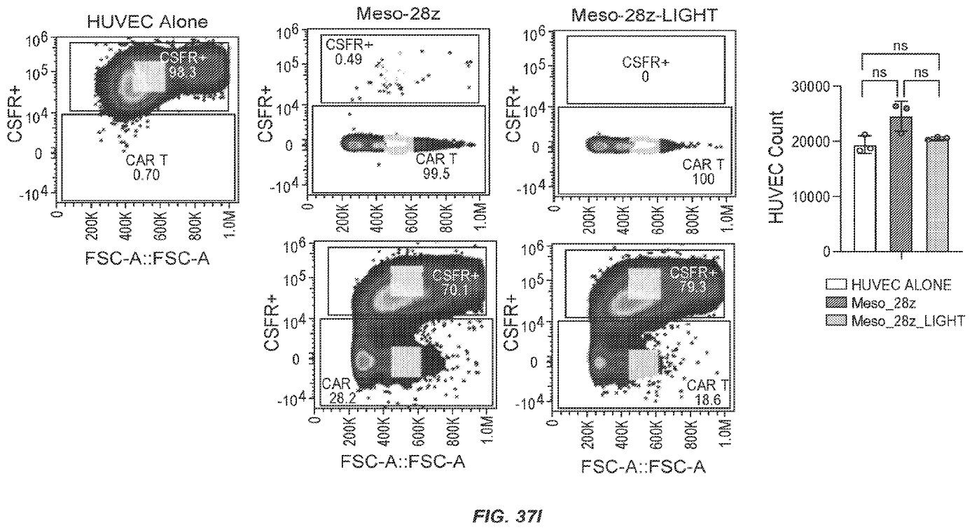

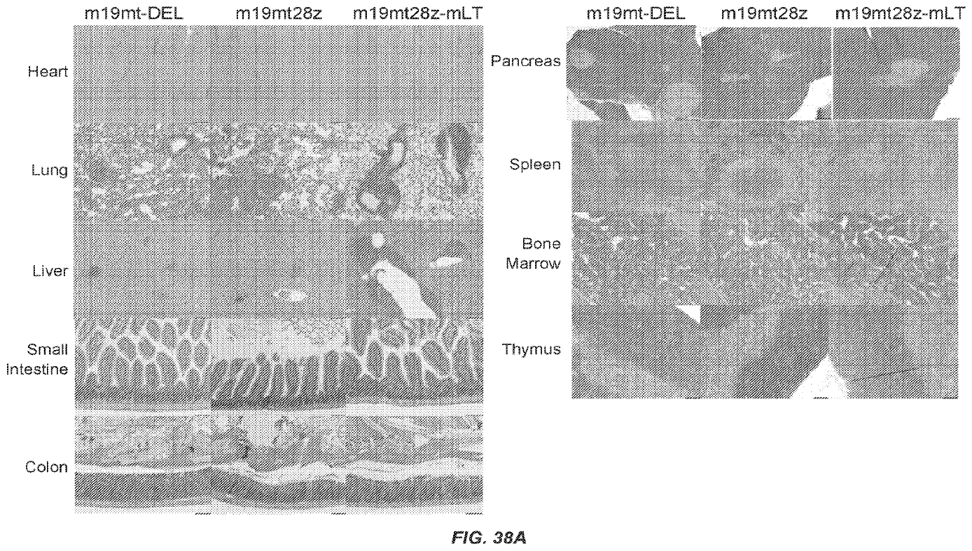

Given that LIGHT can elicit cytotoxicity in an LTβR-dependent manner, and LTβR expression was not restricted to tumor cells, adverse effects were evaluated, including any overt toxicities or histological abnormalities, of LIGHT-CAR T cells in vivo. A fully immunocompetent mouse model was used and mCAR T cell constructs directed against mCD19 were used as these mCAR constructs had been well characterized and validated in the development of other armored CAR T cell platforms. Moreover, B-cell aplasia derived from CAR T cell activity could be used as a proxy for in vivo CAR T cell activity in the absence of tumor. Mouse CD19 CAR T cell constructs with and without mouse LIGHT (mLIGHT) were designed with myc tag to allow for detection of CAR T cells (FIG. 37A). m19mt-DEL, lacked the intracellular signaling portion of the CAR and served as a negative control. Mouse T cells were successfully transduced with these constructs and both CAR and mLIGHT expression were validated (FIG. 37B).

To evaluate the cytotoxicity of our mouse mCD19 LIGHT-CAR T cells and enhanced cytolytic capacity conferred by mLIGHT, the following cell lines were used: EL4, a mouse thymoma line that overexpresses CD19 and B16F10, a mouse melanoma cell line. B16F10 melanoma did not express CD19, but expressed mLTβR, the target receptor for mLIGHT. As expected, complete elimination of the CD19⁺ EL4 cell line was observed by bioluminescence and better tumor killing of mouse melanoma B16F10 was observed using LIGHT-CAR T cells compared with second-generation CAR T cells (FIG. 37C). Further validating the effect of LIGHT, when mLTBR was knocked-out from B16F10, the enhanced cytolysis of LIGHT-CAR T cells was abolished (FIG. 37D).

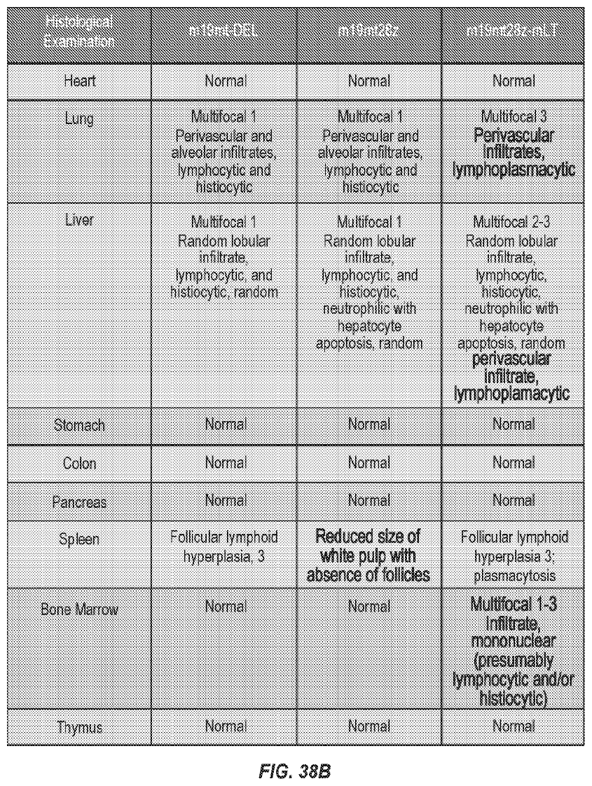

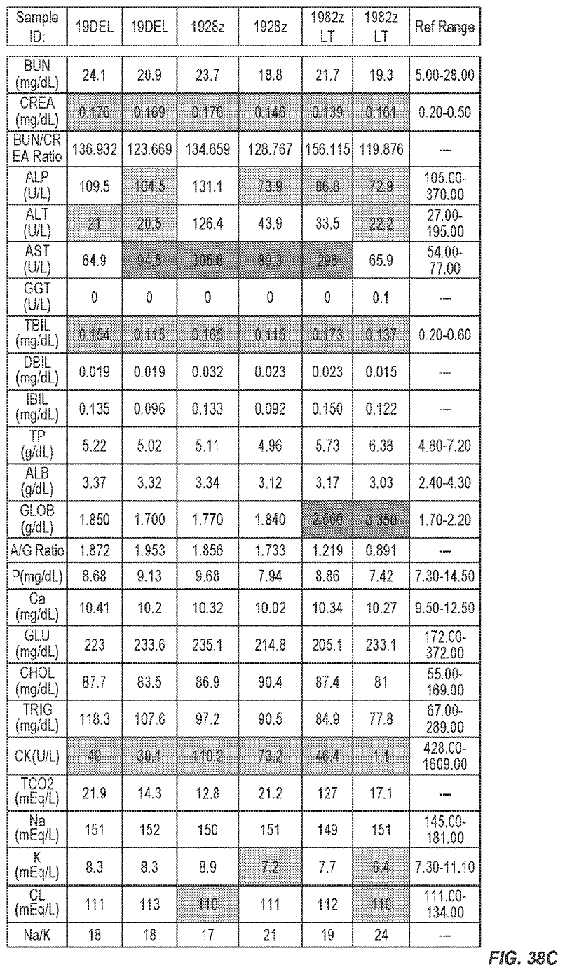

Mouse CD19-directed CAR T cells were injected into C57/BL6 mice and peripheral blood was collected at 7, 14, and 30 days following CAR T cell injection. Full necropsy assessment after day 30, for any abnormal histopathology or serum chemistry, demonstrated no significant toxicity associated with LIGHT-CART cells (FIG. 37E and FIG. 38A). Flow cytometry analysis of the peripheral blood demonstrated B-cell aplasia following CD19-directed CAR T cells with and without LIGHT resulted in B cell aplasia, but none associated with m19mt-DEL with non-functional CAR construct (FIG. 37F). By day 30, no toxicity (significant weight loss, paralysis, neurotoxicity, and GvHD symptoms) was observed associated with LIGHT-CAR T cells (FIG. 37G). Lymphocytic infiltration increased slightly in some tissues, and higher globulin level was associated with higher inflammation state, suggesting biological effects from LIGHT in the immunocompetent mouse model (FIGS. 38B and 38C). To evaluate whether the LIGHT-CAR T cells were cytotoxic to normal cells, human umbilical vein endothelial cells (HUVEC) were stained, selected as a normal cell with LTβR expression, with CellTrace Far Red (CSFR) dye prior to co-culture with CAR T cells. After 72 hours, the cells were collected for flow cytometry analysis, and the amount of CSFR+ cells was quantified (FIG. 37H). There was no significant decrease in HUVEC numbers after co-culture with LIGHT-CAR T cells (FIG. 37I). Collectively, these data indicated that LIGHT-CAR T cells displayed no adverse effects in immunocompetent mouse models and non-cancerous cell types.

### Discussion

Although CAR T cells have shown great success in the treatment of B-cell malignancies, response rates in patients with solid tumors have been underwhelming. The reasons for this include the lack of suitable target antigens, tumor heterogeneity, poor CAR T cell trafficking, poor infiltration of tumors, and an immunosuppressive tumor microenvironment. Importantly, the heterogeneous antigen expression of solid tumors renders antigen-specific CAR T cells ineffective, leading to antigen escape and antigen-negative disease relapse (Majzner et al., Cancer Discov. (2018); 8 (10): 1219-26, 21; Junttila et al., Nature. (2013); 501 (7467): 346-54). Novel strategies to overcome antigen-heterogeneity and antigen escape have the potential to improve responses to CAR T cell therapies in both solid tumor and hematologic malignancies.

For the past two decades, investigators have explored method to deliver LIGHT as an antineoplastic therapy, including through bacterial and viral vectors, as well as fusion proteins (Yu et al., J Immunol. (2007); 179 (3): 1960-8; Hu et al., Cell Mol Immunol. (2010); 7 (4): 296-305; Yan et al., Prostate. (2015); 75 (3): 280-91; Johansson-Percival et al., Nat Immunol. (2017); 18 (11): 1207-17; Tang et al., Cancer Cell. (2016); 29 (3): 285-96). Tumor volume reduction and primary tumor elimination have been observed in multiple preclinical models, but LIGHT induced, direct tumor-specific killing has not been characterized. Here, we describe the overexpression and delivery of LIGHT via CAR T cell therapy. Prior work has demonstrated that overexpression of LIGHT in transgenic mice results in a hyper-activated T cell state, increasing the risk of spontaneous autoimmunity (Wang et al., J Clin Invest. (2001); 108 (12): 1771-80). However, CAR T cells can act as “micropharmacy” tumor-directed delivery systems to minimize systemic toxicity, while providing biologically active molecules to the tumor site (Rafiq et al., Nat Biotechnol. (2018); 36 (9): 847-56; Gardner et al., Nat Chem Biol. (2022); 18 (2): 216-25; Jaspers et al., J Clin Invest. (2023); 133 (9)). In addition to LIGHT-mediated tumor cytotoxicity, LIGHT-CAR T cells have improved activation, proliferation, and persistence compared with second-generation CAR T cells.

In this study, LIGHT-CAR T cells eradicated antigen-heterogeneous cancer cells via a unique orthogonal method of tumor cell lysis. The inventors first targeted the AML-associated antigen, CD371 (CLEC12A) on AML blasts and leukemic stem cells, which present with a relatively heterogeneous antigen expression of CD371, versus the relatively uniform expression of CD19 by B-ALL and of BCMA by MM (Boyd et al., Cancer Cell. (2018); 34 (3): 483-98 e5). The CD371-directed LIGHT-CAR T cells exhibited enhanced cytotoxicity compared with second-generation CD371-directed CAR T cells. Moreover, augmented cytolysis occurred in both antigen low and antigen knockout settings, demonstrating that LIGHT-CAR T cells can prevent the outgrowth of antigen-negative tumor cells in vitro. Similar augmented cytotoxicity of LIGHT-CAR T cells was observed in a variety of solid tumor cell lines, where they eliminated antigen-heterogeneous populations of cancer cells and conferred superior tumor control and a concomitant survival benefit in xenograft models of PDAC.

Additionally, it was found that the LIGHT/LTβR signaling axis is important for the cytotoxicity of LIGHT-CAR T cells. Systematic deletion of known receptors for LIGHT from tumor cells abolished the augmented cytotoxicity of LIGHT-CAR T cells in LTßR knockout cell lines. Overexpression of a truncated, non-signaling version of LTßR in the knockout cells also abolished augmented cytolysis, further validating this finding. Furthermore, the inventors demonstrated increased activation and proliferation profiles of LIGHT-CAR T cells compared with second-generation CAR T cells, and corroborated these results through single-cell multiomics data. These findings collectively suggest that LIGHT-CAR T cells have greater antitumor potency, expansion, and persistence in the context of antigen-heterogeneous malignancies.

A recent study employing genome-wide screening to identify synthetic drivers of T cell activation and proliferation demonstrated increased proliferation and effector functions associated with overexpression of LTßR (Legut et al., Nature. (2022); 603 (7902): 728-35). This finding suggests that the signaling effects of LIGHT/LTßR axis are cell-type dependent and supports its use to augment T cell function. Additionally, LIGHT's co-stimulatory effect may be pleiotropic, acting on both the CAR T cells and other endogenous immune cells to enhance endogenous antitumor responses (Loeffler et al., Proc Natl Acad Sci USA. (2007); 104 (31): 12879-83). Prior work has demonstrated that LIGHT plays additional roles in the normalization of tumor vasculature and maintenance of both tertiary lymphoid structures and high endothelial venules at tumor sites; these features could drastically enhance the effects of immune effector cell trafficking to and infiltration of tumor mass (Lu et al., Front Immunol. (2014); 5:47; Johansson-Percival et al., Cell Rep. (2015); 13 (12): 2687-98) and are positive indicators of dense lymphocytic infiltrates associated with improved antitumor responses and patient survival (Ramachandran et al., Cancer Cell. (2023); 41 (6): 1134-51 e10). Together, these studies indicate that LIGHT may not only boost the effect of CAR T cell therapy but also enhance existing endogenous antitumor responses by engaging endogenous immune cells, normalizing tumor vasculature, and facilitating the formation of tertiary lymphoid structures and high endothelial venules.

In summary, the inventors developed CAR T cells with LIGHT overexpression capable of robust killing of antigen-heterogeneous tumor cells. These LIGHT-CAR T cells are more activated and proliferative, and secrete more proinflammatory cytokines upon antigen encounter compared with CAR T cells targeting only one specific tumor antigen. These LIGHT-derived enhancements correlate with improved tumor control and survival in multiple in vivo models compared to non-LIGHT CAR T cells. Therefore, we plan to next use syngeneic models to determine how LIGHT-CART Cells interact with a fully immunocompetent model and may augment endogenous immune responses. This approach represents a novel therapeutic strategy to improve the effectiveness of CAR T cells targeting solid tumors, and its translation to the clinic holds promise for improving the outcomes of these patients.

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An immunoresponsive cell comprising:
(a) an antigen-recognizing receptor that binds to an antigen, and
(b) an exogenous LIGHT (homologous to Lymphotoxin, exhibits Inducible expression and competes with HSV Glycoprotein D for binding to Herpesvirus entry mediator, a receptor expressed on T lymphocytes) polypeptide or a fragment thereof.

2. The immunoresponsive cell of claim 1, wherein the exogenous LIGHT polypeptide is constitutively expressed.

3. The immunoresponsive cell of claim 1 or 2, wherein the exogenous LIGHT polypeptide is secreted.

4. The immunoresponsive cell of claim 1 or 2, wherein the exogenous LIGHT polypeptide is not secreted.

5. The immunoresponsive cell of any one of claims 1-4, wherein the antigen recognizing receptor is recombinantly expressed.

6. The immunoresponsive cell of any one of claim 1-5, wherein the antigen-recognizing receptor is expressed from a vector.

7. The immunoresponsive cell of any one of claims 1-6, wherein the exogenous LIGHT polypeptide is expressed from a vector.

8. The immunoresponsive cell of any one of claims 1-7, wherein the cell is selected from the group consisting of a T cell, a Natural Killer (NK) cell, and a stem cell from which a lymphoid cell may be differentiated.

9. The immunoresponsive cell of claim 8, wherein the cell is a T cell.

10. The immunoresponsive cell of claim 8 or 9, wherein the T cell is selected from the group consisting of a cytotoxic T lymphocyte (CTL), a regulatory T cell, and a Natural Killer T (NKT) cell.

11. The immunoresponsive cell of claim 8, wherein the stem cell is a pluripotent stem cell.

12. The immunoresponsive cell of any one of claims 1-11, wherein the immunoresponsive cell is autologous.

13. The immunoresponsive cell of any one of claims 1-12, wherein the immunoresponsive cell is allogeneic.

14. The immunoresponsive cell of any one of claims 1-13, wherein the antigen is a tumor antigen or a pathogen antigen.

15. The immunoresponsive cell of claim 14, wherein the antigen is a tumor antigen.

16. The immunoresponsive cell of claim 14 or 15, wherein the tumor expresses LTβR or a portion thereof.

17. The immunoresponsive cell of claim 16, wherein the tumor is selected from the group consisting of acute myeloid leukemia (AML), pancreatic cancer, ovarian cancer, lung cancer, melanoma, colon cancer, mesothelioma, osteosarcoma, cholangiocarcinoma, colorectal cancer, and breast cancer.

18. The immunoresponsive cell of claim 14 or 15, wherein the tumor is blood cancer.

19. The immunoresponsive cell of claim 18, wherein the blood cancer is selected from the group consisting of acute myeloid leukemia (AML), chronic lymphocytic leukemia, B cell leukemia, multiple myeloma, lymphoblastic leukemia (ALL), Hodgkin's lymphoma, and non-Hodgkin's lymphoma.

20. The immunoresponsive cell of claim 14 or 15, wherein the tumor is a solid tumor.

21. The immunoresponsive cell of claim 20, wherein the solid tumor is selected from the group consisting of pancreatic cancer, ovarian cancer, lung cancer, melanoma, colon cancer, colorectal cancer, mesothelioma, osteosarcoma, cholangiocarcinoma, and breast cancer.

22. The immunoresponsive cell of claim 14 or 15, wherein the tumor antigen is selected from the group consisting of CD371, mesothelin, CD19, MUC16, MUC1, CAIX, CEA, CD8, CD7, CD10, CD20, CD22, CD30, CLL1, CD33, CD34, CD38, CD41, CD44, CD49f, CD56, CD74, CD123, CD133, CD138, EGP-2, EGP-40, EpCAM, Erb-B2, Erb-B3, Erb-B4, FBP, Fetal acetylcholine receptor, folate receptor-α, GD2, GD3, HER-2, hTERT, IL-13R-α2, κ-light chain, KDR, LeY, L1 cell adhesion molecule, MAGE-A1, MAGEA3, p53, MARTI, GP100, Proteinase3 (PR1), Tyrosinase, Survivin, EphA2, NKG2D ligands, NY-ESO-1, oncofetal antigen (h5T4), PSCA, PSMA, ROR1, TAG-72, VEGFR2, WT-1, BCMA, NKCS1, EGF1R, and EGFR-VIII.

23. The immunoresponsive cell of claim 22, wherein the tumor antigen is CD371.

24. The immunoresponsive cell of claim 22, wherein the tumor antigen is mesothelin.

25. The immunoresponsive cell of any one of claims 1-24, wherein the antigen-recognizing receptor is a T cell receptor (TCR) or a chimeric antigen receptor (CAR).

26. The immunoresponsive cell of claim 25, wherein the antigen-recognizing receptor is a chimeric antigen receptor (CAR).

27. The immunoresponsive cell of claim 25 or 26, wherein the CAR comprises an extracellular antigen-binding domain, a transmembrane domain, and an intracellular domain.

28. The immunoresponsive cell of claim 27, wherein the extracellular antigen-binding domain comprises an antigen-binding fragment that binds to the antigen.

29. The immunoresponsive cell of claim 28, wherein the antigen-binding fragment is a Fab, (Fab)₂, variable fragment (Fv), or a single chain variable fragment (scFv).

30. The immunoresponsive cell of claim 27, wherein the transmembrane domain comprises a CD8 polypeptide, a CD28 polypeptide, a CD32 polypeptide, a CD4 polypeptide, a 4-1BB polypeptide, an OX40 polypeptide, an ICOS polypeptide, or a combination thereof.

31. The immunoresponsive cell of claim 27, wherein the transmembrane domain comprises a CD28 polypeptide.

32. The immunoresponsive cell of claim 27, wherein the intracellular domain comprises a CD3ζ polypeptide.

33. The immunoresponsive cell of claim 27, wherein the intracellular domain further comprises at least one co-stimulatory signaling region.

34. The immunoresponsive cell of claim 33, wherein the at least one co-stimulatory signaling region comprises a CD28 polypeptide, a 4-1BB polypeptide, an OX40 polypeptide, an ICOS polypeptide, a DAP-10 polypeptide, or a combination thereof.

35. The immunoresponsive cell of claim 34, wherein the at least one co-stimulatory signaling region comprises a CD28 polypeptide.

36. The immunoresponsive cell of any one of claims 1-35, wherein the LIGHT polypeptide comprises an amino acid sequence that is at least about 80%, at least about 85%, at least about 90%, at least about 95%, at least about 99%, or at least about 100% identical to the amino acid sequence set forth in SEQ ID NO: 7, SEQ ID NO: 8, or SEQ ID NO: 9.

37. The immunoresponsive cell of any one of claims 1-36, wherein the LIGHT polypeptide comprises the amino acid sequence set forth in SEQ ID NO: 7, SEQ ID NO: 8, or SEQ ID NO: 9.

38. The immunoresponsive cell of any one of claims 1-37, wherein the exogenous LIGHT polypeptide enhances an immune response of the immunoresponsive cell.

39. The immunoresponsive cell of any one of claims 1-38, wherein the exogenous LIGHT polypeptide increases anti-tumor cytokine production of the immunoresponsive cell.

40. The immunoresponsive cell of claim 39, wherein the anti-tumor cytokine is selected from the group consisting of IL-33, IL-5, IL-9, IL-13, IL-2, granulocyte macrophage colony-stimulating factor (GM-CSP), IFN-γ, TNF-super family, Fas ligand (Fas-L), TNF-α, perforin, granzyme B, and granzyme A.

41. A composition comprising the immunoresponsive cell of any one of claims 1-40.

42. The composition of claim 41, which is a pharmaceutical composition comprising a pharmaceutically acceptable excipient.

43. A method of inhibiting growth of a cell that expresses lymphotoxin-β receptor (LTβR) or a portion thereof, the method comprising contacting a cell that expresses LTβR or a portion thereof with the immunoresponsive cell of any one of claims 1-40 or the composition of claim 41 or 42.

44. The method of claim 43, wherein the cell is a tumor cell.

45. A method of reducing tumor burden in a subject, the method comprising administering to a subject suffering from a tumor the immunoresponsive cell of any one of claims 1-40 or the composition of claim 41 or 42.

46. The method of claim 45, wherein the method reduces the number of tumor cells, reduces tumor size, and/or eradicates the tumor in the subject.

47. A method of treating and/or preventing a tumor, the method comprising administering to a subject suffering from a tumor the immunoresponsive cell of any one of claims 1-40 or the composition of claim 41 or 42.

48. A method of lengthening survival of a subject having a tumor, the method comprising administering to the subject the immunoresponsive cell of any one of claims 1-40 or the composition of claim 41 or 42.

49. The method of any one of claims 44-48, wherein the tumor expresses LTβR or a portion thereof.

50. The method of claim 39, wherein the tumor is selected from the group consisting of pancreatic cancer, acute myeloid leukemia (AML), ovarian cancer, lung cancer, melanoma, colon cancer, colorectal cancer, mesothelioma, osteosarcoma, cholangiocarcinoma, and breast cancer.

51. The method of any one of claims 44-50, wherein the tumor is a cancer.

52. The method of any one of claims 44-48 and 51, wherein the tumor is a blood cancer.

53. The method of claim 52, wherein the blood cancer is selected from the group consisting of acute myeloid leukemia (AML), chronic lymphocytic leukemia, B cell leukemia, multiple myeloma, lymphoblastic leukemia (ALL), and non-Hodgkin's lymphoma.

54. The method of any one of claims 44-48 and 51, wherein the tumor is a solid tumor.

55. The method of claim 54, wherein the solid tumor is selected from the group consisting of pancreatic cancer, acute myeloid leukemia (AML), ovarian cancer, lung cancer, melanoma, colon cancer, colorectal cancer, mesothelioma, osteosarcoma, cholangiocarcinoma, and breast cancer.

56. The method of any one of claims 44-55, wherein the tumor expresses low detectable level of the antigen to which the antigen recognizing receptor binds.

57. The method of claim 56, wherein the low detectable level is less than about 1500 molecules per cell.

58. The method of claim 56, wherein the low detectable level is less than about 1000 molecules per cell.

59. A method of increasing immune-activating cytokine production in response to an antigen in a subject, the method comprising administering to a subject in need thereof the immunoresponsive cell of any one of claims 1-40 or the composition of claim 41 or 42.

60. The method of claim 59, wherein the immune-activating cytokine is selected from the group consisting of IL-33, IL-5, IL-9, IL-13, IL-2, granulocyte macrophage colony-stimulating factor (GM-CSF), IFN-γ, TNF-super family, OX40, Fas ligand (Fas-L), TNF-α, perforin, granzyme B, and granzyme A.

61. A method of treating a pathogen infection or an infectious disease in a subject, the method comprising administering to a subject suffering from a pathogen infection or an infectious disease the immunoresponsive cell of any one of claims 1-40 or the composition of claim 41 or 42.

62. The method of any one of claims 45-61, wherein the subject is a human subject.

63. A nucleic acid composition comprising (a) a first nucleic acid encoding an antigen-recognizing receptor and (b) a second nucleic acid encoding an exogenous LIGHT polypeptide or a fragment thereof.

64. The nucleic acid composition of claim 63, wherein the first nucleic acid is operably linked to a first promoter.

65. The nucleic acid composition of claim 63 or 64, wherein the second nucleic acid is operably linked to a second promoter.

66. The nucleic acid composition of 64 or 65, wherein the first promoter is the same as the second promoter.

67. The nucleic acid composition of any one of claims 64-66, wherein the first promoter and/or the second promoter are a constitutive promoter.

68. The nucleic acid composition of claim 67, wherein the constitutive promoter is selected from the group consisting of a retroviral LTR, a CMV promoter, an EF1α promoter, a SV40 promoter, a PGK1 promoter, a Ubc promoter, a β-actin promoter, and a CAG promoter.

69. A vector comprising the nucleic acid composition of any one of claims 63-68.

70. The vector of claim 69, wherein the vector is a retroviral vector.

71. A cell comprising the nucleic acid composition of any one of claims 63-68.

72. A cell comprising the vector of claim 69 or 70.

73. A method for producing an antigen-specific immunoresponsive cell, the method comprising introducing into an immunoresponsive cell the nucleic acid composition of any one of claims 63-68 or the vector of claim 69 or 70.

74. A method for producing an antigen-specific immunoresponsive cell, the method comprising introducing into an immunoresponsive cell comprising antigen-recognizing receptor a nucleic acid encoding an exogenous LIGHT polypeptide or a fragment thereof.

75. A kit comprising an immunoresponsive cell of any one of claims 1-40 or the composition of claim 41 or 42, the nucleic acid composition of any one of claims 63-68, or the vector of claim 69 or 70.

76. The kit of claim 75, wherein the kit further comprises written instructions for treating a tumor a pathogen infection, and/or an infectious disease.

77. The immunoresponsive cell of any one of claims 1-40 for use in a therapy.

78. The immunoresponsive cell of any one of claims 1-40 for use in inhibiting growth of a cell expressing LTβR or a portion thereof, reducing tumor burden in a subject, treating a tumor in a subject, lengthening survival of a subject having a tumor, and/or increasing immune-activating cytokine production in response to an antigen in a subject.

79. The composition of claim 41 or 42 for use in a therapy.

80. The composition of claim 41 or 42 for use in inhibiting growth a cell expressing LTβR or a portion thereof, reducing tumor burden in a subject, treating a tumor in a subject, lengthening survival of a subject having a tumor, and/or increasing immune-activating cytokine production in response to an antigen in a subject.

81. Use of the immunoresponsive cell of any one of claims 1-40 in the manufacture of a medicament for inhibiting growth a cell expressing LTβR or a portion thereof, reducing tumor burden in a subject, treating a tumor in a subject, lengthening survival of a subject having a tumor, and/or increasing immune-activating cytokine production in response to an antigen in a subject.

82. Use of the composition of claim 41 or 42 in the manufacture of a medicament for inhibiting growth a cell expressing LTβR or a portion thereof, reducing tumor burden in a subject, treating a tumor in a subject, lengthening survival of a subject having a tumor, and/or increasing immune-activating cytokine production in response to an antigen in a subject.
